(19) United States
(12) Patent Application Publication
(10) Pub. No.: US 2025/0136973 A1
(43) Pub. Date: May 1, 2025

(54) SINGLE-CELL LINEAGE TRACKING, TEMPORAL RECORDING, AND CRISPR SCREENING PLATFORM

(71) Applicant: VANDERBILT UNIVERSITY, Nashville, TN (US)

(72) Inventors: Md Mirazul ISLAM, Nashville, TN (US); Ken S. LAU, Nashville, TN (US); Robert J. Coffey, JR., Nashville, TN (US)

(21) Appl. No.: 18/929,057

(22) Filed: Oct. 28, 2024

Related U.S. Application Data
(60) Provisional application No. 63/593,342, filed on Oct. 26, 2023.

(51) Int. Cl. C12N 15/10

(52) U.S. Cl. CPC C12N 15/1082; C12N 15/1065; C12N 15/1089

(57) ABSTRACT

Disclosed herein is a custom platform called NSC-seq that can be used for cell lineage and cell division tracking in a temporal fashion but also with single-cell resolution. With simultaneous integration of single-cell transcriptomics with lineage barcoding and temporal recording information, NSC-seq facilitates the generation of multidimensional datasets for elucidating functional heterogeneity and clonal events in vivo. We apply this platform (i) to decipher lineage branching of mouse embryonic development, (ii) to record clonal dynamics of the adult intestinal epithelium, and (iii) to track clonal composition of the mouse intestinal tumors. In addition, we apply NSC-seq to assess a comprehensive gene expression phenotype for individual gene knockout at the single-cell level using existing whole-genome CRISPR knockout screening plasmid libraries. Overall, NSC-seq enables in vivo temporal recording of mammalian development and cancer at single-cell resolution.

### CROSS-REFERENCE TO RELATED APPLICATIONS

This application claims benefit of U.S. Provisional Application No. 63/593,342, filed Oct. 26, 2023, which is hereby incorporated herein by reference in its entirety.

### STATEMENT OF GOVERNMENT INTEREST

This invention was made with Government Support under Grant Nos. DK103831 and CA236733 awarded by the National Institutes of Health. The Government has certain rights in the invention.

### SEQUENCE LISTING

This application contains a sequence listing filed in ST.26 format entitled “222230-1420 Sequence Listing” created on Oct. 21, 2024, and having 17,612 bytes. The content of the sequence listing is incorporated herein in its entirety.

### BACKGROUND OF THE INVENTION

CRISPR-Cas9-based single-cell technologies have been applied to address a wide range of biological questions, including molecular recording of embryonic development, genetic screening, and tracking tumor evolution at the single-cell resolution. Molecular events can be recorded into DNA as genetic edits to the genome using Cas9. Cellular events can be dynamic and stochastic in nature, and thus, developing a molecular clock system is very useful to order and quantify these events.

### SUMMARY OF THE INVENTION

Disclosed herein is a custom platform called NSC-seq that can be used for cell lineage and cell division tracking in a temporal fashion but also with single-cell resolution. With simultaneous integration of single-cell transcriptomics with lineage barcoding and temporal recording information, NSC-seq facilitates the generation of multidimensional datasets for elucidating functional heterogeneity and clonal events in vivo. We apply this platform (i) to decipher lineage branching of mouse embryonic development, (ii) to record clonal dynamics of the adult intestinal epithelium, and (iii) to track clonal composition of the mouse intestinal tumors. In addition, we apply NSC-seq to assess a comprehensive gene expression phenotype for individual gene knockout at the single-cell level using existing whole-genome CRISPR knockout screening plasmid libraries. Overall, NSC-seq enables in vivo temporal recording of mammalian development and cancer at single-cell resolution.

Disclosed herein is a gRNA capture primer comprising from 3′ to 5′ a capture polynucleotide complementary to the 3′-end of a gRNA scaffold having the nucleic acid sequence GACTCGGTGCCACTTTTTCAAG (SEQ ID NO:1), a cellular barcode, a unique molecular identifier (UMI), and optionally a T7 promoter sequence.

Also disclosed herein is a native gRNA capture and sequencing (NSC-seq) method for single-cell RNA profiling of pooled gRNA: (a) reverse transcribing the pooled gRNA with a native gRNA capture and sequencing (NSC-seq) primer comprising from 3′ to 5′ a capture polynucleotide complementary to the 3′-end of the gRNA scaffold having the nucleic acid sequence GACTCGGTGCCACTTTTTCAAG (SEQ ID NO:1), a cellular barcode, and a unique molecular identifier (UMI) to produce a cDNA; (b) adding an additional sequence to the 3′ end of the cDNA during reverse transcription using a template switch oligonucleotide for library amplification; (c) amplifying the cDNA by polymerase chain reaction (PCR); and (d) sequencing the cDNA.

Single-cell pooled CRISPR screen approach could also be modified to perform bulk CRISPR screening using same NSC-seq capture sequence. Bulk CRISPR screen using sgRNA readout could be an orthogonal alternative to current widely used bulk CRISPR screen using sgRNA corresponding DNA sequencing.

Also disclosed is a method for single-cell screening of pooled gRNA perturbations comprising: (a) introducing one or more gRNA perturbation constructs encoding for one or more sequence specific perturbations to a plurality of cells in a population of cells, wherein each cell in the plurality of the cells receives at least 1 perturbation and wherein each gRNA perturbation construct encodes for an RNA sequence comprising a sequence identifying the perturbation; (b) detecting endogenous mRNAs for each single cell in the plurality of cells using single-cell RNA-seq; (c) detecting gRNA perturbations for each single cell in the plurality of cells using the NSC-seq method of claim 2; and (d) comparing gRNA perturbations to mRNA for each single cell.

In some embodiments, the gRNA is a self-mutating homing guide RNA (hgRNA).

In some embodiments, the method further involves temporal tracking mutations in the hgRNA over cell divisions in a single organism.

In some embodiments, the method further involves lineage tracking mutations in the hgRNA to examine the relationships between organs and tissue layers in an organism.

In some embodiments, the population of cells comprises ex vivo or in vitro cells.

In some embodiments, each cell is in a microfluidic system; and/or wherein each cell is in a droplet.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIGS. 1A to 1J show optimization of a multi-purpose single-cell capture platform. FIG. 1A shows guide RNA (gRNA) capture schematic for the NSC-seq platform. Target site of gRNA scaffold anneals to NSC-seq capture sequence (CS) with a cellular barcode and unique molecular identifier (UMI). An additional sequence (gray) is added to the 3′-end of the cDNA via template switching during reverse transcription to enable downstream library amplification. This gRNA capture approach is compatible with any type of gRNAs (sgRNA, hgRNA, and stgRNA) that contains the target site sequence in the scaffold. FIG. 1B shows Cas9-induced mutations recovery by direct hgRNA capture as compared to mutations detected in DNA of the same samples. FIG. 1C shows guide RNA capture efficiency by NSC-seq assessed in an experiment where all cells from a drug selected cell line should contain sgRNAs. FIG. 1D shows comparative transcriptome capture efficiency between standard inDrop and NSC-seq experiments. FIG. 1E shows NSC-seq experiments on developmentally barcoded whole embryos where Cas9 is constitutively expressed (top). Accumulative mutations on homing barcode regions increase over time (bottom). FIG. 1F shows average mutation density over embryonic time points. Black dot represents geometric mean for each time point and p-value derived from Student's t-test. FIG. 1G shows somatic mitochondrial variants (mtVars) calling from mitochondrial RNA (mtRNA) (top). Approach to filter informative mtVars for lineage tracking using hgRNA mutations as ‘ground truth’ (bottom). FIG. 1H shows the Number of somatic mtVars per cell over embryonic time points. Black dot represents geometric mean for each time point and p-value derived from Student's t-test. FIG. 1I shows Pearson correlation coefficient heat map of variant proportions combining hgRNAs and mtVars for selected tissue types presented as pseudobulk from an E9.5 embryo. FIG. 1J shows multi-modal application of the NSC-seq platform.

FIGS. 2A to 2D show lineage and temporal recording of mouse embryogenesis. FIG. 2A shows normalized mosaic fraction (MF) of early embryonic mutations (EEMs) heat map for E7.75 embryo to reconstruct lineage relationships within the major germ layers. FIG. 2B shows contribution of different EEMs towards various germ layers at E7.75. FIG. 2C shows clonal contribution from a 1st cell-generation mutation (Clone 1) at E7.75 across individual tissue types (p=1.57⁻¹³, Kolmogorov-Smirnov test for the null hypothesis of symmetry) compared to all other clones aggregated as “Clone 2”. FIG. 2D shows density plots representing cumulative turnover of different tissue types across three embryonic timepoints. The widths of the mutation density distributions represent the variation by which different cell types have proliferated across time points. Ecto, embryonic ectoderm; Meso, embryonic mesoderm; Endo, embryonic endoderm; EMeso, extra-embryonic mesoderm; EEndo, extra-embryonic endoderm; PGC, primordial germ cell.

FIGS. 3A to 3K shows embryonic lineage diversification and gut development. FIG. 3A shows Pearson correlation coefficient heat maps of variant proportions (hgRNAs and mtVars) presented as pseudobulk within hematopoietic and somite cell types from E9.5 (top) and E8.5 (bottom) embryos. FIG. 3B shows multiplex HCR-FISH staining of somite (Twist1) and hematopoietic (Kit) markers in a E9.5 embryo. A cluster of hematopoietic cells (white arrowhead) in somite area is shown in inset image (right). Inset mono-color images represent Dapi (i), Twist1 (ii), and Kit (iii). FIG. 3C shows force-directed layout of foregut cells (E7.75) that are colored by annotated tissue types. HPC, hepatopancreatic cells. Gene expression of HPC (Hnf4a), lung (Foxa2), and thyroid/thymus (Eya1) markers are overlaid here. FIG. 3D shows heat map of differentially expressed genes among three foregut tissue types at E7.75. Tissue type-specific genes are labeled on the right. FIG. 3E shows Pearson correlation coefficient heat map of distinct tissue types from gut region (E7.75) presented as pseudobulk, similar to panel a. FIG. 3F shows force directed layout of midgut and hindgut cells are colored by embryonic time points and regions. FIG. 3G shows visceral endoderm (VE)-intermix score overlay onto FIG. 3F. Quantification of the VE intermix score in hindgut compared to midgut cells (Student's t-test). FIG. 3H shows WNT signaling score overlaid onto FIG. 3F. Correlation analysis between the WNT signaling score (y-axis) and VE-intermix score (x-axis). FIG. 3I shows Pearson correlation coefficient heat maps of gut regions with VE as pseudobulk from E7.75 and E8.5 embryos. FIG. 3J shows distribution of clones across cell types in adult mouse small intestinal epithelium. Number at the top represents the total number of detected clones per cell type. Heat map color represents the number of cells found comprising a clone within a given cell type. A plot (below) showing the fraction of parent and childless clone comprising each cell type. EEC, enteroendocrine cells; CBC, crypt-based columnar cells; eRSC, embryonic revival stem cells; EC; enterocytes; TA, transit-amplifying cells. FIG. 3K contains violin plots of CBC-rooted and eRSC rooted clone sizes.

FIGS. 4A to 4J show clonal origin of human colorectal precancer. FIG. 4A is an overview of experimental design for profiling clonal origin across multiple human datasets. FIG. 4B contains bar plots summarizing the number of APC mutation per polyp using targeted DNA sequencing and whole-exome sequencing (WES). FIG. 4C is an ONCOPRINT plot representing the number of APC mutations across human polyps using WES. Here we only show polyps with at least one deactivating APC mutation. FIG. 3D shows multi-region (punch biopsy) WES of a human CRC sample represents distinct APC mutations and Pearson correlation coefficient heat map (bottom) of somatic mutations within the regions of interest (ROI). FIG. 4E show expected median VAF distribution under different clonal architectures. FIG. 4F shows mosaic X chromosome inactivation patterns in female polyps can delineate the clonal origin of cells using expression-based X-linked somatic clonal SNVs. Male polyps are considered as monoancestrol due to single X chromosome in male. FIG. 4G contains box plots represent distribution of X-linked clonal SNVs (%) between male and female polyps. Red line is a cut-off to assign ancestry in female polyps. FIG. 4H is a summary of median VAF based polyp profiling. FIG. 4I shows Pearson correlation coefficient heat maps of variants (hgRNAs and mtVars) from mouse intestinal tumor (ApcMin/+) derived single cell. Distinctly correlated regions are marked by three clones within the same tumor. (FIG. 4J shows estimated mutation density for the three assigned clones in FIG. 4I. Five tumors (star) show more than 2 distinct Apc mutations.

FIGS. 5A to 5F shows design and validation of NSC-seq platform. FIG. 5A shows a target site (SEQ ID NO:2) for NSC-seq capture sequence (SEQ ID NO:1), along with quality metrics of the capture sequence primer. FIG. 5B shows experimental design of a control lineage tracking experiment using homing CRISPR-barcoded HEK293FT, where the hierarchy of the cultures are known through passage sampling. Similar lineage trees are observed from both bulk DNA and bulk hgRNA barcodes in this experiment (bottom). FIG. 5C is an overview of single-cell experiment using NSC-seq platform simultaneously capturing both gRNA and mRNA within the same droplet. We design custom hydrogel beads for NSC-seq experiment. FIG. 5D shows workflow delineating two separate library preparations (gRNA and mRNA) of NSC-seq. FIG. 5E shows distinct cDNA size selection approaches have different sgRNA capture yield. Two separate library preparation approaches in FIG. 5D result better capture efficiency. FIG. 5F shows comparative transcriptome (mRNA) capture efficiency between inDrop and NSC-seq experiments.

FIGS. 6A to 6H shows overview of temporal recording. FIG. 6A is a schematic representation of increasing mutation density and mutation frequency overtime in self-mutating CRISPR system. Mutation frequency denotes the proportion of wild-type barcodes at a given time. Mutation density is the number of unique mutations per mutated barcode. Color indicates different timepoints. Insertion (capital), deletion (dotted line) and base substitution (underline) mutations are shown here (SEQ ID NOs: 3-17). Theoretical expected mutation frequency and mutation density are function of time (bottom). FIG. 6B is a schematic of in vitro small intestinal (SI) organoids culture over 4 weeks and subsampled to analyze accumulative mutations. FIGS. 6C and 6D show mutation frequency and mutation density show linear increase overtime. FIG. 6E shows mutation density from adult mouse duodenum (SI) shows linear increase overtime (in vivo). FIG. 6F shows comparative mutation density increases in mouse SI between in vivo and in vitro. Value derived from previous linear model (FIG. 6D and FIG. 6E) to plot under same coordinate. Slope (m) indicates relative rate of cell division. In vitro cell division rate in intestinal organoids is almost 3 times higher than the in vivo intestinal epithelial cell division. FIG. 6G shows comparative cell division (mutation density) across different small intestinal epithelial cell types. Here each dot is a technical replicate from same mouse. Transit-amplifying (TA), and enteroendocrine (EEC). These data support the expected notion that enterocyte turnover is higher than Paneth cells. FIG. 6H shows distribution of mutation density per tuft cells reflects only a small fraction of this cell type shows turnover signature.

FIGS. 7A to 7J show mitochondrial variants detection and validation for lineage analysis. FIG. 7A is a schematic of mitochondrial variants (mtVars) based lineage analysis. FIG. 7B is a representative plot of mtVars and hgRNA mutations from same selective group of cells. FIGS. 7C and 7D show pairwise shared hgRNA mutation proportion for each mtVar (top) and density plot of mtVars across dataset (bottom). mtVars distributed in a smaller number of cells (˜1% of dataset) are more informative for lineage inference. FIG. 7E shows mtVars calling from an adult mouse brain (coronal section) special transcriptomics (ST) data. Pearson correlation coefficient heat map of mtVars proportions for distinct tissue layers in mouse left (L) and right (R) brain. Olfactory nerve layer (ONL) in between left, and right is marked as middle (M). Lineage tree suggests that tissue layers are established before L-R axis commitment during brain development. FIG. 7F shows dendrogram of Pearson correlation coefficient heat map using only mtVars (10×ST data) from human breast cancer. mtVars can identify clonal relationship in human breast cancer tissues, as reported copy number based clonal relationship: clone 2 and clone 3 are closely related compared to clone 1. FIG. 7G shows NSC-seq encapsulation of mouse peripheral blood (PB) cells, followed by cell type annotation using marker genes (dot plot). FIG. 7H show Pseudo-bulk lineage tree using only mtVars. FIG. 7I show pseudo-bulk lineage tree using both hgRNA mutations and mtVars. FIG. 7J shows reconstruction of single cell lineage tree using custom LinTiMaT pipeline. Cells are broadly colored by lymphoid and myeloid lineages.

FIG. 8 shows cell type annotation and data quality control metrics for mouse embryos. FIG. 8 contains box plots representing tissue proportions from E7.0, E7.75, and E8.0. Only E7.75 embryo is from this study. Wild-type embryos (E7.0 and E8.0) proportion calculated from GSE122187. FIGS. 8J and 8K contain UMAP plots are colored by unique molecular identifiers (UMIs), number of unique genes detected per cell, mitochondrial gene counts per cell, and predicted doublet score (Scrublet). FIG. 8L shows UMAPs representing cell cycle status per cell.

FIGS. 9A to 9M show temporal recording reveals asymmetric contribution of early embryonic clones to germ layers and tissue types. FIG. 9A is a histogram of the number of cells in which each mutant allele was observed across three embryonic time points (3-ETP). FIG. 9B shows top mutation frequency distribution from a representative barcode (21 bp) of two E9.5 embryos. Mutation code along the x axis is as follows: barcode (BC) number, barcode position (P), mutation type (insertion, I; deletion, D; mismatch, M), mutated base(s). FIG. 9C shows proportion of shared and unique mutations across 3-ETP. FIG. 9D is a scatter plot showing the proportion of unique mutations within each annotated cell types between E7.75 and E8.5 embryos. FIG. 9E shows relatively fast mutation accumulation in small length hgRNAs, as reported before. Data points calculated from two E9.5 embryos. FIG. 9F is a schematic of a phylogenetic tree from early embryo. Mosaic fraction (MF) of somatic early embryonic mutations (EEMs) that found across all three germ layers tracks cell generation (CG) stage. MF represents the fraction of single cells that carry a mutation. FIG. 9G shows distribution of hgRNA mutations that are shared between ≥2 tissue type at E7.75. The earlier a mutation arises during development, more tissue types share that mutation. FIG. 9H shows relationship between MF and CG. FIG. 9I shows EEMs and corresponding approximate CG for E7.75 embryo. Due to possible dropout in single-cell mutation detection, we assigned CG to the next closest CG stage as shown in h. FIG. 9J shows unequal contribution of EEMs towards specific germ layers at E8.5. FIG. 9K shows MF distribution of 10 EEMs (found in >50% of tissue types) showing unequal contributions to specific tissue types at E9.5. The fraction of cells in each tissue contributed by clones C1 to C10 normalized by summing to 100%. FIG. 9L shows simulated data representing symmetric (left) and asymmetric (right) contribution of first two clones to tissue types during embryogenesis. FIG. 9M shows asymmetric contribution of first two clones calculated from E7.75 embryo.

FIGS. 10A to 10C is a catalog of cellular turnover across embryonic time points. FIG. 10A shows comparative mutation density that correspond to cellular turnover between two time points (E7.75 and E8.5). Here we show only a selective list of tissue types. The difference between Primitive blood early vs late at E8.5, imply that this cell type is highly proliferating and/or this cell type is derived from alternative high proliferating progenitors. FIG. 10B shows cellular turnover across cell types at E9.5 embryo. Hematopoietic cell types show relatively high cellular turnover compared to other somatic cell types. FIG. 10C shows consistent increase of gut endoderm cellular turnover across 3-ETP.

FIGS. 11A to 11H show lineage reconstruction of mouse embryogenesis. FIG. 11A shows reconstructed single-cell lineage tree from E7.75 embryo. Leaf cells are colored by germ layer colors and the proportions of cells in the tree are shown as a pie chart (inset). Nodes are colored by dark gray. Each branch represents an independent mutation event. Non-binary tree for all embryos and adult tissues can be found in NSCseq GitHub page. FIG. 11B is a table summarizing the lineage informative mutations (shared between ≥2 cells) detected between two studies that performed similar whole mouse embryonic lineage tracking using constitutive Cas9. Here, we compared only the best reported embryo data between two studies. FIG. 11C shows after combining mtVars with hgRNA mutations, number of cells with lineage informative mutations increases for single-cell lineage tree. Note that there are high variabilities in the proportion of cell that can be used for lineage tree reconstruction among samples due to multiple reasons, including the barcode detection limit, sequencing depth, number of cells captured per experiment, and time required to accumulate mutations. FIG. 11D shows pseudo-bulk lineage analysis for embryonic germ layers. FIG. 11E shows phylogenetic distance proportion was calculated from lineage tree using reported approach. Extraembryonic endoderm (EEndo) shows less distance compared to ectoderm or mesoderm across embryos, supporting nearby proximity to root (zygote). FIG. 11F and distribution of normalized phylogenetic distance (leaf to root) for annotated cell types. Wide distribution of the distance across cell type at E8.5 and E9.5 compared to E7.75, supporting minimal lineage divergence at E7.75 stage. FIG. 11G shows estimated epiblast progenitor number calculated across four embryos. Average epiblast progenitor population is around 28, similar to previous report. High variability may reflect embryo specific constrain in pluripotent cells number that contributes to somatic lineages. FIG. 11H shows proportion of estimated progenitor population between ectoderm and mesoderm. It has been reported that the number of ectoderm progenitor is more than the number of mesoderm progenitor at the epiblast of the prestreak stage mouse embryo.

FIGS. 12A to 12P show somite-derived hematopoiesis. FIG. 12A shows force-directed layout of hematopoietic cell types and somite from E9.5 embryos. FIG. 12B contains dot plots show overexpressing genes in EryPro1 along with yolk sac (Icam2, Kdr, and Gpr182), or endothelial (Pecam1, and Cdh5) genes. EryPro1 doesn't express a recently reported embryonic multipotent progenitor (eMMP) marker Flt3. FIG. 12C is a heat map shows differentially expressed genes among the cell types. Cell type-specific selective list of genes are marked on the right. HSPCs, hematopoietic stem and progenitor cells. FIG. 12D is a volcano plot represents differentially expressed genes (DEGs) between Erythroid and EryPro1 (LCF>2, p value<0.05). Red dots are upregulated in EryPro1, blue dots are upregulated in Erythroid, and black dots are statistically not significant. FIG. 12E shows enriched pathways in EryPro1 group.

FIG. 12F shows cells are marked by EryPro1 score. FIG. 12G shows RNA velocity overlay shows arrow from Somites to EryPro1, supporting cell state transition. FIG. 12H shows MF of EEMs show similar contribution (asterisk) to both somite and EryPro1. FIG. 12I is a heat map represents shared clones (barcode mutations) across three cell types. FIG. 12J shows UMAP co-embedding of blood progenitor cells from E8.5 with E9.5 cells. Arrow shows EryPro1 cluster and arrowhead shows Erythroid cluster. EryPro1 cells from E9.5 are marked by red dotted line (right). EryPro1 population is present in E8.5 embryo. FIG. 12K shows similar as FIG. 12J with blood progenitor cells from E7.75. There is insignificant overlapping population in EryPro1 cluster (arrow), representing EryPro1 is not present yet at E7.75 stage. FIG. 12L shows force-directed layout of blood progenitor cell types with somite at E8.5. EryPro1 assigned from overlapping cluster (arrow) in FIG. 12J. FIG. 12M is a list of gene upregulates in EryPro1 is shown as dot plot. FIG. 12N shows expression of somite and erythroid specific genes are shown here. Somite to EryPro1 transitioning cells show transient expression of both hematopoietic (Gata1) and somite (Twist1) markers. FIG. 12O shows force-directed layout of three cell types and three time points. Cells are colored by Palantir pseudotime trajectory (right).

FIGS. 13A to 13P shows gut endoderm development and progenitor specification. FIG. 12A shows force-directed layout of three endoderm clusters from FIG. 8A. Cells are colored by two embryonic time points. FIG. 13B shows gene expression of definitive endoderm (Sox2, Otx2, and Ccnd2) and visceral endoderm (Afp, Pla2g12b, and Fmr1nb) specific markers. FIGS. 13C to 13D shows based on region specific marker gene expression, DE (dotted line) is divided into three clusters, supporting regionalization of gut endoderm. Here, VE is the combination of embryonic visceral endoderm (emVE), extra-embryonic visceral endoderm (exVE), and yolk sac endoderm (YsE). Heat map of selective gut specific marker genes (y axis) as mean expression for each tissue type (x axis) are shown here (FIG. 13D. FIG. 13E shows force-directed layout of foregut cells from E7.75 embryo. Three clusters are associated with three progenitor population. HPC, hepatopancreatic cells. Gene expression of HPC (Nkx6-1, Afp), lung (Pyy, Sp5), and thyroid/thymus (Foxe1, and Eye2) clusters are shown here. FIG. 13F shows regulon activity is shown across the three tissue types. FIGS. 13G and 13H show force-directed layout of foregut cells from E8.5 embryo. Heat map of selective marker genes (y axis) as mean expression for each tissue type (x axis). FIG. 13I shows force-directed layout of epiblast cells at E7.5. This scRNA-seq data and epiblast annotations are taken from a previous study. Cells are colored by gut progenitor specific markers. FIG. 13J shows force-directed layout of hindgut and midgut cells from three embryonic time points. Cells are colored by three time points and two corresponding tissue types. Midgut (Gata4, Pyy, and Hoxb1) and hindgut (Cdx2, Cdx4, and Hoxc9) specific markers are shown in the bottom. FIG. 13K shows regulon activity of hindgut and midgut cells at E7.75. FIG. 13L shows PALANTIR pseudo-time and CYTOTRACE scores distribution in midgut and hindgut across three time points. FIG. 13M shows normalized Wnt and Bmp signaling gene expression dynamics. X-axis trajectory over pseudo-time shown in FIG. 13L. Points below the plot are the pseudo-time coordinates of cells from each time point colored according to time point as in FIG. 3F. FIG. 13N is a heat map showing differential gene expression between hindgut and midgut at E7.75. Cell type-specific selective list of genes are marked on the right. FIG. 13O is a Venn diagram of genes that were upregulated in both E7.75 and E8.5 time point of hindgut and midgut area. FIG. 13P contains box plots representing normalized expression of Wnt signaling genes between hindgut and midgut for all three time points. Intestinal stem cell marker Lgr5 is overexpressed in hindgut, whereas Lgr4 and Lgr6 are overexpressed in midgut. P-values are derived from Student's t-test.

FIGS. 14A to 14N show lineage convergence during gut endoderm development. FIG. 14A shows force-directed layout of FACS enriched scRNA-seq data with cell type annotation at E8.75 embryos from a previous study. Cells are marked by VE intermix score that we develop from seven reported VE-specific marker genes (right). FIG. 14B shows endoderm cells from E7.75 and E8.5 are marked by VE intermix score. High intermix score in hindgut area supports predominant VE intermix in hindgut. VE marker gene Cthrc1, reported in a previous study, preferentially marks VE intermix cells in hindgut (right). FIG. 14C contains scatter plots representing Wnt signaling gene expression (y-axis) and VE-intermix score (x-axis). Blue line represents fitted linear regression line. FIG. 14D shows discordance in Lgr4 and Lgr5 expression pattern in DE- and VE-derived cells. Here we use data from a previous study. FIG. 14E shows multiplex HCR-FISH co-staining of VE marker gene (Cthrc1) and Wnt target genes (Lgr5) at E9.5 embryo. Inset gut region is adjacent to hindlimb. FIG. 14F shows force-directed layout and re-clustering of two gut endoderm clusters from E9.5 embryos. FIG. 14G shows lineage analysis of gut-derived progenitors. The large intestine (hindgut) and the small intestine (midgut) are in different branch of the dendrogram. FIG. 14H shows an NSC-seq experiment on an E14.5 embryo. UMAP plot of epithelial cells broadly identifies as large intestinal and small intestinal using gene expression. FIG. 14| shows relative proportion of VE-derived cells in large intestine and small intestine clusters are shown here. FIG. 14J is a schematic of barcode-based clonal contribution analysis. If a barcode is present in more than one cell, it's known as parent clone (e.g., Barcode 1 and 2). Whereas, if a barcode present in only one cell, it's known as childless clone (e.g., Barcode 3 and 4). Concept drawn from Bowling et al. The ratio of parent and childless clones is the indicator of relative contribution among the cell types. FIG. 14K shows VE-derived cells show high parent clone ratio, supporting high contribution to epithelial development. Villin+ cells and Smoc2+ cells are used as control. FIG. 14L shows VE-derived cells show relatively high mutation density corresponding to high cellular turnover. FIG. 14M shows developmental lineage analysis of adult mouse gut-derived tissues from two biological replicates using bulk DNA barcodes. Hindgut, midgut, and foregut-derived tissues in dendrogram colors. Hindgut is displayed as a distinct cluster compared to foregut and midgut. FIG. 14N is a schematic of lineage relationship between definitive endoderm (DE) and visceral endoderm (VE). Dotted arrow represents intermix of VE and DE that eventually form gut tube. Adapted from a previous publication.

FIGS. 15A to 15P show clonal dynamics of adult intestinal epithelium. FIG. 15A shows UMAP representation of adult mice small intestinal epithelial cell types. FIG. 15B is a dot plot showing expression of marker genes for annotated cell types. Dot size represents the fraction of cells expressing the gene, and dot color represents normalized mean expression level. FIG. 14C shows cells colored by mouse number. We exclude mouse 2 from barcode analysis due to limited number of hgRNA detection in NSC-seq experiment. FIG. 15D is a list of genes (including Tob2) is used to design eRSC score, which could mark unique epithelial population in UMAP. FIG. 15E shows eRSC score marks enterocyte-related cells (black arrow) in a published study. FIG. 15F shows pseudo-bulk lineage analysis of mouse small intestinal epithelium. FIG. 15G shows MF of EEM from mouse 1 across annotated cell type. FIG. 15H shows single-cell lineage tree of adult intestinal epithelium from mouse 1. Inset table shows the number of estimated progenitors identified from tree topology for major intestinal cell types. FIG. 15l shows UMAP representation of an independent mouse small intestinal epithelium. FIG. 15J is a dot plot showing expression of marker genes for annotated cell types. FIG. 15K is a distribution of cell types across top 22 clones. FIG. 15L is a distribution of clones across cell types. Number at the top represents the total number of detected clones per cell type. Heat map color represents the number of cells found with that clone. Parent and childless clone fraction across major cell type in the bottom. FIG. 15M contains violin plots represent CBC-rooted and eRSC-rooted clone size. FIG. 15N shows the proportion of estimated progenitor population among three cell types in two independent mice. FIG. 15O shows Tob2, one of the eRSC score genes, expression in CBC and eRSC population. FIG. 15P shows wholemount antibody staining of eRSC marker gene Tob2 in mouse small intestinal crypt.

FIGS. 16A to 16S show multi-omic analysis of human colorectal polyps. FIG. 16A shows distribution of human polyps across cohorts. New cohort polyp samples are generated for this study. Old cohorts (DIS and VAL) are reported before and re-analyzed together. FIG. 16B shows the number of APC gene mutation per using targeted DNA sequencing approach. Polyps without any APC mutations are not shown here. FS DEL, frameshift deletion; INS, insertion. FIG. 16C shows quantification of the number of APC mutation (≥3) in two public CRC datasets over tumor progression. FIG. 16D shows two independent approaches show similar somatic mutation detection from scRNA-seq dataset. Here we use transcriptionally assigned abnormal cells (ASC or SSC) to call somatic mutations (SNVs) as pseudo-bulk and polyp infiltrating immune cells's SNVs to remove germline variants from polyps. FIG. 16E is a density plot representing wide distribution of median VAF in polyps using SComatic. FIG. 16F shows VAF distribution of X-linked SNVs in a male (M) polyp. Red line indicates cut-off (0.6) for clonal and subclonal SNVs. FIG. 16G is a simulation experiment, intermixing cells from two or three independent male polyps, showing reduced clonal SNVs (%) depending on the number of polyps intermixed. FIG. 16H contains frequency plots showing proportion of clonal SNVs (%) in two female (F) polyps with known number of APC mutations. FIG. 16I contains scatter plot showing significant correlation between median VAF and X-liked clonal SNVs (%) in female polyps. FIG. 16J contains box plots showing median VAF per monoancestral and polyancestral female polyps (assigned in FIG. 4G). Line shows medina VAF cut-off (<0.2) to assign ancestry in all polyps, including male. FIGS. 16K and 16L show linear model for allele frequency distribution of sub-clonal mutations that can differentiate between neutral (R²≥0.98) and selective (R²<0.98) evolutionary processes in tumor. Here we use SNVs from WES data. These two polyps are assigned as monoancestral and polyancestral using the number of APC mutations in WES data. FIG. 16M show that monoclonal polyps have higher proportion of selective evolution compared to polyclonal polyps. FIG. 16N shows overall, ˜60% of the polyps show selective clonal evolution. FIG. 16O shows ASC cells from three cohorts. FIG. 16P is a volcano plot showing differential gene expression between monoancestral and polyancestral ASC cells. Only a selective list of genes is labeled here. X-axis is truncated for monoancestral ASC. FIG. 16Q shows pathway analysis using DEG shows distinct molecular programs between monoancestral and polyancestral polyps. FIG. 16R shows high CytoTRACE score in monoancestral ASC cells compare to polyancestral ASC cells supports higher stem-like program in monoancestral polyps contributing to proliferative advantage and subsequent clonal selection. FIG. 16S shows expression of canonical stem cell marker LGR5 between two groups.

FIGS. 17A to 17O show tracking clonal composition of spontaneous mouse intestinal tumors. FIG. 17A show hematoxylin and eosin (H&E) staining of Apc^{Min/+}-driven mouse intestinal tumor. This mouse model generates low grade tumors that are equivalent to polyps or precancer. FIGS. 17B and 17C show UMPA embedding of barcoded intestinal tumor cells from NSC-seq experiment. Tumor cell cluster is assigned based on expression of tumor-associated marker genes as shown in the dot plot in FIG. 17C. FIG. 17D shows cell cycle status, CYTOTRACE score, and fetal gene (Marcks1) expression across annotated cell types. FIG. 17E shows clone contribution analysis for CBCs and Tumor cells. FIG. 17F contains box plots representing relative mutation per homing barcodes (hBC) across major annotated cell types. Based on mutation density, EC and Paneth cells are divided into two groups: red (T) and green (N) doted circles. Lineage analysis of EC and Paneth cells subsets with Tumor cells supports tumor-derived Paneth and enterocyte population. FIG. 17G is a heat map representing pairwise barcode mutations correlation for lymphocytes. Peripheral blood lymphocytes are from FIG. 7G and tumor infiltrating lymphocytes are from FIG. 17B. FIG. 17H shows three clones are projected onto the UMAP. FIG. 17I shows differential parent clone fraction for the three representative clones. FIG. 17J contains dot plots represent differential distribution of eRSC score, epiHR score, and coreHRC score across three clones. FIG. 17K shows differential distribution of enterocyte proportion, CytoTRACE score, and iCMS2 score across clones. FIG. 17L is a single-cell lineage tree reconstructed using cells from FIG. 17B. Clones are labeled by same color as in FIG. 17H. FIG. 17M shows whole-exome sequencing (WES) of mouse tumors supports selective evolutionary pressure in mouse tumor. FIG. 17N is a schematic of early embryonic clonal intermix-based ancestry assessment. Some tumors could show presence of mosaic early embryonic mutations, supporting polyancestral composition (right). FIG. 17O is a heat map showing mosaic distribution of early embryonic mutations across regionally distinct tumors and adjacent normal tissues from same mouse using DNA barcode sequencing. Color represents mutant barcode proportion. First four mutations are widely present across tissues, representing their initiation before endoderm development. Four of the five polyancestral tumors (star marks and assigned by the number of Apc mutation) show intermix of multiple early embryonic clonal that are also found in adjacent normal epithelium. This data suggests early intermixing of clones during mouse gut epithelial development and consistent with polyclonal origins of tumors attributed in human colorectal polyps.

FIGS. 18A to 18E show an overview of single-cell pooled CRISPR screens using knockout libraries. FIG. 18A is a single guide RNA (sgRNA) capture schematic for the NSC-seq platform. NSC-seq capture sequence (CS) anneals to 3′-end target sites of sgRNA scaffold. Here, CS contains cellular barcode, UMI, and T7 promoter sequences. An additional sequence is added to the 3′-end of the cDNA during reverse transcription via template switching to enable downstream library amplification. FIG. 18B shows sgRNA capture efficiency by NSC-seq assessed in a bulk experiment and compared with bulk DNA sequencing approach. FIG. 18C is a schematic of single-cell pooled CRISPR screens using NSC-seq platform. Five distinct single-cell screens were performed in this study using conventional knockout vector libraries (bottom). FIG. 18D is a representative UMAP plot showing cell with sgRNA or without sgRNA. FIG. 18E shows representative quantification of the number of sgRNAs detected per cell. Cells with 1 detected sgRNA were used for downstream analysis.

FIGS. 19A to 19C show assessment of sgRNA 469 expression. FIG. 19A is a schematic of WT and truncated sgRNAs (isoform). A representative sgRNA (Dkk1) read alignment and visualization using IGV (bottom) from EpH4 cells with Brie library. FIG. 19B shows fraction of WT and isoform sgRNAs expression across three whole-genome CRISPR knockout libraries from Addgene. FIG. 19C shows predicted number of off-target sites across the mouse genome for variable isoform length.

FIGS. 20A to 20E show assessment of sgRNA detection efficiency and replicate reproducibility. FIG. 20A is an overview of bulk NSC-seq experiment using NSC-seq CS. FIG. 20B shows experimental design of two independent biological replicates to compare sgRNA detection from DNA- and RNA-based reads. FIG. 20C shows Pearson correlation between replicates using DNA- or RNA-based detection. Only sgRNAs that are present in both biological replicates are plotted here. FIG. 20D shows correlation between DNA- and RNA-based sgRNA detection in two replicates. sgRNAs are categorized into two groups, concordant and discordant sgRNAs (DNA reads>100 and RNA reads<10). FIG. 20E show overlapping discordant sgRNAs between two biological replicates. Number of sgRNAs are shown within the diagram.

FIGS. 21A to 21G show analysis of a pooled single-cell gene fitness screen. FIG. 21A shows overview of a gene fitness screen using EpH4 cell line with Brie library. FIG. 21B shows UMAP representation of perturbed EpH4 cells with Leiden clustering (left) and cell cycle status (right). FIG. 21C shows UMAP representation of WT EpH4 cells. FIG. 21D shows top 25 enriched sgRNAs. Many of these sgRNAs are targeting known antiproliferative genes (star mark). FIG. 21E shows expression of a selective gene (Phactr4) in control and targeted cells. FIG. 21F shows detection of essential and non-essential gene-targeting sgRNAs from EpH4 cells with Brie library using bNSC-seq approach. FIG. 21G shows Pearson correlation between the gene regulatory coefficients (FIG. 21B) of each pair of sgRNAs in a linear model using MIMOSCA after adding cell-state and number of unique genes as covariates. Here, sgRNAs are group into modules by similar regulatory effect. Perturbation of similar regulatory programs result in similar transcriptional impact. Regulatory programs for each module listed here is based on GO term analysis. Distinct modules are marked on the left as red line and representative sgRNAs are listed under each module. Similar approaches were used for all other single-cell perturbation analysis in this study.

FIGS. 22A to 22G show analysis of trametinib resistant screen. FIG. 22A shows overview of trametinib resistant screen using SW620 cell line with Brunello library. FIG. 22B shows UMAP representation of trametinib resistant cells with Leiden clusters (left) and cell cycle status (right). FIG. 20C shows Pearson correlation between the regulatory coefficients (B) of top sgRNAs identified two distinct modules (group 1 and group 2), representing ubiquitin and Rho pathways, respectively. FIG. 22D shows differential gene expression analysis between cells in group 1 and group 2. Only significant genes are colored as red (upregulate in group 1) and blue (upregulate in group 2) with LFC+2 and p<0.05. Truncated fold change showed as line break in x-axis. FIG. 22E shows expression of glutathione metabolism gene GGT5 (Gamma-Glutamyltransferase 5) between two groups. FIG. 22F shows pathway analysis using top DEGs between two groups. FIG. 22G shows selective list of genes expression across resistant cells.

FIGS. 233A to 23G show analysis of glutamate pathway gene essentiality screen. FIG. 23A shows overview of gene essentiality screen using MC-38 cell line with custom KO library (n=15 genes, 4 sgRNAs/gene). See method section for custom gene knockout sgRNA library preparation (retrovirus). FIG. 20B shows UMAP representation of MC-38 cells that survived 1 week after viral transduction and cell cycle status (right). FIG. 20C shows distinct regulon activities (SCENIC) across the Leiden clusters. FIG. 23D shows sgRNA frequency across dataset. Undetected sgRNAs (Ctps, Gclc, Gfpt1, Got2, Ppat) are labeled as essential genes (blue) in glutamate pathways. FIG. 23E shows Pearson correlation between the regulatory coefficients (B) of non-essential sgRNAs identified two distinct modules (group 1 and group 2). FIG. 23F shows DEGs between two modules in previous panel. Only significant genes are colored as red (upregulate in group 1) and blue (upregulate in group 2) with LFC+2 and p<0.05. Grik4 (Glutamate receptor, ionotropic, kainate 4) is upregulated in group 2. FIG. 23G shows pathway analysis between these two groups show differential metabolic signatures.

FIGS. 24A to 24K show analysis of cell death pathway screen in mouse primary T cells. FIG. 24A shows overview of cell death pathway screen using Cd8+ cytotoxic T cells (CTLs) with custom KO library. FIG. 24B shows UMAP representation of tumor infiltrating immune cells (left) and cell cycle status (right). Marker gene expression across the Leiden clusters are shown in the bottom as dot plot. FIG. 24C shows gene expression-based cell type annotation revealed macrophages and Cd8+ T cells. Recovered sgRNAs were mostly in CTLs (bottom). FIG. 24D shows expression of immune checkpoint genes between two cell types. FIGS. 24E and 24F show in vivo enriched sgRNA frequency (FIG. 24E) represented as log 2 fold change (FIG. 24F) by comparing in vitro sgRNA frequency. FIG. 24G shows Pearson correlation between the regulatory coefficients (FIG. 24B) of cell death pathway targeting sgRNAs identified two distinct functional modules (group 1 and group 2). FIG. 24H shows DEG between two groups in previous panel. FIG. 24I shows regulon activities (SCENIC) between groups revealed differential proliferation and differentiation activities. FIG. 24J shows CYTOTRACE score showed differential proliferative potential between two groups. FIG. 24K shows intrinsic apoptotic gene signature between groups.

FIGS. 25A to 25l show analysis of immune checkpoint pathway screen in mouse primary T cells. FIG. 25A shows overview of immune checkpoint pathway screen using CTLs with custom KO library. FIG. 25B shows UMAP representation of tumor infiltrating immune cells (left) and cell cycle status (right). FIG. 25C shows marker gene-based immune cell type annotation. FIG. 25D shows in vivo and in vitro enriched sgRNA frequency. FIG. 25E shows representation of in vivo sgRNA enrichment or depletion as log 2 fold change. FIG. 25F shows Pearson correlation between the regulatory coefficients (B) of immune checkpoint pathway targeting sgRNAs identified three distinct functional modules. FIG. 25G to 25I shows differentially expressed genes in CTLs across three groups.

FIGS. 26A to 26C show truncated sgRNAs expression in cells. FIG. 26A shows a few representative sgRNA read (Brie library) alignment and visualization using IGV. Position of base after predicted cut is added to sgRNA name to make them unique. Shown are SEQ ID NOs: 18-19. FIG. 26B shows proportional distribution of first two bases across different isoform lengths in Brie library. FIG. 26C contains schematics to assess gRNA expression using terminal deoxynucleotidyl transferase (TdT).

FIGS. 27A to 27M show large scale assessment of isoform sgRNAs across three whole-genome knockout libraries. FIG. 27A shows isoform sgRNAs in Brie library with five technical replicates. X-axes represent shared isoform sgRNAs across the number of replicates. sgRNAs assigned as isoform if truncated reads are found across three of the five technical replicates. FIG. 27B shows almost 36% of total sgRNAs in Brie library are expressed as both WT and isoform. FIG. 27C shows distribution of isoform reads from previous panel. A small proportion of sgRNAs produced only isoform reads. FIG. 27D shows isoform sgRNAs in Brunello library with three technical replicates (Rep A). sgRNAs assigned as isoform if truncated reads are found across all three technical replicates. FIG. 27E shows almost 37% of total sgRNAs in Brunello library are expressed as both WT and isoform. FIG. 27F shows distribution of isoform reads from previous panel. FIG. 27G to 267I show similar as previous three panels, except independent biological replicate of Brunello library (Rep B). FIG. 26J shows shared isoform between two biological replicates. FIGS. 27K to 26M show isoform sgRNAs in GECKOV2 library.

FIGS. 28A to 28E show gene editing efficiency of isoform sgRNAs. FIG. 28A is an overview of in vitro sgRNA efficiency assessment. FIG. 28B shows only WT sgRNA show targeted DNA double strand breakage in 1 hour reaction time. Custom spacer sequences designed for Gab3 WT (20 bp) and Gab3 isoform (15 bp) from IDT. FIG. 28C shows similar condition as before, except 20 hours reaction time and a few non-specific DNA target was added for both WT and isoform reactions in separate wells. Isoform sgRNA cleaves the target DNA (arrow) with less efficiency compared to WT sgRNA. FIG. 28D shows isoform sgRNA mediated off-target DNA cleavage in 20 hours reaction time. Off-target site predicted using online tool. FIG. 28E shows mean gene expression deviation at pseudo-bulk level from EpH4 cells with Brie library. Cells with isoform sgRNAs showed bigger deviation from control (No sgRNA) compared to WT sgRNAs.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

“Nucleic acid” refers to deoxyribonucleotides (DNA) or ribonucleotides (RNA) and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The word “polynucleotide” refers to a linear sequence of nucleotides. The nucleotides can be ribonucleotides, deoxyribonucleotides, or a mixture of both. Examples of polynucleotides contemplated herein include single and double stranded DNA, single and double stranded RNA (including miRNA), and hybrid molecules having mixtures of single and double stranded DNA and RNA. A “3′-UTR” refers to the 3′-untranslated region of an mRNA that immediately follows the translation stop codon.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

The word “expression” or “expressed” as used herein in reference to a DNA nucleic acid sequence (e.g. a gene) means the transcriptional and/or translational product of that sequence. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion the gene is positioned between two transposon linker sequences that allow insertion into the host genome as well as subsequent excision. “Extracting nucleic acids” refers to processes well known in the art to purify or otherwise separate or isolate a nucleic acid (e.g. RNA or DNA) from a mixture (e.g. a cell lysate).

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids. A “viral plasmid” as used herein refers to a plasmid having a viral origin (e.g. a retrovirus). A “2-micron plasmid” refers to a yeast 2-micron circularized plasmid. A “CEN/ARS plasmid” refers to a plasmid constructed to propagate in two different host species and can include an autonomously replicating sequence and a yeast centromere.

A “delivery vector” as used herein, refers to a plasmid or other polynucleotide that includes the sequences of interest (e.g. a perturbation element-encoding DNA) that is introduced into a cell. Optionally, a delivery vector is a plasmid as described herein. Optionally, a delivery vector is a retroviral delivery vector (e.g. lentirviral). Thus, delivery vectors can be any nucleotide construction used to deliver nucleic acids into cells (e.g., a plasmid), or as part of a general strategy to deliver genes, e.g., as part of recombinant retrovirus or adenovirus (Ram et al. Cancer Res. 53:83-88, (1993)). As used herein, plasmid or viral vectors are agents that transport the disclosed nucleic acids into the cell without degradation and include a promoter yielding expression of the appropriate gene or perturbation element in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector.

The viral vectors can include nucleic acid sequence encoding a marker product. This marker product is used to determine if the gene has been delivered to the cell and once delivered is being expressed. Preferred marker genes are the E. Coli lacZ gene, which encodes B galactosidase, and fluorescent proteins, e.g., Venus fluorescent protein or green fluorescent protein. Optionally, the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure.

Construction of suitable vectors employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

A “cell,” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization. Optionally, the cell is one grown in cell and/or tissue culture.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “modulator” refers to a composition that increases or decreases the level of a target molecule or the function of a target molecule or the physical state of the target of the molecule.

The term “modulate,” “modulation,” or “modulator,” as used with reference to modulating an activity of a target gene or signaling pathway, refers to increasing (e.g., activating, facilitating, enhancing, agonizing, sensitizing, potentiating, or upregulating) or decreasing (e.g., preventing, blocking, inactivating, delaying activation, desensitizing, antagonizing, attenuating, or downregulating) the activity of the target gene or signaling pathway. In some embodiments, a modulator increases the activity of the target gene or signaling pathway, e.g., by at least about 1-fold, 2-fold, 3-fold, 4-fold, 5-fold, 6-fold, 7-fold, 8-fold, 9-fold, 10-fold, 15-fold, 20-fold or more. In some embodiments, a modulator decreases the activity of the target gene or signaling pathway, e.g., by at least about 1-fold, 2-fold, 3-fold, 4-fold, 5-fold, 6-fold, 7-fold, 8-fold, 9-fold, 10-fold, 15-fold, 20-fold or more.

The terms “promoter,” “promoter region,” or “promoter sequence,” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping e.g., with nuclease S1), as well as protein binding domains (consensus sequences) responsible for the binding of RNA polymerase.

A promoter is “constitutive” when the gene being transcribed is expressed (e.g. continually expressed) independent of perturbations in the cellular process.

A “promoter of interest” is a promoter sensitive to perturbations in the cellular process (e.g. binding to components of the cell such as translation factors that form part of the cellular machinery performing the cellular function that is being perturbed by the perturbation factor).

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

A “perturbation element” as used herein refers to a nucleic acid sequence, e.g., a RNA sequence, or a DNA sequence, or a polypeptide sequence, an amino acid sequence, or a protein that interferes with the function of a cell (e.g. through interruption of a signaling pathway, interruption of the localization of proteins or nucleotides, interference of post-translational modifications including phosphorylation, or through changes to degradation rates of target molecules). A “nucleic acid perturbation element” is a perturbation element containing a nucleic acid sequence, e.g., DNA, RNA or a combination thereof. A nucleic acid perturbation element can be an inhibitory nucleic acid, an aptamer, a ribozyme, a triplex forming molecule or an external guide sequence. An “RNA perturbation element” is a perturbation element containing RNA, for example, in a stem loop configuration that is capable of altering or disrupting cellular functions such as transcription factor activation. An RNA perturbation element may be an inhibitory nucleic acid, e.g., shRNA, miRNA, siRNA, or CRISPR guide RNA. Optionally, the RNA perturbation element is an antisense molecule or a ribozyme. A “protein perturbation element” is a perturbation element containing a polypeptide sequence, e.g., an antibody. The protein can be full-length or a variant thereof (e.g. having at least 70%, 80%, 90%, 95%, 96%, 97%, 98%, 99% identity). A protein perturbation element may alter or disrupt cellular functions through protein interactions with other cellular components.

Clustered regularly interspaced short palindromic repeats (CRISPRs) are DNA loci containing short repetitions of base sequences. They are associated with cas genes that code for proteins related to CRISPRs. This system can be used in a manner analogous to RNAi to control gene expression. Typically, the Cas9 protein is used in conjunction with a CRISPR guide RNA that targets a gene of interest and interferes with transcription. More specifically, co-expression of dCas9 and a sgRNA blocks transcription by interfering with transcriptional elongation, RNA polymerase binding, or transcription factor binding. The system is known and has been described in Larson, et al., Nature Protocols 8:2180-2196(2013), and U.S. Publication No. 2014/0068797, which are incorporated by reference herein in their entirety. Thus, the CRISPRi system can be customized for any desired gene of interest.

A “perturbation element-encoding DNA sequence” as used herein is a DNA encoding the RNA sequence or amino acid sequence for a perturbation element.

A “perturbation element identifying DNA sequence” or “DNA barcode” as used herein refers to a sequence of nucleotides uniquely associated with individual perturbation elements and encodes a “perturbation element identifying RNA sequence” enabling identification of active perturbation elements. In some embodiments, the perturbation element identifying RNA sequence is detected using sequencing techniques (e.g. deep sequencing).

A “perturbation element identifying RNA sequence” or “RNA barcode” as used herein refers to a sequence of nucleotides uniquely associated with individual perturbation elements and enables identification of active perturbation elements. Optionally, the perturbation element identifying RNA sequence is detected using sequencing techniques (e.g. deep sequencing).

The term “an amount of” in reference to a polynucleotide or polypeptide, refers to an amount of a component or element is detected. The amount may be measured against a control, for example, wherein an increased level of a RNA barcode following exposure to a perturbation element in relation to its corresponding perturbation element identifying DNA sequence demonstrates enrichment of the barcode. In contrast, a decreased level of a RNA barcode following exposure to a perturbation element in relation to its corresponding perturbation element identifying DNA sequence demonstrates non-enrichment of the barcode. The amount of a component may be decreased through such pathways as proteasomal degradation or increased through such pathways as enhanced promoter activity. The measure of enrichment or non-enrichment may be performed with pooled cells. The amount may be a frequency count of the number of RNA barcodes.

The term “cellular barcode” refers to a nucleic acid sequence unique to each cell.

The term “unique molecular identifier (UMI)” refers to a nucleic acid sequence unique to each transcript, including gRNA.

### NSC-Seq

RNA sequencing (RNA-seq) is a genomic approach for the detection and quantitative analysis of messenger RNA molecules in a biological sample and is useful for studying cellular responses. Single-cell RNA sequencing (scRNA-seq) is a version of this that allows researchers to study the transcriptomes of individual cells. The first, and most important, step in conducting scRNA-seq has been the effective isolation of viable, single cells from the tissue of interest. Next, isolated individual cells are lysed to allow capture of as many RNA molecules as possible. For polyadenylated mRNA molecules, poly [T]-primers are commonly used for capture. Analysis of non-polyadenylated mRNAs (e.g. gRNAs) is typically more challenging and requires specialized protocols or libraries.

Many of such approaches, such as Perturb-seq, utilize specialized vectors that allow the indirect capture of sgRNAs by standard 3′-end single-cell RNA-seq (scRNA-seq) methods. Direct 3′ sgRNA methods have recently been reported but they also require custom modification of plasmid libraries. The use of these specialized vectors limits the scale and flexibility of genetic screens, since they are incompatible with existing genomes-scale knockout (KO) libraries. Moreover, specialized vectors are susceptible to sgRNA-barcode swapping events due to lentiviral template switching.

Disclosed herein is a custom 3′ single-cell capture platform, called Native sgRNA Capture and sequencing (NSC-seq), that enables flexible and multi-purpose single-cell CRISPR screening using existing KO vector libraries.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Temporal Recording 1 of Mammalian Development and Precancer

### Introduction

Mammalian development originating from a fertilized egg (zygote) is a remarkable process, comprising a highly orchestrated series of cell divisions and lineage diversifications. Tumorigenesis shares cellular and molecular events with embryonic development, many of which have been recently appreciated [Grisendi, S., et al., Nature, 2005. 437(7055):147-53; Kaiser, S., et al., Genome Biol, 2007. 8(7): R131; Aiello, N. M. et al. Dis Model Mech, 2016. 9(2):105-14; Ma, Y., et al., J Cell Mol Med, 2010. 14(12):2697-701; Bellacosa, A., Am J Med Genet A, 2013. 161a (11):2788-96]. The molecular mechanisms underpinning these events remain the central questions in cancer biology. Fundamental to understanding these mechanisms is the knowledge of their origins and temporal ordering [Visvader, J. E., Nature, 2011. 469(7330):314-22; Hoadley, K. A., et al., Cell, 2018. 173(2):291-304.e6; Tanay, A., et al. Nature, 2017. 541(7637):331-338; Jassim, A., et al., Nature Reviews Cancer, 2023. 23(10):710-724]. Previous work utilized non-reversible genetic alterations in tumors, such as mutations and copy number changes, in either bulk or spatially resolved sequencing to track temporal events [Sprouffske, K., et al. Cancer Prev Res (Phila), 2011. 4(7):1135-44; Heiser, C. N., et al., bioRxiv, 2023:2023.03.09.530832; de Bruin, E. C., et al., Science, 2014. 346(6206):251-6; Gerstung, M., et al., Nature, 2020. 578(7793):122-128; Pan-cancer analysis of whole genomes. Nature, 2020. 578(7793):82-93]. While these analyses are applicable to human tumor studies, they provide only inferences of chronological order or only monitor clonality, lacking the precision to track associated cellular events.

Recent barcoding strategies in mammalian system [Chan, M. M., et al., Nature, 2019. 570(7759):77-82; Bowling, S., et al., Cell, 2020. 181(6):1410-1422.e27], when combined with single-cell sequencing, have shown promise in unraveling the origins and chronological sequence of cellular events. However, their potential for recording temporal events long term is constrained by limited barcode diversity [Wagner, D. E. et al., Nat Rev Genet, 2020. 21(7):410-427] and loss of information due to large deletion of multiple adjacent cut-sites [Byrne, S. M., et al., Nucleic Acids Res, 2015. 43(3): e21; Shin, H. Y., et al., Nat Commun, 2017. 8:15464]. We are curious about the potential of a multimodal framework, pairing human single-cell multi-omics data with long term temporal tracking in mice, for addressing questions regarding cellular origins. For this, we generated one of the largest multi-omic atlasing datasets on human sporadic polyps to date. Furthermore, we developed a custom multi-purpose single-cell platform called Native sgRNA Capture and sequencing (NSC-seq) for simultaneous capturing of mRNAs and gRNAs, that can leverage self-mutating CRISPR barcodes [Perli, S. D., et al. Science, 2016. 353(6304); Kalhor, R., et al., Science, 2018. 361(6405); Kalhor, R., et al. Nat Methods, 2017. 14(2):195-200] in mouse to enable lineage tracking and temporal recording using accumulative mutation patterns. We utilized NSC-seq to validate canonical developmental branching during mouse gastrulation and developed new insights into the timing of tissue diversification, undefined routes of cellular differentiation, and novel embryonic progenitor cell populations. Paired analysis of human atlasing data in conjunction with mice as a validation platform revealed the polyancestral origins of human colorectal tumorigenesis. Our multimodal framework employing natural genetic changes in human paired with induced genetic changes in the mouse illuminate complexities of cellular origins and temporal transitions, and their significance in tumorigenesis.

### Methods

### DNA Barcode Amplification, Library Preparation, and Sequencing:

Barcode loci (hgRNAs) were amplified from genomic DNA (gDNA) templates and library was prepared for Illumina sequencing using previously reported two step PCR amplification protocol [Kalhor, R., et al., Science, 2018. 361(6405)]. Briefly, the first step of PCR was done in real-time PCR reaction and was stopped in mid-exponential phase. The second PCR step was to add Illumina sequencing adapter after diluting the PCR product from first step as templet. Finally, the PCR amplified product was gel purified (˜450 bp) using QIAquick Gel Extraction kit. Sequencing libraries were quantified on a NanoDrop. Duel indexed libraries were pooled and loaded onto Illumina NovaSeq 6000 S4 flow cell using a PE150 kit.

Mutation Recovery from hgRNA:

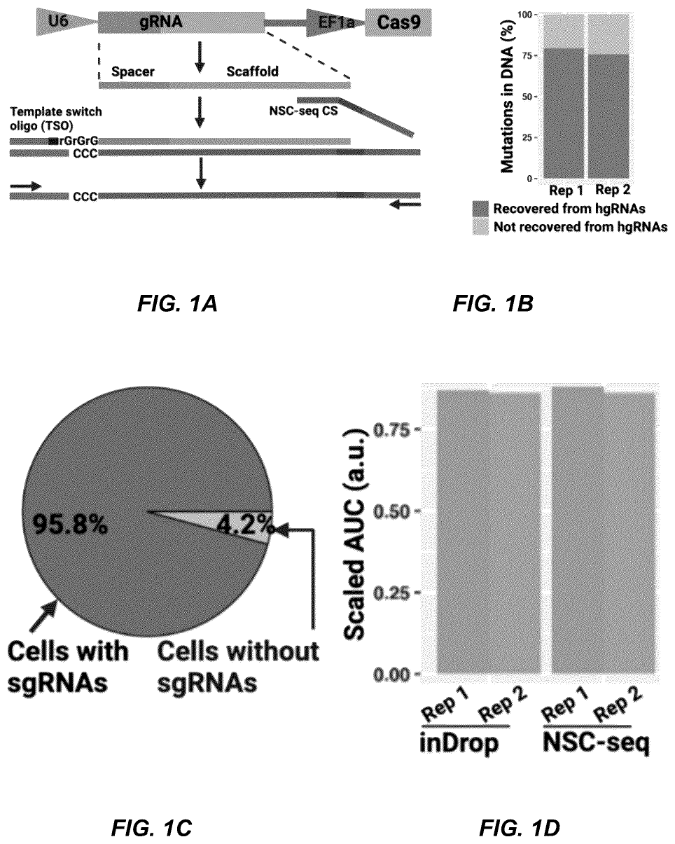

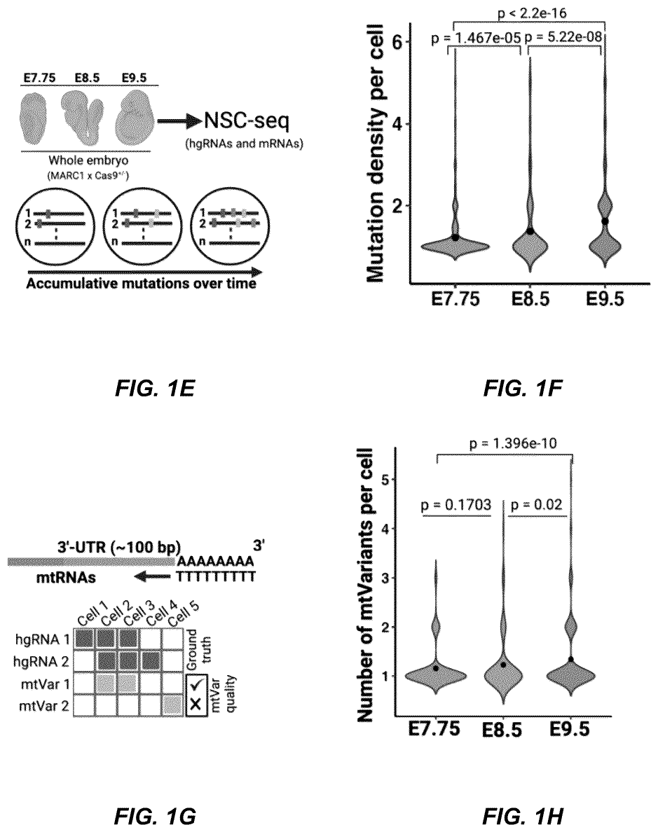

Barcoded HEK293FT cell line was generated using previously reported approach [Kalhor, R., et al. Nat Methods, 2017. 14(2):195-200] with modification. Briefly, lentiviruses were packaged in HEK293T cells for Cas9 plasmid (Addgene: 73310) using a second-generation system with VSV.G as the envelope protein, followed by drug selection. The hgRNA vectors were constructed by incorporating gBlock (IDT DNA), synthesized DNA fragments, into piggyBac vector backbone (Addgene: 104536, 104537). The gBlocks were corresponded to 5 unique homing guide RNAs (BC6, BC17, BC44, BC52, and BC56) with all 21 bp length. The hgRNA vectors were electroporated in HEK293FT-Cas9 cells, followed by drug selection to make barcoded HEK293FT-Cas9-hgRNAs cell line. Barcoded cell line was split into two of a 6-well plate and grown for a week. The plate wells were dissociated using trypsin after 1 week and divided half for bulk DNA extraction and other half for bulk RNA extraction from both wells using kits (QIAGEN). Bulk DNA barcode libraries were prepared using reported before [Kalhor, R., et al., Science, 2018. 361(6405)]. Bulk RNA libraries were prepared using NSC-seq capture sequence as primer and were using similar approaches as NSC-seq library preparation as reported later. All libraries were sequenced using NovaSeq6000 and pair-end sequencing. Reads from bulk DNA libraries were filtered out if there were less than 10 copies detected to remove PCR and sequencing artifact. Due to less sequencing depth in bulk RNA libraries, filtering cutoff was reduced to less than 2 reads. Mutations were called from all the reads using similar approach as reported later. Finally, the proportion of DNA mutations that were also found in RNA libraries was calculated for both wells and represented as bar stack plot (FIG. 1B). Note that, bulk DNA and bulk RNA were not extracted from the same cells, as there was not a simultaneously extraction from the same lysate, rather progenitor population that might impact relatively low mutation detection from bulk RNA libraries.

Native gRNA Capture:

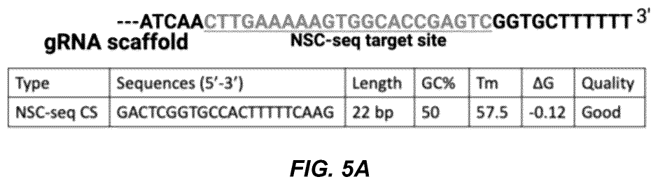

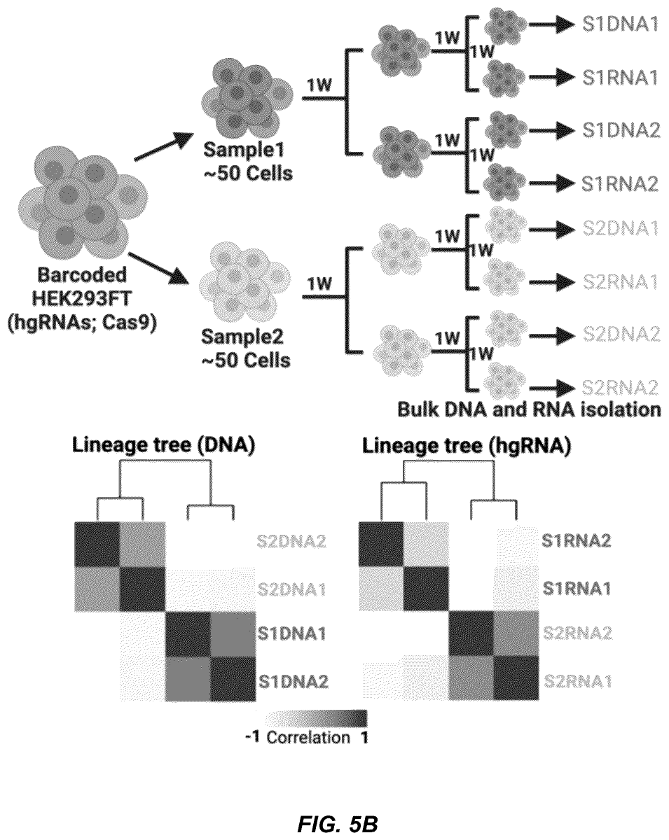

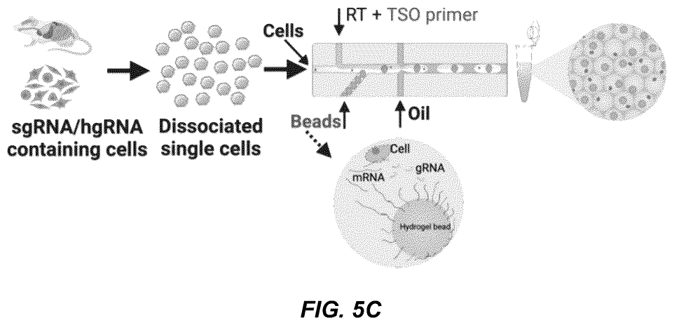

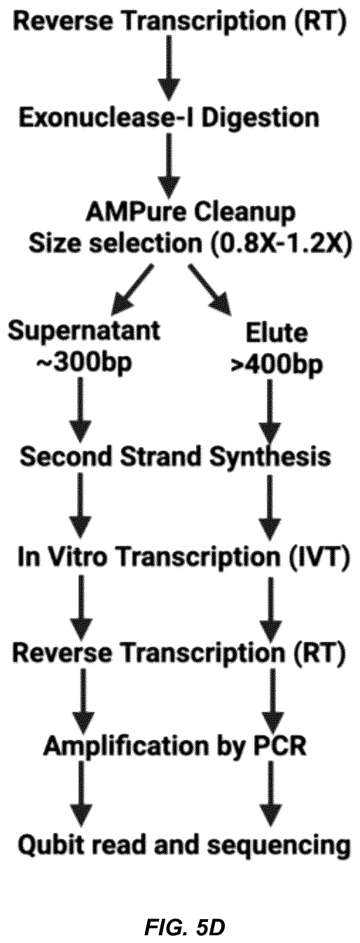

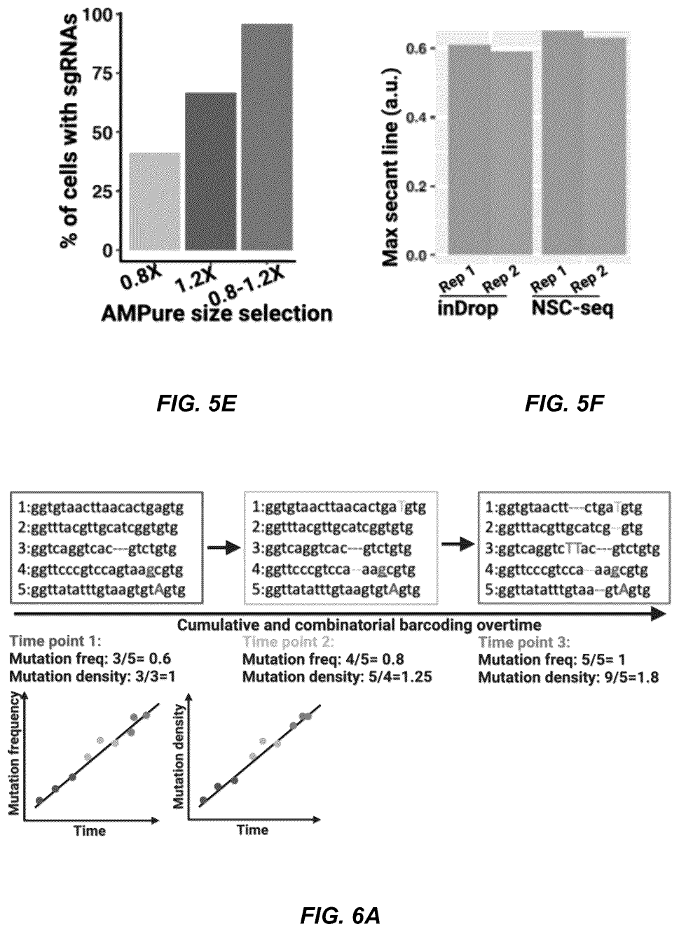

To capture CRISPR guide RNA (gRNA), we designed a 22-bp NSC-seq capture sequence (CS) that is complementary to the 3′-end of the gRNA scaffold. This CS served as a primer to generate cDNAs (FIG. 5A). The 5′-end of gRNAs is variable (for sgRNA, hgRNA, or stgRNA) and accumulate mutations for hgRNA, and thus, does not contain sequences that can be used as a primer to amplify cDNAs. To overcome this technical challenge, we leveraged a unique reverse transcriptase activity called template switching that can append a sequence of interest to the 5′-ends of cDNAs. These primer pairs enabled us to amplify cDNAs that are then followed by sequencing library generation (FIG. 1A). Several initial quality control experiments were conducted to demonstrate the validity of this approach. First, hgRNA barcoded HEK293FT cell line were generated that also express constitutive Cas9 using reported approach with modification. The gDNA mutation recovery rate was calculated from hgRNAs and found that almost 80% of the gDNA mutations can be recovered using our approach (FIG. 1B). It was found that the captured hgRNAs from barcoded HEK293FT cells can infer similar lineage tree as gDNA-based tree, imply that hgRNAs carry lineage defining mutational information (FIG. 5B). Secord, we designed custom hydrogel beads using inDrop v2 chemistry that contain two types of capture sequence primers, PolyT (70%) for regular polyadenylated mRNA capture and NSC-seq CS (30%) for gRNA capture, followed by optimized NSC-seq workflow (FIGS. 5C, 5D). We found that 70:30 ration gives us better gRNA capture efficiency, without compromising mRNA capture. Third, we evaluated the gRNA capture efficiency using EpH4 cell line that contain Brie (Addgene: 73633) whole-genome knockout (KO) plasmid library. After optimizing cDNA size selection, gRNA capture efficiency found up to ˜95% (FIGS. 1C, 5E). Fourth, the transcriptome capture efficiency of NSC-seq beads was estimated by comparing inDrop beads and found similar capture efficiency (FIGS. 1D, 5F). Finally, NSC-seq approach was applied on multiple embryonic time points, fetal and adult intestinal epithelium, intestinal tumor, and single-cell perturbation experiment (companion study) to recover hgRNAs/sgRNA and mRNAs simultaneously and found high quality transcriptome, in addition to gRNA recovery. Thus, NSC-seq platform allows a robust gRNA capture efficiency without compromising transcriptome quality.

### Mouse Embryo Collection and Single Cell Dissociation:

MARC1 mouse [Kalhor, R., et al., Science, 2018. 361(6405)] was mated with Rosa26-Cas9^{+/+} mouse [4] in a 12-h light/dark cycle mouse house and embryonic days (E) was calculated based on vaginal plug at noon considered to be at E0.5. Post-implantation embryos were dissected in cold PBS and staged by Theiler's criteria. Collected embryos were incubated for ˜20 mins at 37° C. with Accumax (Millipore Sigma, #A7089) supplemented with DNase (Life technologies, #AM2222; 1:1,000 dilution ratio), vigorous pipetting was done during incubation for further disaggregation until complete dissociation was visually confirmed. Dissociated cells were further filtered through into a new 1.5 ml Eppendorf tube and several time spin down for 5 mins at 700 rpm on a tabletop centrifuge (4° C.). Finally, dissociated cells were run into in-house inDrop-based custom droplet capture platform.

### Mouse Intestinal Epithelium and Intestinal Tumor Dissociation:

Small intestinal epithelial tissue samples and resected tumor tissue were collected in 1×DPBS and incubated in chelation buffer (4 mM EDTA) at 4° C. for 1 h. Tissues were dissociated into crypt and villi section by shaking in cold DPBS [Banerjee, A., et al., Gastroenterology, 2020. 159(6):2101-2115.e5; Chen, B., et al., Cell, 2021. 184(26):6262-6280.e26]. Tissue suspension was filtered using 100 uM filter to enrich crypt cells in normal epithelium tissues, followed by three times wash and spin down for 5 mins at 1200 rpm on a tabletop centrifuge (4° C.). Final crypt-enriched tissue precipitate was suspended in TrypLE (Gibco, #12604013) and incubated for at 37° C. for 3 mins to dissociate into single cells. Then, enzyme was neutralized using serum containing FACS buffer and three times wash in DPBS and spin down for 5 mins at 1200 rpm on a tabletop centrifuge (4° C.).

### Single-Cell NSC-Seq Platform Optimization:

Single cell NSC-seq experiment was performed using the inDrops microfluidic platform [Klein, A. M., et al., Cell, 2015. 161(5):1187-1201] as described in (Star Protocols [Simmons, A. J., et al., STAR Protoc, 2022. 3(3):101570]) with modifications to accommodate the capture of guide RNA. In brief, cells were co-encapsulated into droplets with custom barcoded beads. Custom beads were designed in collaboration with companies (1cellbio and RAN biotechnologies) that contain NSC-seq capture sequence (30%) and PolyT (70%). RT/Lysis buffer was modified to add 2.5 mM template-switch oligo (TSO) primer. Reverse transcription was carried out at 50° C. for 1 hour, followed by demulsification and storage of barcoded cDNA at −80° C. Transcriptomic sequencing libraries were prepared as described before [Southard-Smith, A. N., et al., BMC Genomics, 2020. 21(1):456], with modifications in the first SPRI purification to yield a second cDNA pre-library enriched for barcoded cDNAs (hgRNAs) between 250 and 350 bp (0.8×-1.2× double AMPure size selection). hgRNA libraries were processed similar to transcriptomic libraries, except fragmentation. Next, the hgRNA libraries were PCR amplified using indexing primers (8 cycle) and gel purified (˜270 bp band) using QIAquick Gel Extraction kit. Sequencing libraries were quantified on an Agilent Bioanalyzer. Duel indexed libraries were pooled and loaded onto Illumina NovaSeq 6000 S4 flow cell using a PE150 kit targeting of 100 million reads for transcriptome libraries and 50 million reads for barcode libraries [Chen, B., et al., Cell, 2021. 184(26):6262-6280.e26].

gRNA Capture Efficiency:

Mouse mammary epithelial cells (EpH4) were transduced by Brie whole-genome knockout (KO) lentivirus plasmid library (Addgene: 73633) using previously reported approach [Doench, J. G., et al., Nat Biotechnol, 2016. 34(2):184-191]. Transduced cells were drug selected, followed by droplet-based encapsulation using NSC-seq platform. Three encapsulated fractions were processed, and library prepared using different AM Pure (Beckman) size selection approach to identify best gRNA recovery approach (FIG. 5E). Double size selection (0.8×-1.2×) and two independent library preparation approach enabled more than 95% gRNA recovery rate (FIG. 1C). All single-cell NSC-seq libraries in this study were prepared using this double size selection approach.

mRNA Capture Efficiency:

EpH4 cell line was used to compare relative transcriptome capture efficiency between NSC-seq beads and inDrop beads in duplicate scRNA-seq libraries (FIG. 1D, 5F). As described before [Arceneaux, D., et al., iScience, 2023. 26(7):107242], scaled AUC and maximum secant line metrics are the measurement of scRNA-seq data quality. The AUC metrics is the ratio between the area under the cumulative transcript count curve and the area of the minimal rectangle that circumscribes the curve. Secant line distances for a dataset is defined as the vertical distances between the cumulative transcript count and the diagonal of the curve for each barcode in the dataset. Scaled AUC and maximum secant line values are generated using the formatted_figures( ) function from the plot_quality_score python module available at the AmbientContaminationMetrics github repository. The maximal secant line distance for a dataset was used as a metric that takes a higher value for datasets with less ambient gene contamination. Both values take a range between 0 and 1. Higher values of the metrics indicate a dataset with less ambient gene contamination.

### Somatic Mitochondrial Variants Detection:

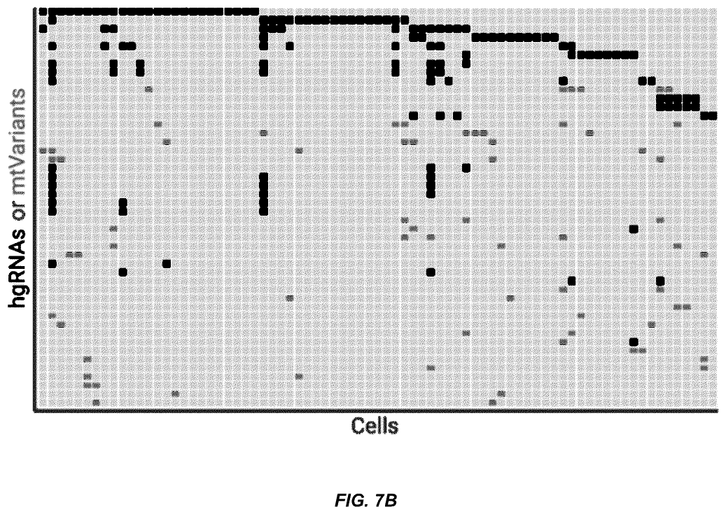

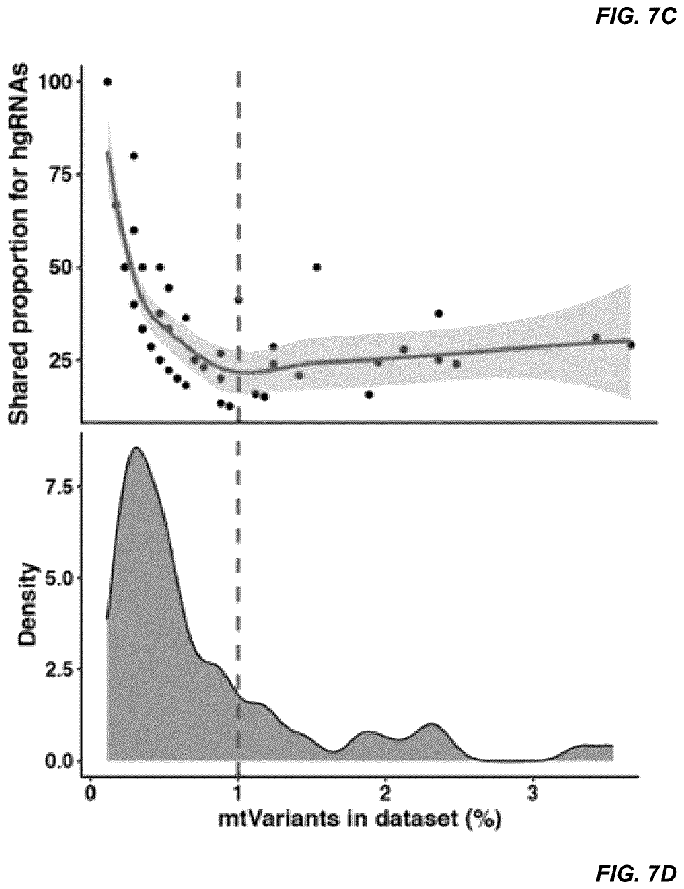

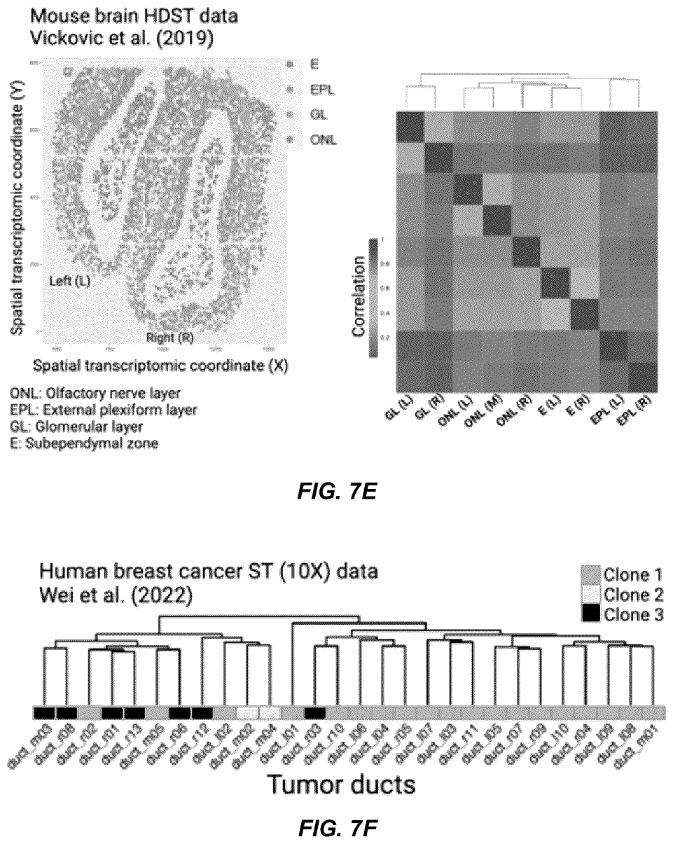

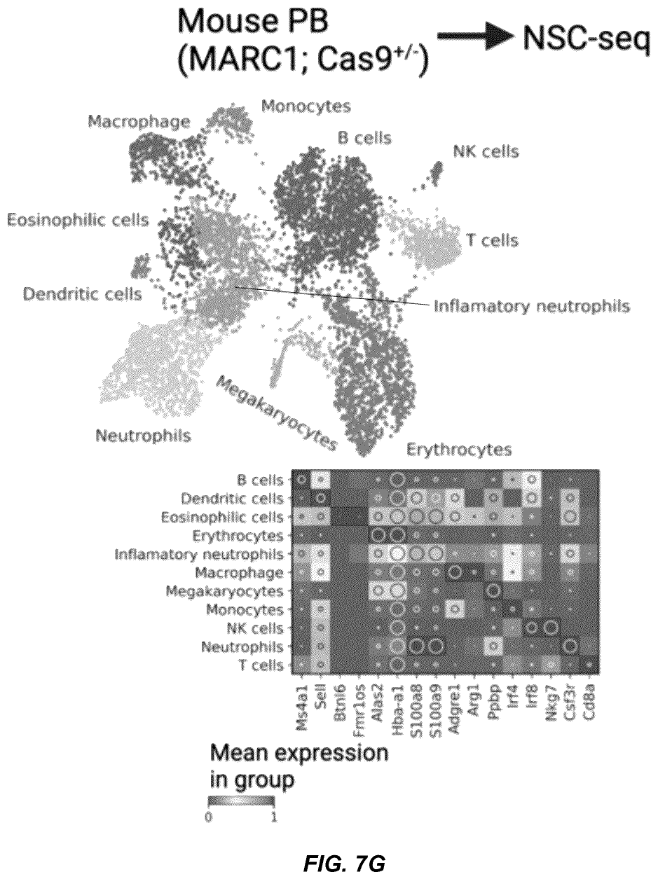

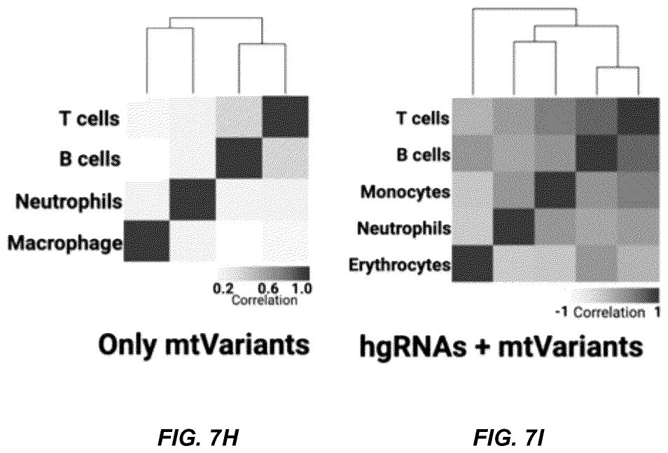

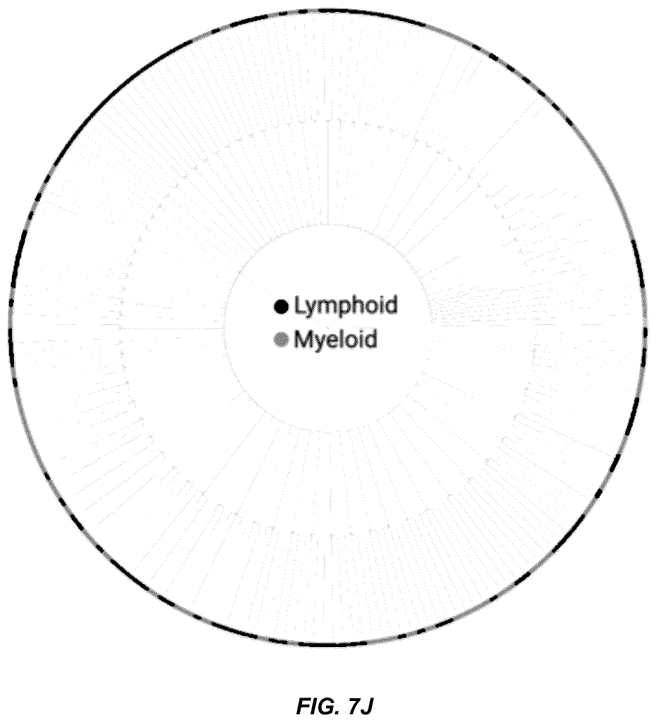

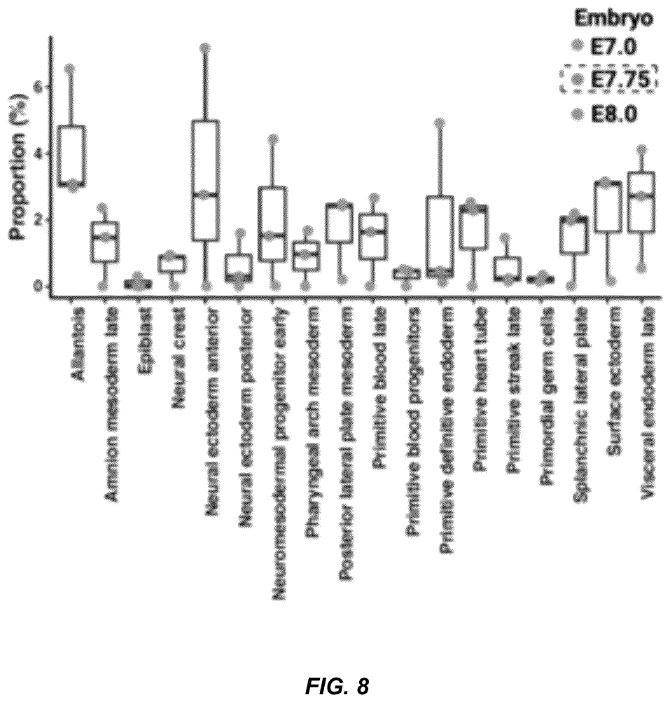

Somatic mitochondrial variants (mtVars) were called from 12 mitochondrial mRNA (mtRNA) reads that were present in our NSC-seq libraries (FIG. 5D). Recently, mtVars have been demonstrated as natural genetic barcodes for tracking cell lineages (FIG. 7A) [Ludwig, L. S., et al., Cell, 2019. 176(6):1325-1339.e22; Xu, J., et al., Elife, 2019. 8]. As mitochondria does not have proof-reading activities, mitochondrial DNA (mtDNA) accumulates 10-100× more somatic mutations compared to gDNA [Kang, E., et al., Cell Stem Cell, 2016. 18(5):625-36; Stewart, J. B. et al. Nat Rev Genet, 2015. 16(9):530-42]. However, the ability to detect enough high-quality lineage informative mtVars from droplet-based scRNA-seq dataset is still inadequate, required whole genome sequencing or special enrichment protocol [Miller, T. E., et al., Nat Biotechnol, 2022. 40(7):1030-1034], limiting application of mtVars for lineage tracking in mammalian development and disease. As mitochondrial 3′-UTR regions are widely captured by polyT primers in scRNA-seq and 150 bp paired-end reads were sequenced by using Illumina NovaSeq 6000 platform, around 100 bp from 3′-end of mtRNA reads were used as natural genetic barcodes. Due to hundreds of independent mitochondrial genomes per cells (heteroplasmy), pairwise read alignment approach [Pagès, H., et al., R package version, 2019. 2(0):10.18129; Kent, W. J., et al. Genome Res, 2002. 12(4):656-64] were used to detect the rare somatic mtVars. Several approaches were taken to filter out PCR and/or sequencing errors and to identify lineage informative mtVars (see GitHub for more details). First, custom scripts were used to extract high quality paired-end reads from fastq files, followed by trimming, alignment, and variant calling. Private variants, not shared with any other cells in the dataset, were removed from downstream analysis. Second, a ‘germline mtVars bank’ was built after combining data from all the samples (mixed mice background from MARC1 and Cas9 cross) to filter out germline mtVars. Third, hgRNA mutations were used as ‘ground truth’ lineage markers to set up a threshold for true somatic mtVars (FIG. 7B-7C). Finally, coincidental mtVars were also determined by the deviation from the linear relationship between mtVars frequency and cell frequency and filtered out from the data. Next, lineage defining power of the mtVars was tested using few reported spatial transcriptomics datasets. Adult mouse brain HDST data [Vickovic, S., et al., Nat Methods, 2019. 16(10):987-990] were used to call mtVars, followed by pseudo-bulk lineage analysis of left and right side of multiple brain layers (FIG. 7E). Left side of each brain layer is closely related to the right side of corresponding layer, implied that brain neuronal layer development is earlier than left-right axis commitment [Kiecker, C. et al. Annu Rev Neurosci, 2012. 35:347-67]. Similarly, human breast cancer ST data were used to call mtVars, followed by clonal relationship analysis among duct regions. The close relationship between clone 2 and 3 compared to clone 1, supported the clonal relationship determined by CNV analysis. Although the number of somatic mtVats increase over embryonic time points, mtVars were not used for temporal recording approach due to less sensitivity over increasing time points. Overall, these data support that the pipeline could detect highquality somatic mtVars that could complement hgRNA barcode mutations to increase the information content of the NSC-seq experiment, enabling high fidelity lineage-tracking of single cells.

Gremlin mtVars Bank:

After mutation calling from mitochondrial reads, all mtVars were aggregated from multiple independent mice to generate a germline mtVars bank. The barcoded mice were mixed background, cross between MARC1 mice [1] and Cas9 mice [Platt, R. J., et al., Cell, 2014. 159(2):440-55]. Several initial quality control experiments were conducted to demonstrate the germline mtVars. First, the mtVars were filtered out based on the single cell ID that didn't pass the single-cell quality thresholds. Second, the mtVars those were present in all independent mouse samples were initially assigned as germline mtVars. Finally, the germline mtVars were further confirmed by their high prevalence in ambient/empty droplets (determined by ranked gene count using scanpy [Wolf, F. A., et al. Genome Biol, 2018. 19(1):15]). Thus, the list of germline mtVars was generated (github table) and all the data were filtered out using this list for lineage analysis.

### Barcode Alignment and Mutation Calling:

In-house python and R scripts were used to analyze barcode sequencing data with modification from previous report [Kalhor, R., et al., Science, 2018. 361(6405)]. Briefly, hgRNA barcode (spacer) sequences were extracted from paired-end reads using 12 bp constant sequence from gRNA scaffold region and the reads from R1 and R2 were mapped using read headers. Single cell barcode IDs were extracted from R1 whereas hgRNA barcodes were extracted from R2. Barcode reads (hgRNAs) were trimmed in between TSO sequence and 10 bp upstream of gRNA scaffold. UMI sequences (6-bases) were extracted from TSO reads to identify PCR duplication. First 8 bases of the hgRNA reads were being used to assign barcode ID, as the hgRNAs usually do not accumulate mutations on this region. Barcode reads were discarded if barcode ID assignment was unsuccessfully. Wild type barcodes were also removed for downstream analysis, as they do not carry any lineage information. Reads were aligned to custom reference genome (WT barcode sequence) that enabled identification of Cas9 target sites [Kent, W. J., et al. Genome Res, 2002. 12(4):656-64; Höijer, I., et al., Genome Biol, 2020. 21(1):290]. Cas9-induced mutations were detected using reported approach [Pagès, H., et al., R package version, 2019. 2(0):10.18129] on an in-house server. Similar approaches were also used for bulk DNA barcode or bulk hgRNA sequencing analysis (see GitHub for more details).

### Single-Cell Lineage Tree Reconstruction:

Data preprocessing: For the reconstruction of lineage trees, we considered three different types of cells—a) cells for which both homing barcode and gene expression data were available, b) cells for which both mitochondrial barcode and gene expression data were available, and c) cells for which homing barcode, mitochondrial barcode and gene expression data were available. For both homing and mitochondrial barcodes, the cells that contained at least one mutation were selected. The mutations that were present in only one cell were removed as they did not harbor any lineage information. For mitochondrial barcodes, germline mtVars were further filtered out. For each selected cell, a complete mutation profile of length mₕ+mₘ was constructed by appending homing and mitochondrial barcodes, where mₕ and mₘ denote the number of selected unique homing and mitochondrial mutations respectively in the dataset. For a cell missing a specific type of lineage barcode, a zero vector of length either mₕ (for cells missing homing barcode) or mₘ (for cells missing mitochondrial barcode) was appended. Following the data preprocessing of LinTIMaT [Zafar, H., et al. Nat Commun, 2020. 11(1):3055], the scRNA-seq data for each cell was denoised and imputed using DrImpute [Gong, W., et al., BMC Bioinformatics, 2018. 19(1):220], and imputed gene expression profiles were normalized and log-transformed using the Scanpy library [Wolf, F. A., et al. Genome Biol, 2018. 19(1):15]. Reconstruction of larger trees: For larger datasets consisting of more than 2000 cells, we adopted a greedy approach in reconstructing the trees. First, we performed principal component analysis (PCA) of the complete mutation profiles and based on 30 PCs, the cells were clustered into different groups which differed in terms of their mutation profiles. A kNN graph was constructed based on cell-cell distance (computed based on 30 PCs) matrix and graph-based clustering was performed using Leiden algorithm from the Scanpy library. These clusters essentially represented groups of cells with distinct mutation profiles where some mutations were shared by most of the cells in a cluster (thus carrying the lineage information) and the set of such lineage-defining mutations differed across different clusters. Thus, each cluster represented a subtree in the complete lineage and the subtree topology was reconstructed based on the mutation profiles of the cells within the cluster representing the subtree. The subtree topology for each cluster was reconstructed using LinTIMaT. The root of each subtree was identified based on the location of a completely unmutated profile that was manually added along with the cells within the cluster. To connect the subtrees to construct the complete single-cell lineage tree, a mutation frequency profile was computed for each cluster. The mutation frequency for each mutation was computed by measuring the proportion of cells within the cluster carrying that particular mutation. To compute the mutation frequency profiles, the mutations present in 10 or more cells (considering the complete dataset) were considered as the goal was to reconstruct the early lineage branches that should harbor mutations occurring relatively early in the experiment and thus shared by more cells. The mutation frequency profile of each cluster was binarized to denote the presence or absence of a mutation in a cluster. LinTIMaT was used for reconstructing a tree topology (also called a skeleton tree) from the binarized mutation profiles of the clusters where the leaves represented the clusters. The root of the skeleton tree was again identified based on the location of a manually added completely unmutated profile. The subtree topologies reconstructed for each cluster were attached back to the respective leaves of the skeleton tree to construct the complete single-cell lineage tree where each leaf represented a single-cell and was colored according to their tissue type/cell type. The unmutated profiles were removed from each subtree. To reconstruct the skeleton tree, LinTIMaT was run only with the mutation likelihood optimization until convergence. Reconstruction of smaller trees: For smaller datasets consisting of <1000 cells, we directly applied LinTIMaT on the complete mutation profile of the cells to reconstruct the single-cell lineage tree. LinTIMaT was run till the convergence of the mutation likelihood and for the set of cells with identical mutation profile, gene expression likelihood was optimized for delineating the branching.

### Pseudo-Bulk Lineage Tree:

The proportion of total single cells in a tissue type that carry a particular mutation was calculated using in-house R scripts. A ‘mutation×tissue type’ matrix was generated using the calculated mutation proportion. The matrix was filtered our if a mutation was present in only a single tissue type in the matrix. Similarly, if a mutation was present in all the tissue types in the matrix was also filtered out. A pairwise Manhattan distance matrix was generated using mutation proportion matrix as reported before [Kalhor, R., et al., Science, 2018. 361(6405)]. Finally, the resulting distance matrix was clustered hierarchically using Ward's clustering criterion to obtain a tree [Murtagh, F. et al. Journal of classification, 2014. 31:274-295].

### Temporal Recording:

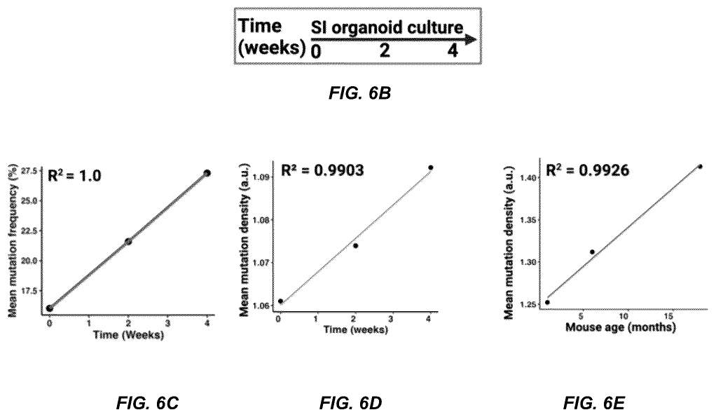

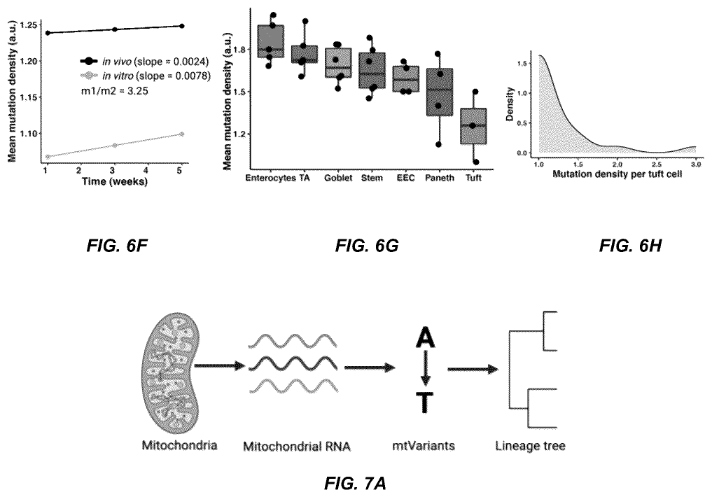

CRISPR system with self-mutating gRNA (hgRNA/stgRNA) [Kalhor, R., et al., Science, 2018. 361(6405); Kalhor, R., et al. Nat Methods, 2017. 14(2):195-200; Perli, S. D., et al. Science, 2016. 353(6304)] has been used to develop a ‘clock’ like system that can longitudinally record temporal events as a form of indelible indel mutations in DNA, which are accumulative and increase at a steady rate (FIG. 6A). Two mutational features were identified in self-mutating CRISPR system: (i) mutation frequency (ratio of mutated vs. wildtype barcodes) and (ii) mutation density (average number of mutations in barcodes) that theoretically track with time and can be leveraged to record temporal information at single-cell resolution. To validate this hypothesis, intestinal organoids were generated from an adult barcoded mouse (MARC1; Cas9+/−) and continuously cultured for over a month (see organoid culture approach below). During this culture period, the organoids were subsampled every two weeks and bulk DNA sequencing was performed to assess mutations in genomic barcodes. Indeed, linear increase of both mutation frequency and mutation density were noticed (FIGS. 8B-8D). A recent study also reported similar observations about mutation frequency in cell lines [Park, J., et al., Cell, 2021. 184(4):1047-1063.e23]. However, mutation frequency is not compatible with singlecell-level temporal recording, as only a limited number of barcodes can be detected per single cell due to dropout and contributes to unacceptably low data quality. To overcome this limitation, the mutation density metric was found to be highly sensitive when used in temporal recording at single-cell scale (FIG. 1E). Next, several approaches were taken to validate the temporal recording using mutation density metric. First, calculation of mutation density at three time points of adult mouse at single-cell resolution resulted in consistent increase of mutation density over time (FIG. 6E). In addition, mutation density for each distinct cell type was consistent with its recognized turnover rate (FIG. 6G-6H). Secord, high cell division rate in organoid culture condition compared to in vivo intestinal epithelium was recorded using mutation density metric. Third, mutation density increased significantly in blood samples over a month period compared to brain samples, implied cell division dependent feature of mutation density. Finally, Cas9 expression from Rosa26 loci and non-homologues end joining (NHEJ) activity were found to be unbiased to any cell type in early embryonic time point, minimizing the potential confounders that might impact the temporal recording. These data support the mutation density as an accurate metric to track temporal events like relative cell division at single-cell resolution using NSC-seq.

### Mutation Frequency and Mutation Density in Bulk:

Mutation frequency (mutated vs. wild type barcodes) was calculated for each of the detected barcode ID. The barcode IDs were filtered out if it was out of range (5-95%), as reported before [Park, J., et al., Cell, 2021. 184(4):1047-1063.e23]. In addition, the barcode IDs were also filtered our if a barcode read count is less than 5% of sample reads and only found in a single sample. Geometric mean of the mutation frequency of all the barcode IDs was calculated and plotted as a scatter plot (FIG. 6C). Next, mean mutation density for each mutated barcode was calculated. The barcode IDs were sorted using similar approached as mutation frequency. Geometric mean of the mutation density was calculated per time point and also plotted as scatter plot (FIG. 6D).

### Mutation Density for Single Cell:

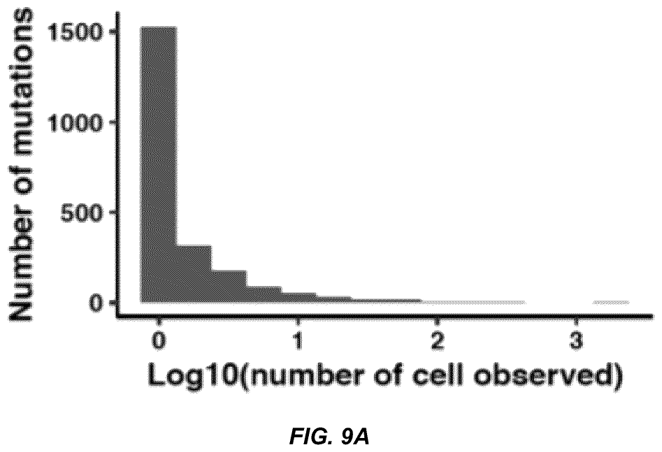

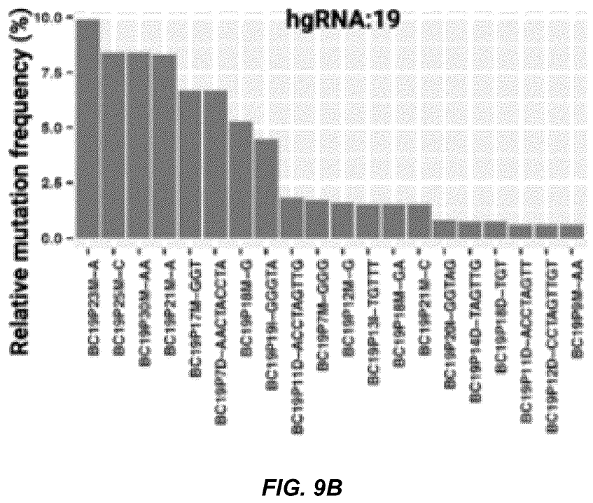

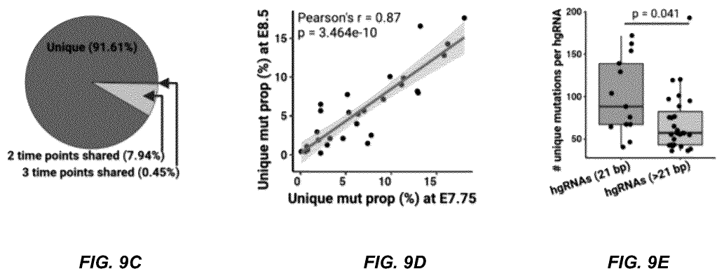

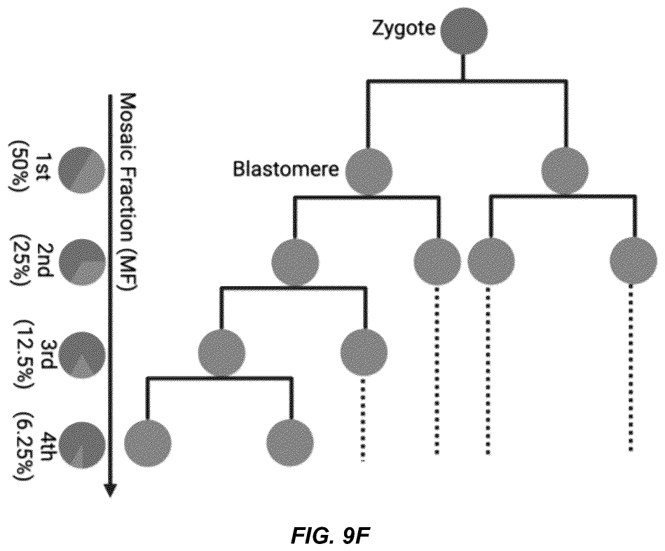

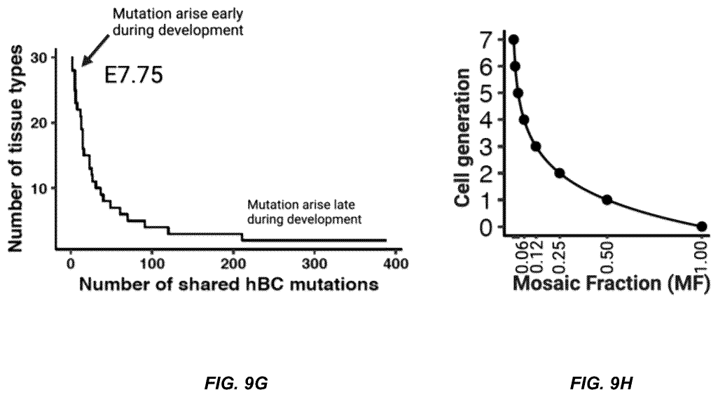

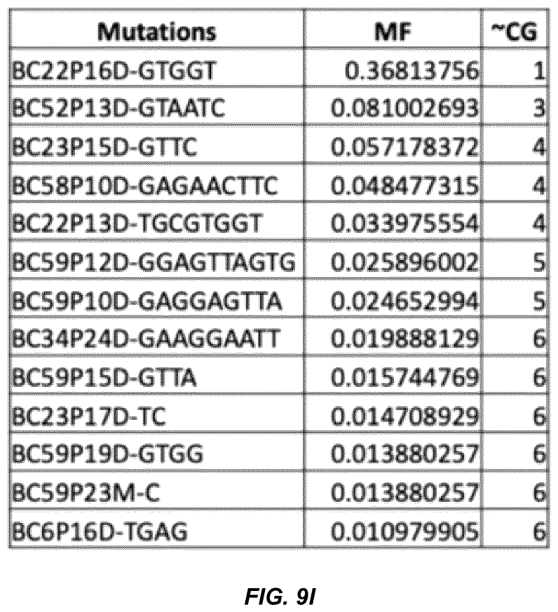

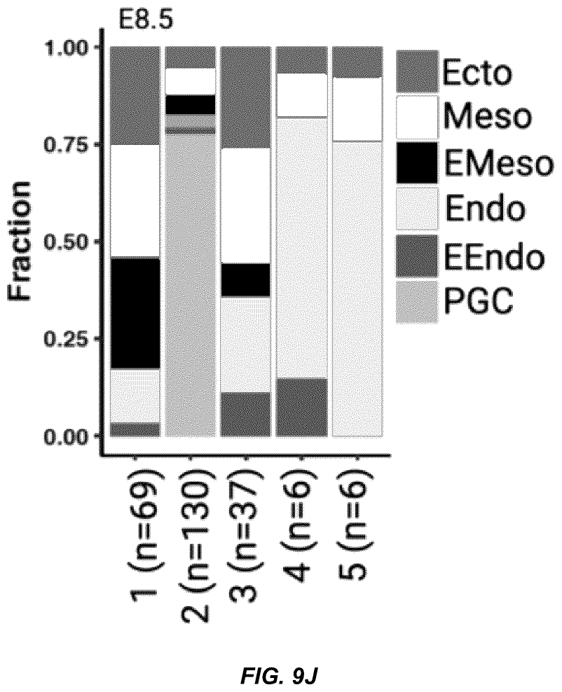

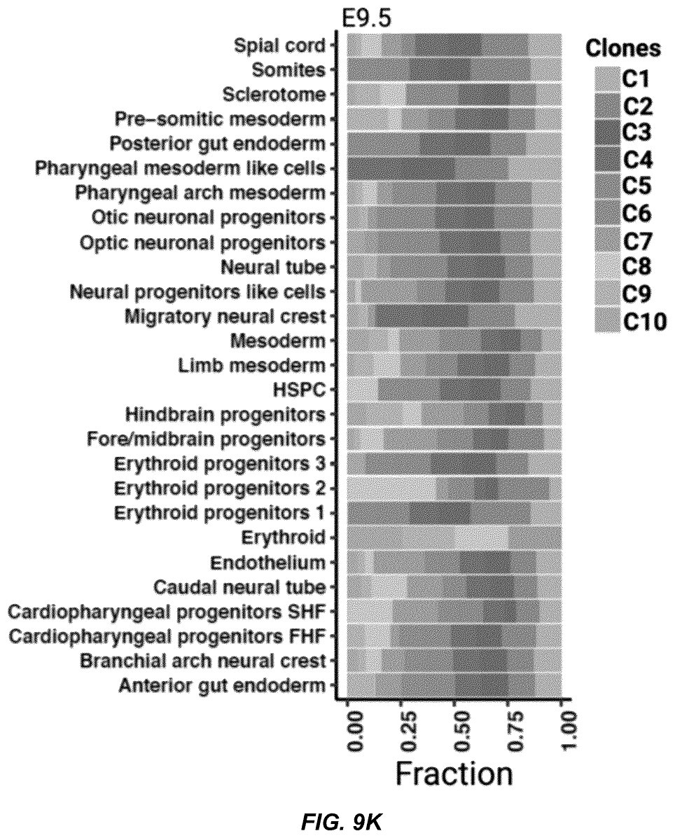

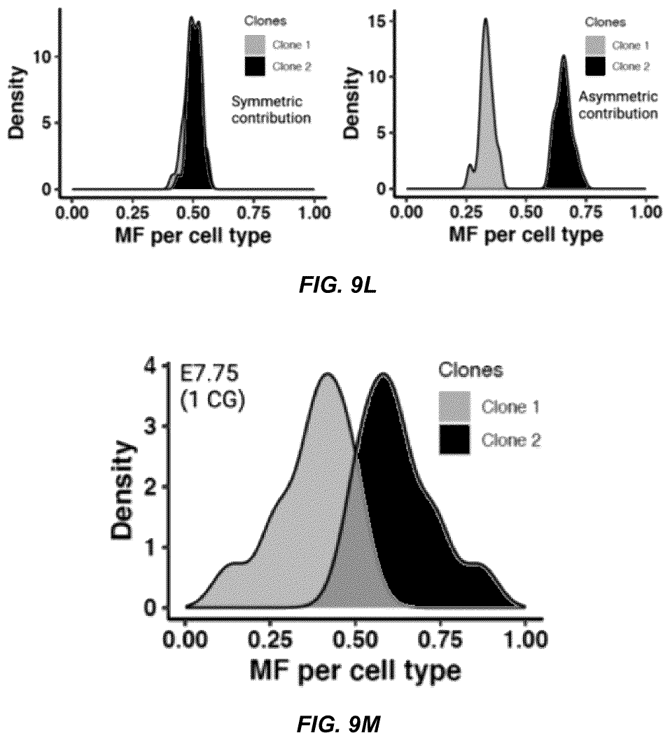

The number of unique mutations per mutated barcode were calculated for all the hgRNA reads after filtering. The mean number of mutations per barcode per cell was calculated using custom R scripts. Outlier cells were detected based on distribution of the mean (mutation density) across the single cell and removed from downstream analysis. Geometric mean of the mutation density was calculated for the remaining single cells per embryonic tissue type (FIG. 1F). Note that, different hgRNAs in MARC1 mouse have different rate of mutation accumulation due to corresponding spacer length (FIG. 9E). Every barcoded mouse/embryo acquires a unique set of hgRNAs with distinct mutation accumulation properties that is partly responsible for creating an unequal mutation density unite across independent mouse/embryo. It is more reliable to compare mutation density among tissue types within a particular mouse/embryo, rather across independent mouse/embryo (with distinct set of hgRNAs). Due to this unequal set of hgRNAs accusation in mice, mutation density score has not been converted into a numeric cell division score, rather using mutation density as arbitrary unite (a.u.) for comparative cell division difference among tissue types within a mouse/embryo.

### Cas9-Induced Mutation Quality Assessment:

To differentiate between the uniquely marked number of cells by an indel mutation versus the independent coincidental mutation due to Cas9 cleavage, the frequency distribution was calculated across three embryonic time points. Coincidentally occurred mutations can be determined from the deviation of linear correlation of indel mutation frequency and observed cell frequency as reported before [Chan, M. M., et al., Nature, 2019. 570(7759):77-82; Bowling, S., et al., Cell, 2020. 181(6):1410-1422.e27]. In general, only a small proportion of indels are showing deviation, supporting relatively rare coincidental indel mutations due to Cas9 cleavage as reported before. Moreover, we calculated the shared and unique mutations across three embryos and found that less than 1% of indels are shared among three embryos, supporting relatively rare coincidental events.

### Early Embryonic Mutation and Mosaic Fraction:

Early embryonic mutation (EEM) was defined by the presence of a mutation across all three major germ layers (ectoderm, mesoderm, and endoderm) [28]. EEMs were found to be usually distributed across >50% of annotated embryonic tissue types. The concept of mosaic fraction (MF) was taken from allele frequency in bulk tissue sequencing [Behjati, S., et al., Nature, 2014. 513(7518):422-425; Ju, Y. S., et al., Nature, 2017. 543(7647):714-718]. In case of single cell, MF was defined by the proportion of cells carrying a particular mutation in a dataset. The number of cells with an EEMs was divided by the total single cells captured per embryonic time point. Note that, due to detection limit and dropout in single cells, calculated MF was probably an underestimation. Thus, MF-based cell generation (CG) was assigned at the next closest stage (FIGS. 9H-91).

### Clonal Contribution and Clone Size Analysis:

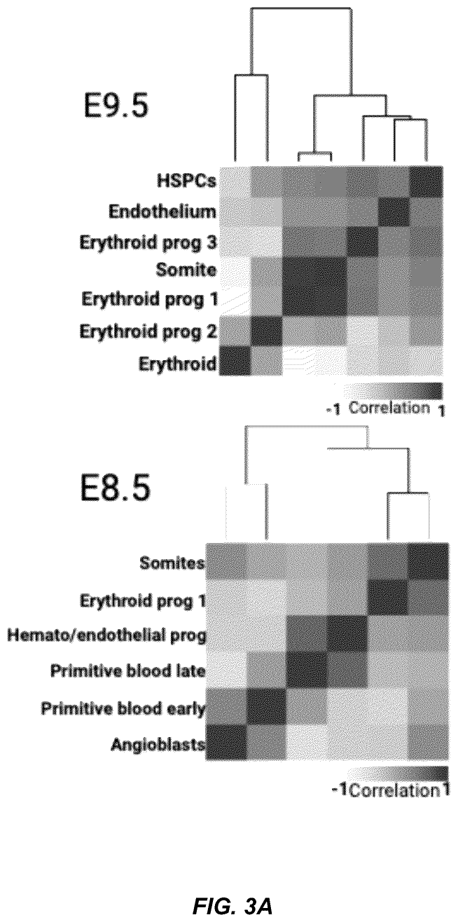

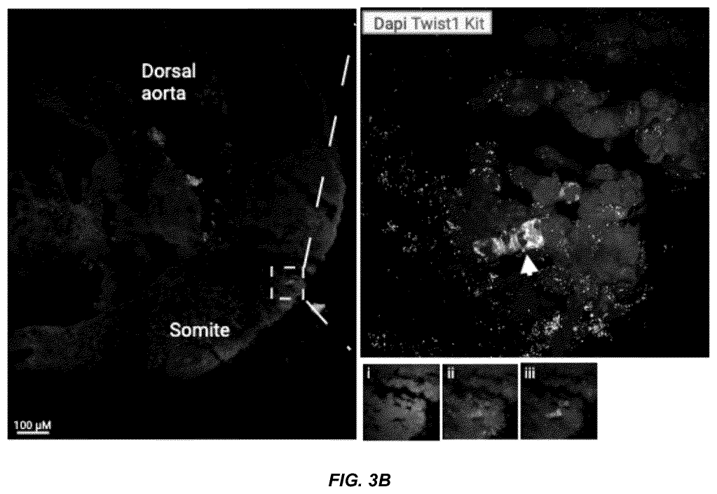

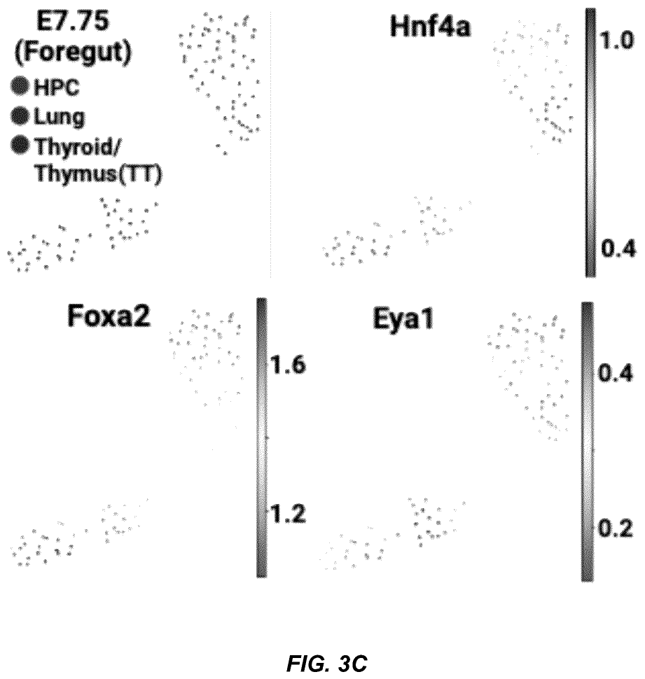

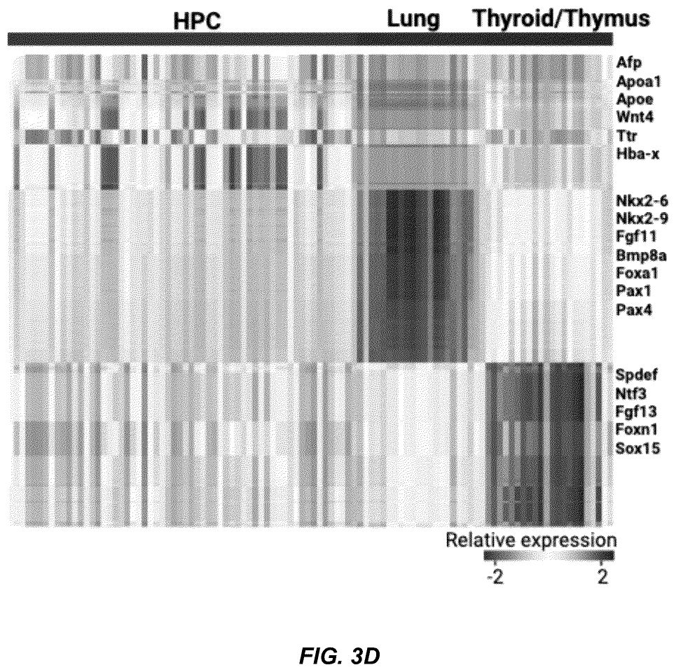

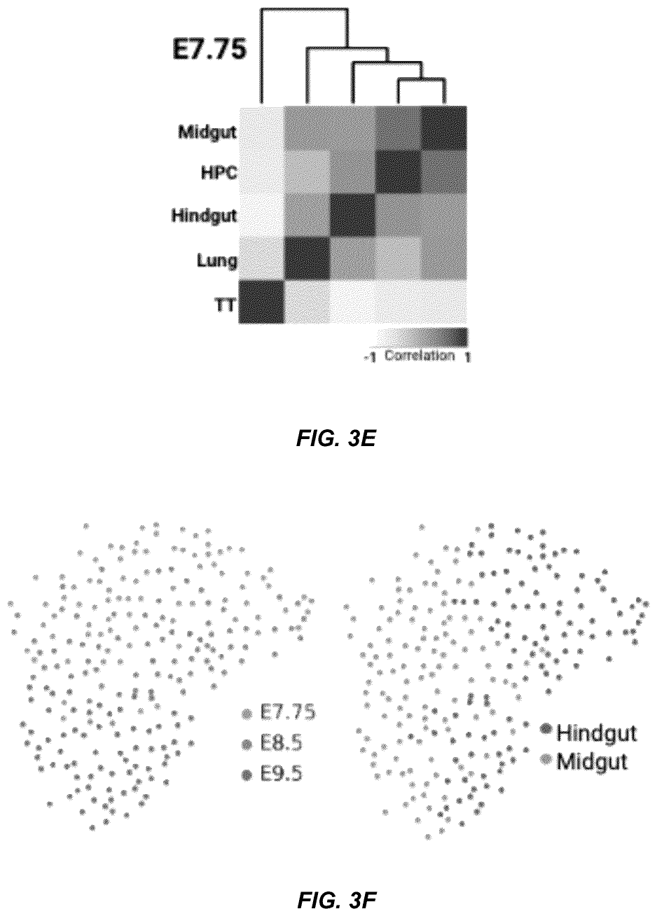

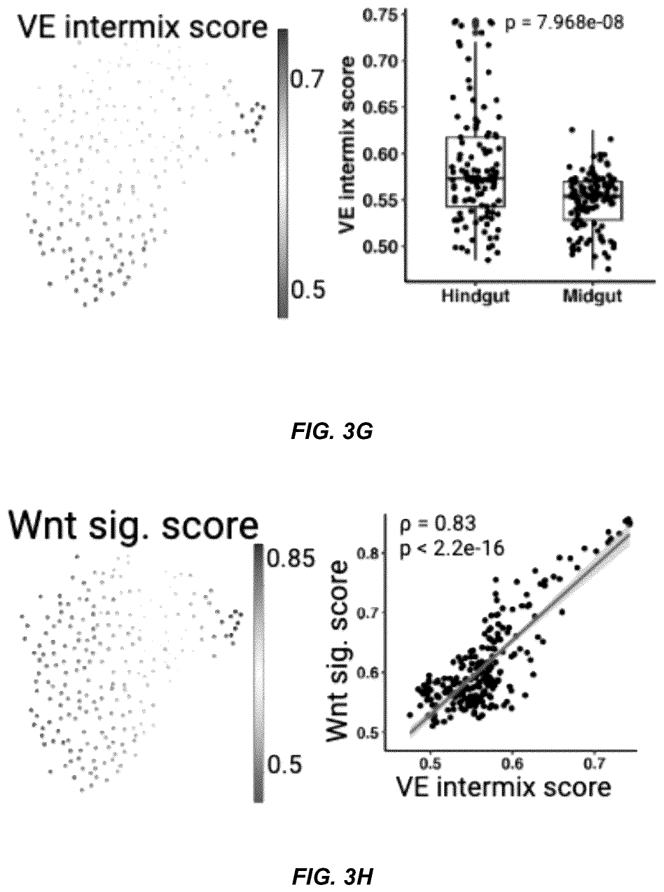

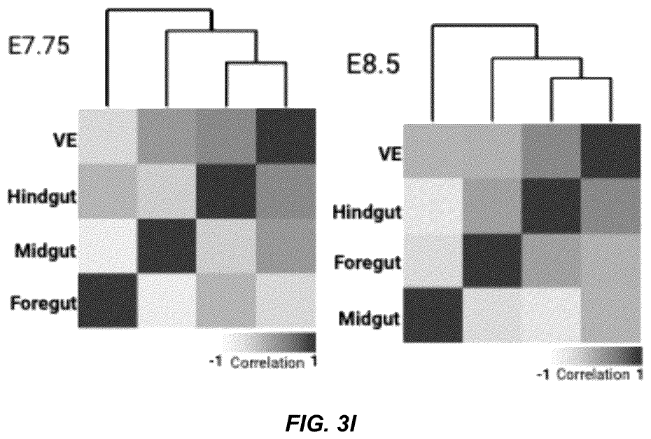

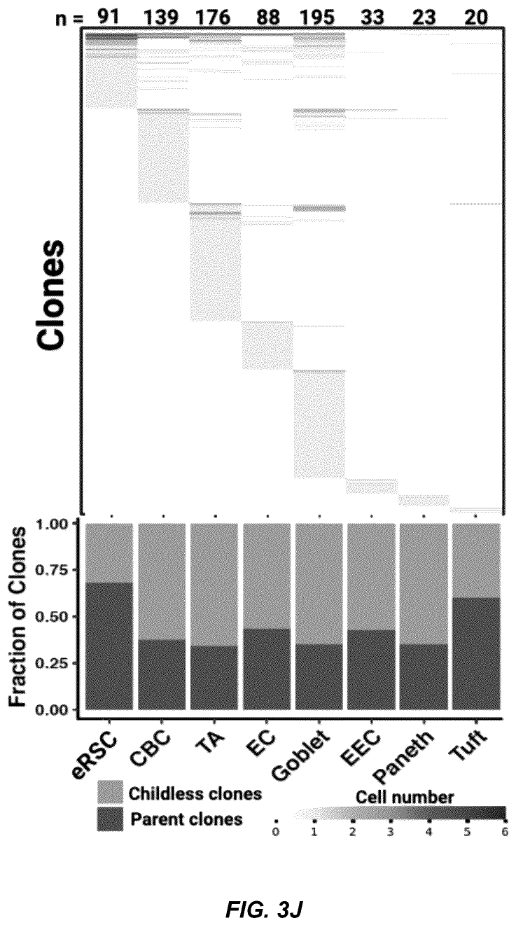

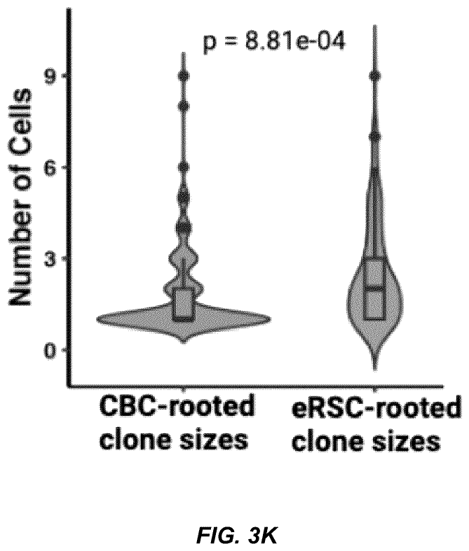

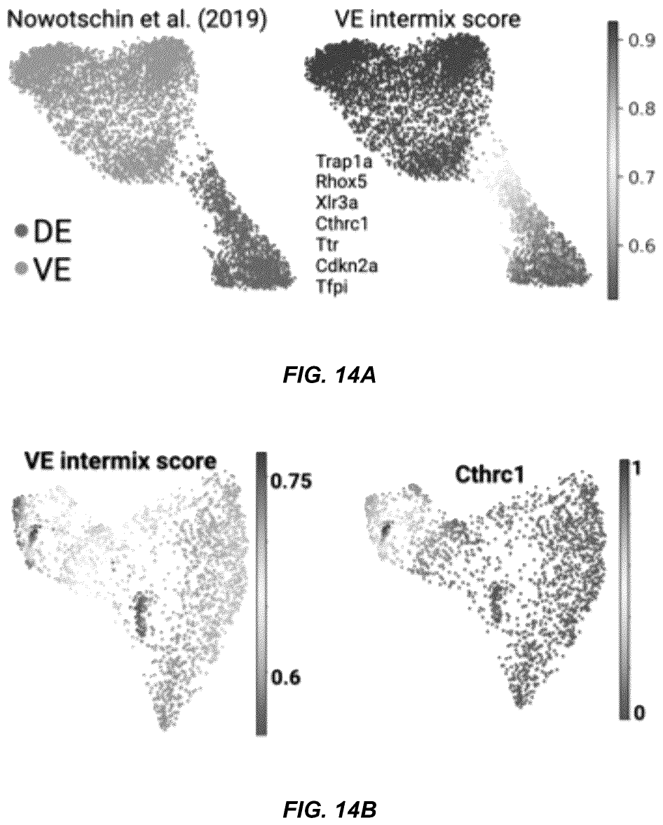

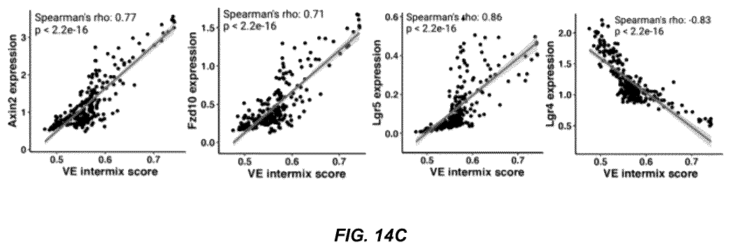

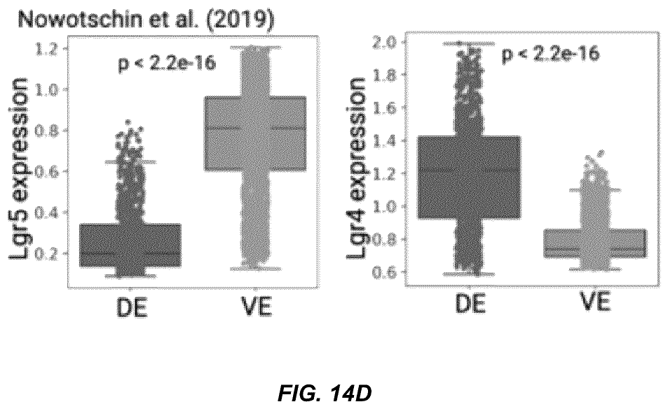

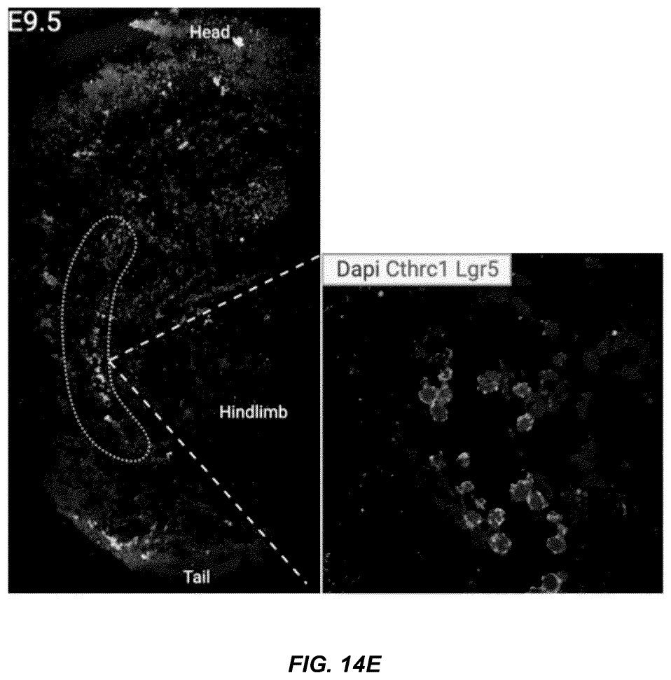

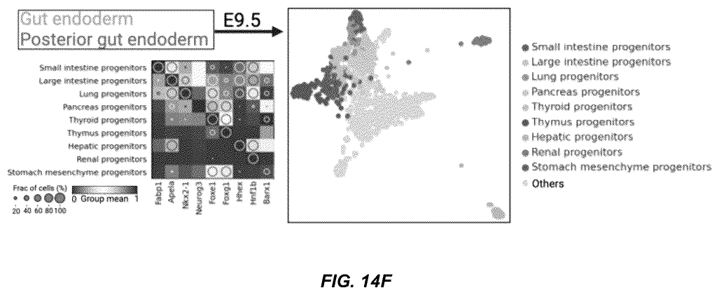

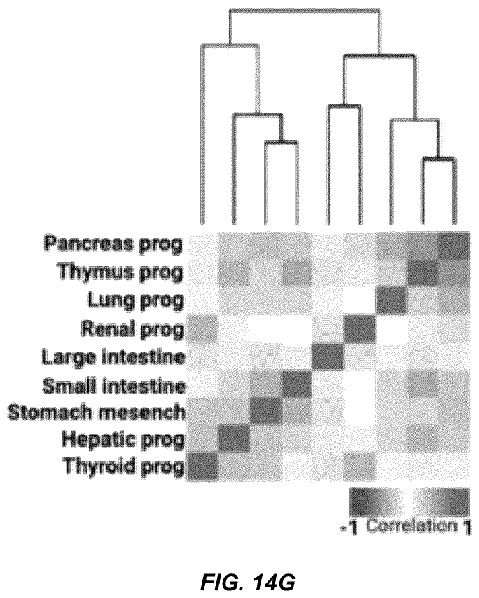

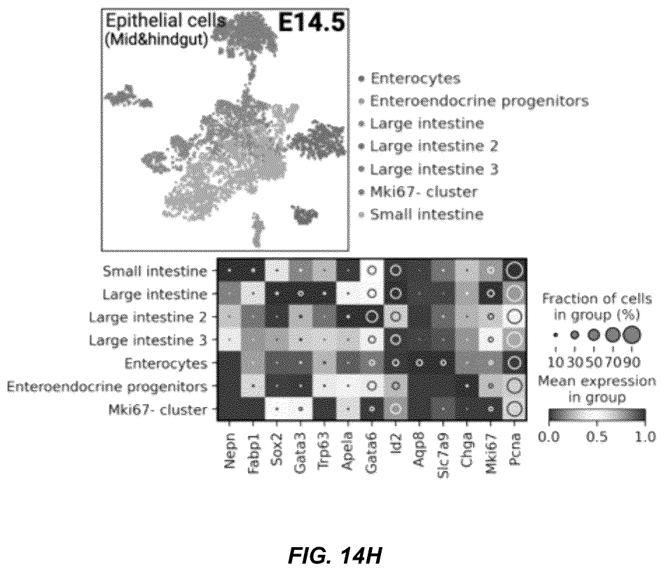

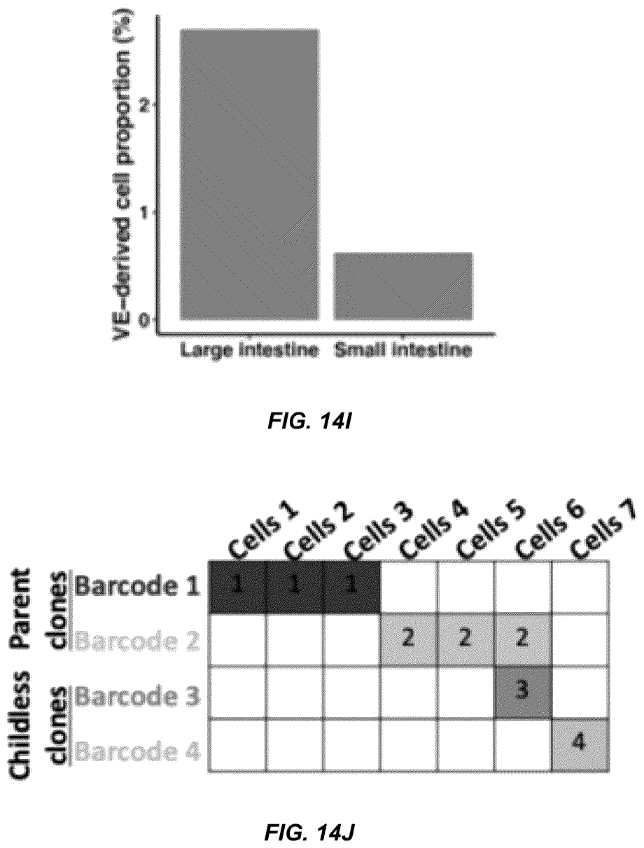

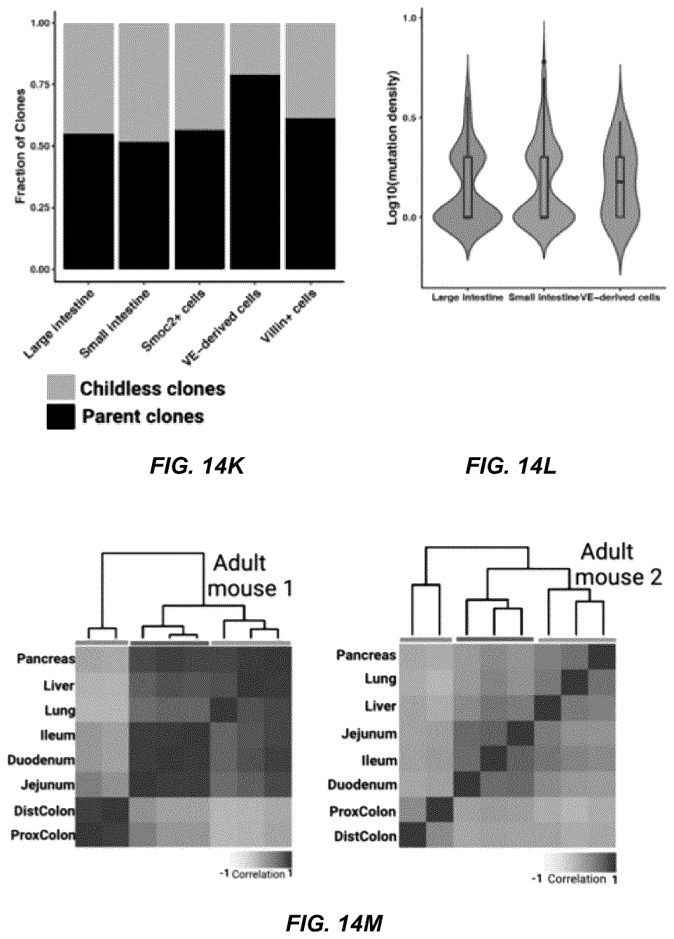

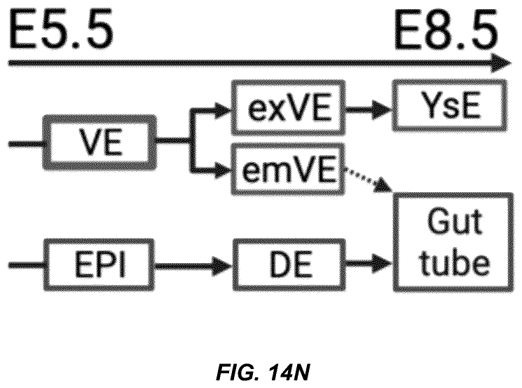

Barcode mutation based clonal contribution analysis was carried out as previously described [Bowling, S., et al., Cell, 2020. 181(6):1410-1422.e27] but with modifications (FIG. 14J). Briefly, a ‘cell×mutation’ matrix was generated for each dataset. If a mutation (refer to as clone) was present in more than one cells in the matrix, it was marked as parent clone. Whereas, if a mutation was present in only one cells in the matrix, it was marked as childless clone. At a given time, the ratio of parent clone vs childless clone per cell type is an indicator of relative contribution of that cell type. For clone size calculation, a ‘mutaitonxcell’ matrix and calculated the number of cells that shared a particular mutation. If the value is 1, which means only a single cell and no other cells shared, we removed those mutations. Next, the remaining values were plotted as violin plot that correspond to a particulate cell-type (CBC/eRSC)-rooted clone size (FIG. 3L). Bigger clone size means higher contribution.

### Phylogenetic Distance Calculation:

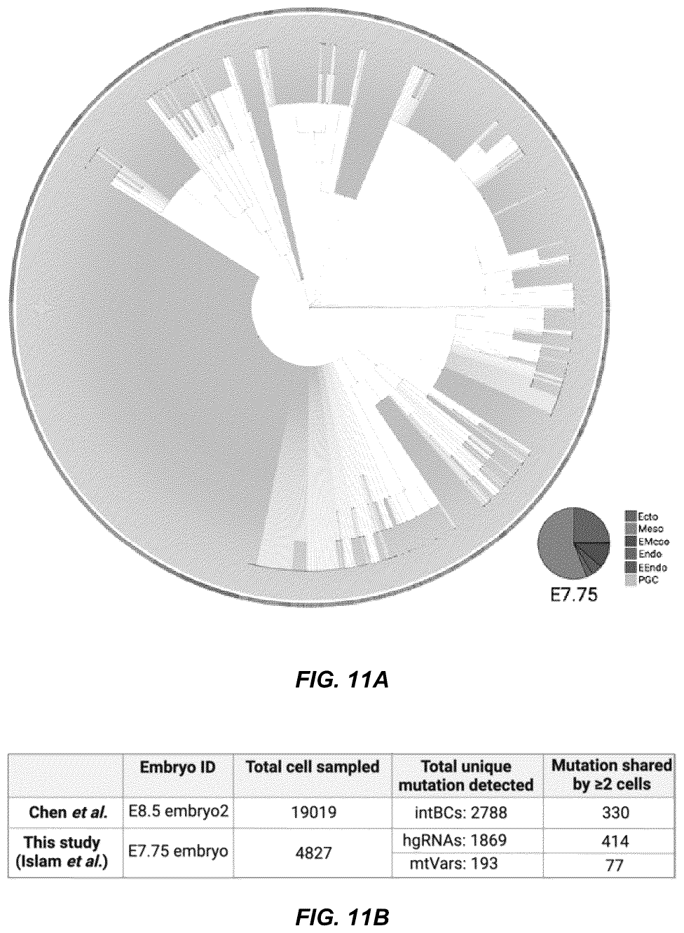

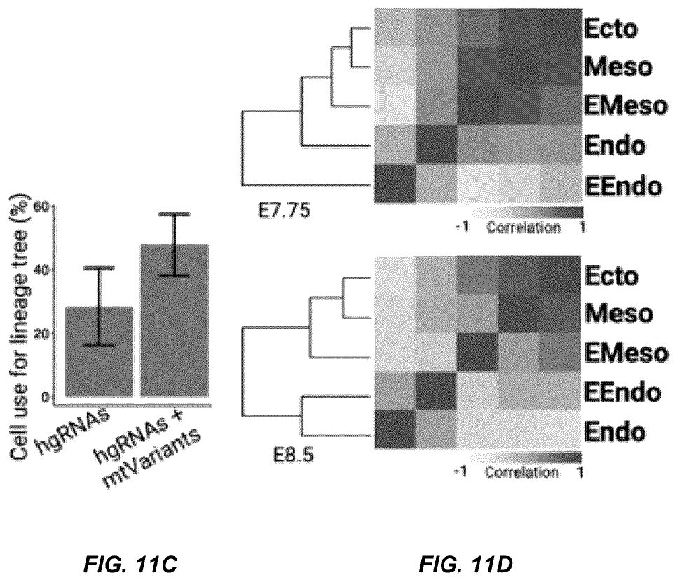

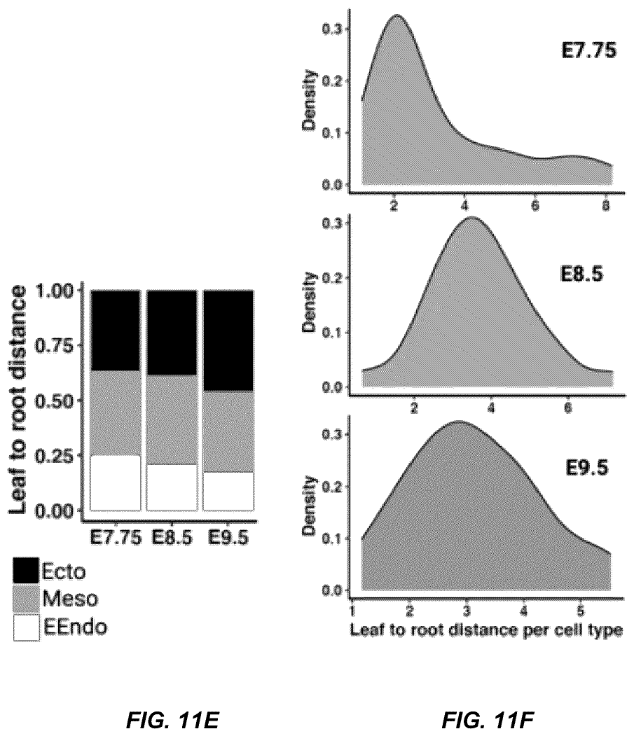

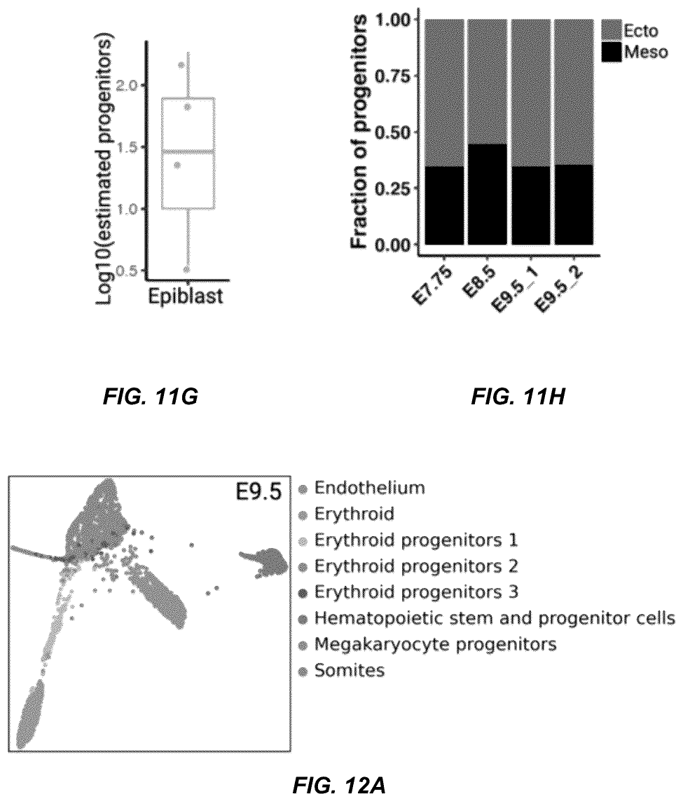

Phylogenetic distances (Abouheif [32]) were calculated for each leaf cell from non-binary single-cell lineage tree using distRoot function of adephylo package [Jombart, T., et al. Bioinformatics, 2010. 26(15):1907-9]. The distances were grouped by the respective germ layer or tissue types, followed by geometric mean calculation. Next, the relative distance for tissue types were converted to proportion (%) for each embryonic time point and plotted as density plots (FIGS. 11E-11F).

### Progenitor Population Size Estimation:

Progenitor population was calculated using previously reported phylogeny-based retrospective statistical inferences called targeting coalescent analysis (TCA) methods [He, X., et al., Research Square 2022]. Briefly, TCA used rooted phylogenetic tree as input to compute the average coalescent rate (CR) at the clades of the targeted tissues. The inverse of CR is a measure of the number of progenitors that are active in producing the descendant cell population. Epiblast progenitor population was calculated after assigning the germ layers (Ecto, Meso, Endo, EMeso) derived cells as epiblast descendent.

### Transcriptome Sequence Alignment and Quality Control:

Alignment was done using previously reported approach [Chen, B., et al., STAR Protoc, 2021. 2(2):100450]. Briefly, the DropEst pipeline [Petukhov, V., et al., Genome Biol, 2018. 19(1):78] use STAR aligner [Dobin, A., et al., Bioinformatics, 2013. 29(1):15-21] with the Ensembl reference genome to map reads to a genome, quantify transcript abundance and generate a cells X genes counts matrix after read alignment. The droplet raw matrix was processed as a scanpy AnnData object [Wolf, F. A., et al. Genome Biol, 2018. 19(1):15]. High-quality, cell-containing droplets were identified through finding the inflection point of the cumulative sum curve, and droplets with low information content were removed as reported before [Chen, B., et al., Cell, 2021. 184(26):6262-6280.e26]. The full protocol for running this QC pipeline is described by Chen et al. [Chen, B., et al., Cell, 2021. 184(26):6262-6280.e26]. Second, genes that were detected in fewer than 5 cells were filtered out. Potential doublets using scrublet [Wolock, S. L., et al. Cell Syst, 2019. 8(4):281-291.e9] were removed on a per sample basis with 10 principal components. Cells with high mitochondrial and ribosomal count percentages were also removed. Third, for each dataset, 4000 Highly Variable Genes (HVGs) were identified for downstream analysis using seurat v3 [Stuart, T., et al., Cell, 2019. 177(7):1888-1902.e21]. Fourth, the filtered count matrix was normalized to median library size. Normalized counts were log transformed for variance stabilization. Fifth, data was projected onto principal components with selected highly variable genes for dimension reduction. The number of components selected by the elbow method [Zhuang, H., et al. Bioinformatics, 2022. 38(10):2949-2951] were retained for each of the downstream analyses. Batch correction was done by using Harmony [Korsunsky, I., et al. Nat Methods, 2019. 16(12):1289-1296].

### Low-Dimensional Embedding:

A k-Nearest Neighbors (kNN) graph was constructed with 25 neighbors using Euclidean distance in principal components space as the inputs. A two-dimensional Uniform Manifold Approximation and Projection (UMAP) embedding was generated to increase signal-to-noise ratio. Force directed graphs were computed using the ForceAltas2 python module using the affinity matrix as input. Diffusion maps were generated with the Palantir pipeline [Setty, M., et al., Nat Biotechnol, 2019. 37(4):451-460]. The diffusion map is used for MAGIC imputation of missing data [van Dijk, D., et al., Cell, 2018. 174(3):716-729.e27]. For analysis with multiple time-points, an augmented affinity matrix generated by Harmony was used as the input for determining the diffusion components instead of the affinity matrix derived from the kNN graph alone as reported before [Nowotschin, S., et al., Nature, 2019. 569(7756):361-367].

### Unsupervised Clustering and Cell Type Labeling:

Single cell subclusters were labeled in UMAP through the Leiden algorithm, as part of the Scanpy toolkit. Gut cell type-specific marker gene list was curated form previous study [Banerjee, A., et al., Gastroenterology, 2020. 159(6):2101-2115.e5; Haber, A. L., et al., Nature, 2017. 551(7680):333-339]. Leiden clusters were assigned to specific cell types based on expression of marker genes. For late stage (E14.5) embryonic gut epithelium, midgut and hindgut tissues were enriched during embryo dissection. In addition, a fraction of yolk sac tissue was added to the tube before single cell dissociation. The major cell-states were assigned using scMRMA [Li, J., et al., Nucleic Acids Res, 2022. 50(2): e7]. The assigned epithelial cells were further re-clustered and annotated to respective cell types using marker genes from previous report [Fazilaty, H., et al., Cell Rep, 2021. 36(5):109484] and manual curation (FIG. 14H). Early embryonic cell type clustering and annotation was described in a separate section below.

### RNA Velocity and Pseudo-Time Ordering:

A separate spliced and unspliced count matrix for each sample was constructed from bam files using velocyto [La Manno, G., et al., Nature, 2018. 560(7719):494-498] command mapped to comprehensive gene annotation for CHR region. Spliced and unspliced counts were concatenated, filtered, and normalized using scVelo [Bergen, V., et al., at Biotechnol, 2020. 38(12):1408-1414]. RNA velocity vector fields were computed using the scVelo stochastic model of transcriptional dynamics. Velocities were then visualized on top of the same UMAP embeddings. Palantir [Setty, M., et al., Nat Biotechnol, 2019. 37(4):451-460] was used to model pseudo-time ordering for embryonic cell populations. Diffusion map-based low dimension embedding of the scRNA-seq data is an approximation of the phenotypic landscape occupied by the cells. Pseudo-time ordering of cells was calculated using a manually defined start cells and order was defined using shortest path distances in a kNN graph constructed in the phenotypic manifold. Force directed graph was constructed using diffusion maps as input and Palantir pseudotime was overlaid on the same embedding.

### Immunofluorescence, Whole-Mount, and Histological Imaging:

After euthanasia of mouse, small intestinal tissue was removed and washed with 1×DPBS. The tissue section was longitudinally spread onto Whatman filter paper to flay open and fixed in 4% PFA (Thermo Scientific) overnight. Fixed tissue was washed with 1×DPBS, swiss-rolled, and stored in 70% EtOH until processing and paraffin embedding at The Translational Pathology Shared Resource (TPSR) core in Vanderbilt University. Tissues were sectioned onto glass slides (5 μm) and processed using reported steps [Chen, B., et al., Cell, 2021. 184(26):6262-6280.e26], including deparaffinization, rehydration, and antigen retrieval. Overnight incubation was done for primary antibodies in a humidity chamber, followed by PBS wash (3×), compatible conjugated secondary antibody (1:500), and DAPI staining for 1 h. Slides were washed and mounted in Prolong Gold (Invitrogen) and imaged using Nikon A1R. For histological analysis, slides were processed and stained for hematoxylin and eosin. Whole-mount was done for isolated crypt using Tob2 antibody (Invitrogen, Catalog #PA5-62923).

### Fluorescence In Situ Hybridization:

Collected mouse embryos were fixed with 2% PFA in room temperature for 1 hour, followed by overnight sucrose (30%) incubation at 4° C. and OCT (Sakura, #4583) embedding. Embryos were sectioned onto glass slides (8 μm). Custom designed HCRFISH probes were purchased from Molecular Instruments, Inc. In situ HCRv3 approach [Choi, H. M. T., et al., Development, 2018. 145(12)] was applied to visualize markers using manufacturer protocols. Slides were soaked in DAPI solution at room temperature before mounting in Prolong Gold (Invitrogen). Images were generated using Nikon A1R.

### Organoid Culture:

Duodenal tissue from MARC1; Cas9+/− mouse was dissected, and crypts were enriched using reported EDTA-based approach as mentioned before. The final crypt pellet (˜10 μL) was resuspended in 300 μL of Matrigel and embedded in 12-well dish. The Matrigel was suspended in IntestiCult Organoid Growth Medium (Stem Cell Technologies, Vancouver, BC, Canada) supplemented with Primocin antimicrobial reagent (1:1000; InvivoGen, San Diego, CA). The organoids were dissociated and continuously passaged for 1 month using previously reported protocol [Sato, T., et al., Nature, 2009. 459(7244):262-5]. After every two weeks, the organoids were subsampled for DNA extraction and barcode sequencing.

### Human Sporadic Polyps Sample Collection:

Human tissue samples were collected using previously reported approach [Chen, B., et al., Cell, 2021. 184(26):6262-6280.e26]. Briefly, blood was collected using IV line to EDTA and serum tube, prior to colonoscopy, and spin down at 1500 g for 10 minutes to collect white blood cells, followed by DNA extraction using QIAmp DNA kit (QIAGEN). Polyp tissue was collected using colonoscopy per standard clinical practice in the Vanderbilt Pathology Laboratory and immediately placed into RPMI media before scRNA-seq experiment. A portion of the polyps were placed into formalin for fixing and diagnosis using standard clinical practice. For whole exome sequencing of polyps, DNA was purified with the truXTRAC FFPE microTUBE DNA Kit-Column Purification kit (Covaris). Tissue was scraped from a few 10 μm FFPE sections, deparaffinized using xylene, and lysed in an optimized lysis buffer that contains proteinase K. Following the proteinase K digestion to release DNA from the tissue, a higher temperature was used incubation to reverse formalin crosslinking alongside RNase treatment using RNase A (Thermo Fisher). The DNA samples were stored at −80° C. before being used for assays.

### Human Single-Cell Encapsulation, Library Generation and Alignment:

Colonic biopsy from RPMI solution was minced to approximately 4 mm², and washed with 1×DPBS and incubated in chelation buffer (4 mM EDTA, 0.5 mM DTT) at 4° C. for 1 h 15 min. Next, the tissue was dissociated into single cell using cold protease and DNase I for 25 minutes as reported before [Banerjee, A., et al., Gastroenterology, 2020. 159(6):2101-2115.e5; Chen, B., et al., Cell, 2021. 184(26):6262-6280.e26; Simmons, A. J., et al., STAR Protoc, 2022. 3(3):101570]. Cells were encapsulated using a modified inDrop platform [Klein, A. M., et al., Cell, 2015. 161(5):1187-1201], and sequencing libraries were prepared using the TruDrop protocol [Southard-Smith, A. N., et al., BMC Genomics, 2020. 21(1):456]. Illumina NovaSeq 6000 was used to sequence the library in a S4 flow cell using a PE150 kit to a target of 150 million reads. DropEst pipeline [Petukhov, V., et al., Genome Biol, 2018. 19(1):78] was used for demultiplexing aligning and correcting the detected read counts of a library.

### Whole Exome Sequencing and Data Analysis:

Human WES was performed on S4 flow cells on NovaSeq6000 (PE150) to the targeted coverage. The reads were aligned to the human reference genome hg19 using BWA [Li, H. et al. Bioinformatics, 2009. 25(14):1754-60] and indexed by Sambamba [Tarasov, A., et al., Bioinformatics, 2015. 31(12):2032-4]. Duplicated reads were removed using Picard. Somatic mutations were called using ‘normal-tumor’ paired mode in GATK4 Mutect2 [Van der Auwera, G. A., et al., Curr Protoc Bioinformatics, 2013. 43(1110):11]. Variants were annotated using ANNOVAR [Wang, K., et al. Nucleic Acids Res, 2010. 38(16): e164]. For mouse WES, DNA was extracted from resected tumors and normal epithelial tissues using DNA Extraction Kits (Qiagen). Standard whole exome sequencing (WES) library was prepared using mouse exome panel (Twist Bioscience) and sequenced on S4 flow cells on NovaSeq6000 (PE150) to the targeted coverage (50×). WES reads were aligned to the mouse reference genome (mm 10) using BWA [Li, H. et al. Bioinformatics, 2009. 25(14):1754-60] and indexed by Sambamba [Tarasov, A., et al., Bioinformatics, 2015. 31(12):2032-4]. Somatic variants were called using GATK Mutect2 [Van der Auwera, G. A., et al., Curr Protoc Bioinformatics, 2013. 43(1110):11]. Variants were annotated using ANNOVAR [Mayakonda, A., et al., Genome Res, 2018. 28(11):1747-1756] and germline variants were filtered using paired normal samples. Variants were further analyzed and visualized using Maftools [Mayakonda, A., et al., Genome Res, 2018. 28(11):1747-1756].

### TCPS Targeted DNA Sequencing, Alignment and Mutation Calling:

Tennessee colorectal polyp study (TCPS) sample collection and DNA extraction was reported before [Chen, B., et al., Cell, 2021. 184(26):6262-6280.e26]. Briefly, DNA was extracted from FFPE section using same approach as DNA extraction for WES. A curated gene panel was used for targeted DNA sequencing, including APC, and read depth was 500×. All primer development and next-generation sequencing were conducted by Covance. Alignment and mutation calling were done using similar approach as WES. See Chen et al. for further details [Chen, B., et al., Cell, 2021. 184(26):6262-6280.e26]. Number of deactivating APC mutations was quantified per polyps and analyzed using R and visualized using complex heatmap package.

Mutation Calling from scRNA-Seq Reads:

We used two independent methods (SComatic [Muyas, F., et al., Nature Biotechnology, 2023] and Monopogen [Dou, J., et al., Nat Biotechnol, 2023]) to call mutations from human scRNA-seq reads as per protocol. Briefly, we used abnormal cells including adenoma specific cells (ASC) and sessile serrated cells (SSC) as two major abnormal cell types in polyps identified in our previous HTAN study [Chen, B., et al., Cell, 2021. 184(26):6262-6280.e26]. We also use polyps infiltrating immune cells (IMM) as reference and called mutation from as pseudobulk using SComatic. Similar mutations were detection between SComatic and Monopogen. Due to less run time required and reliable mutation calling from chrX, SComatic was used as main mutation calling approach for scRNA-seq reads.

### Medina VAF and X Chromosome Inactivation Mosaicism:

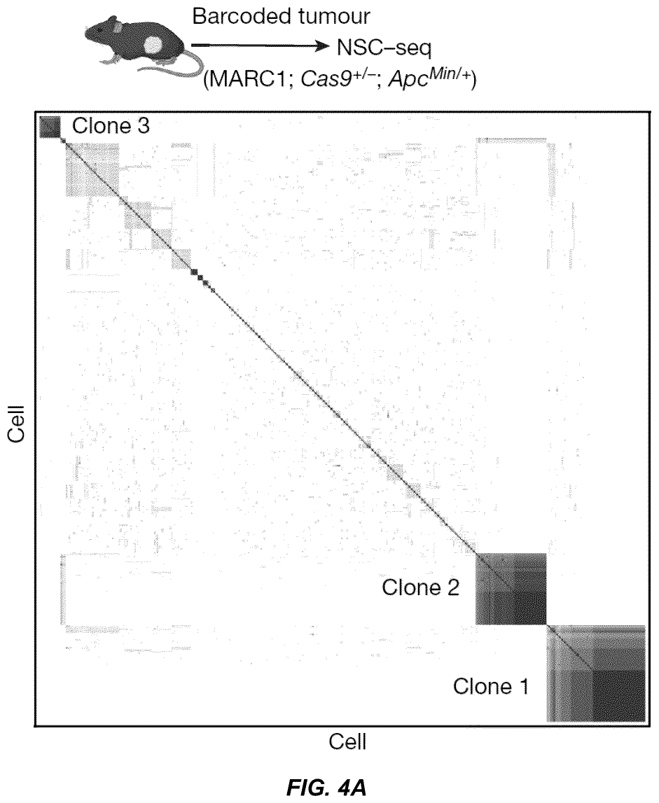

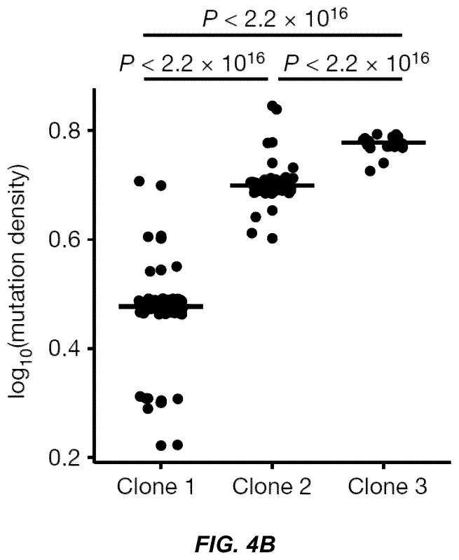

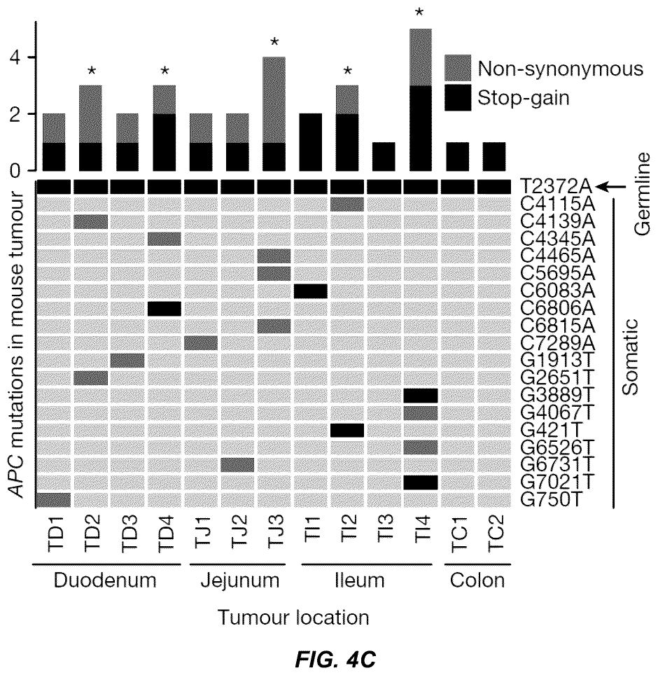

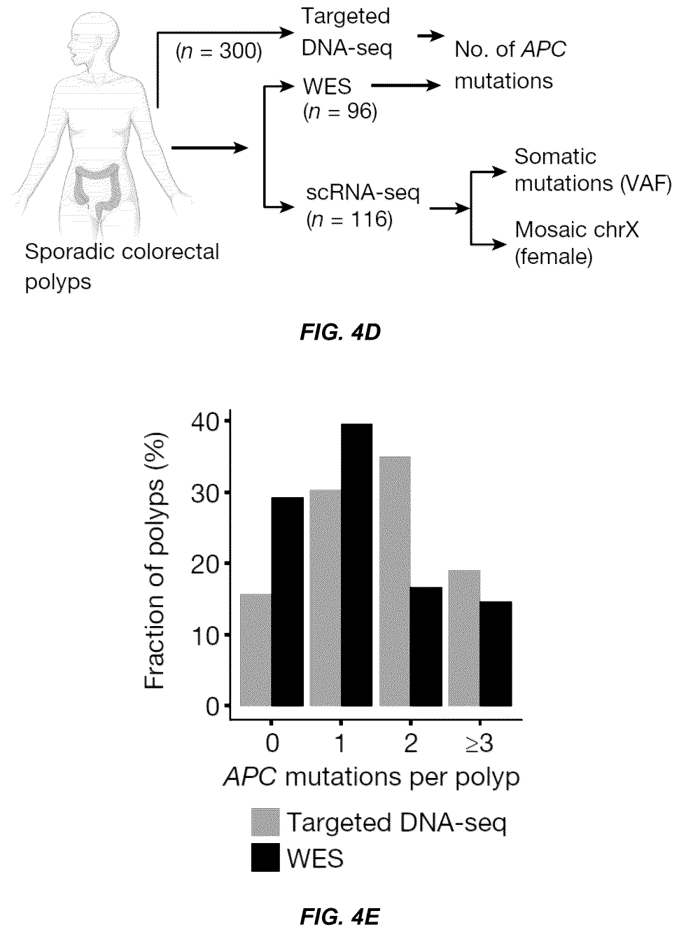

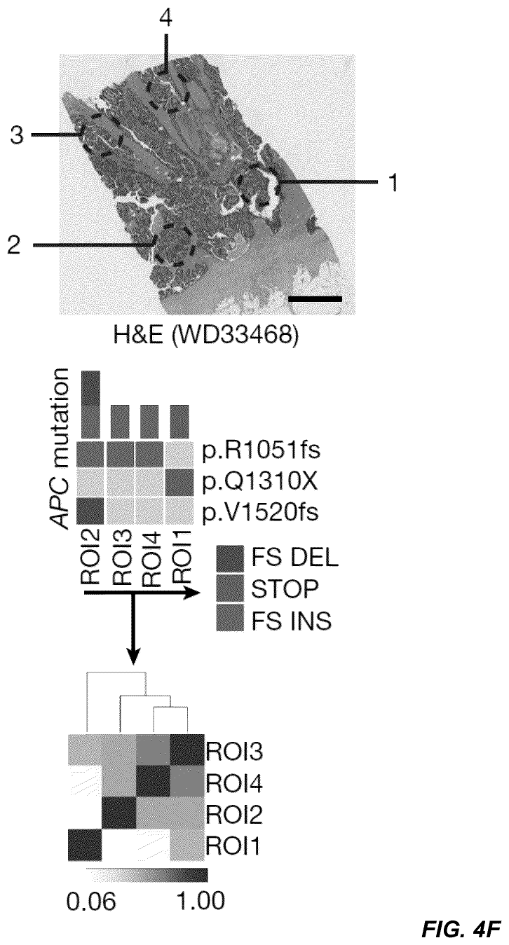

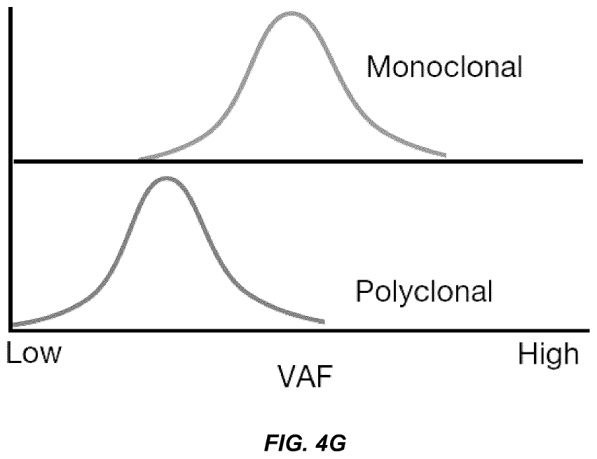

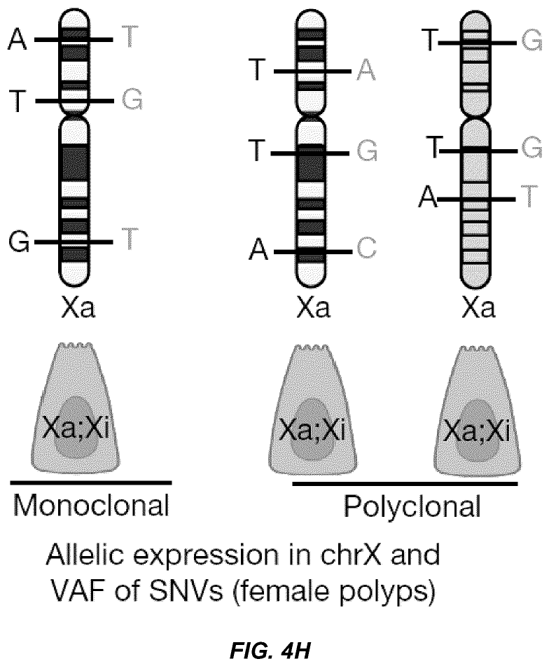

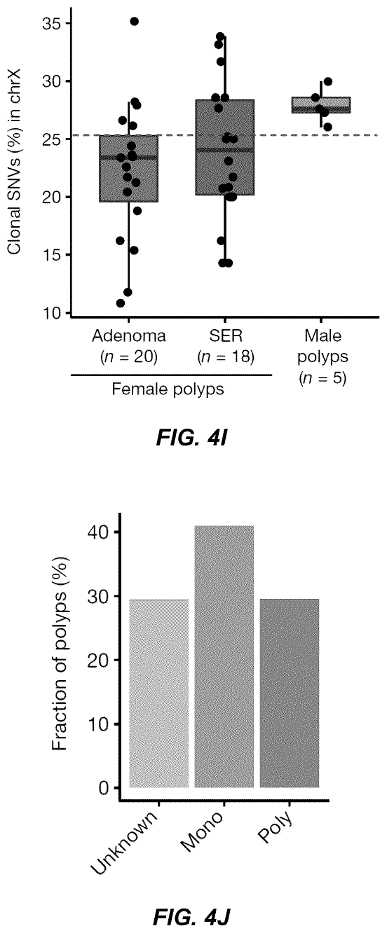

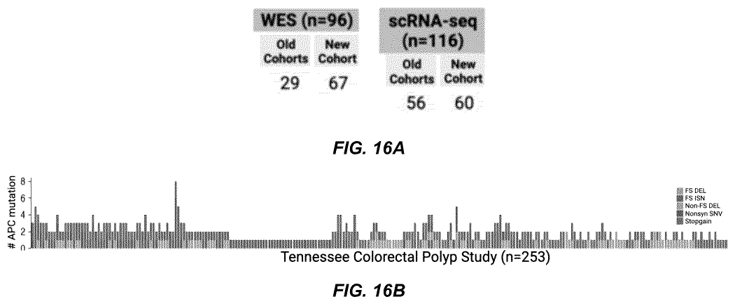

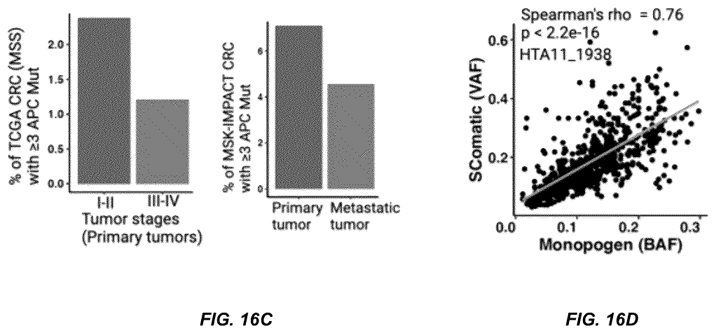

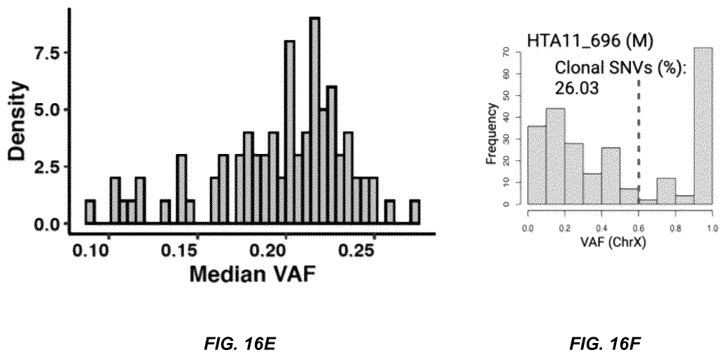

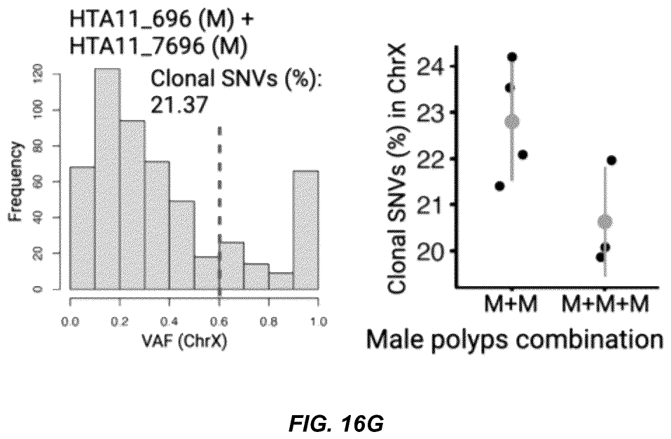

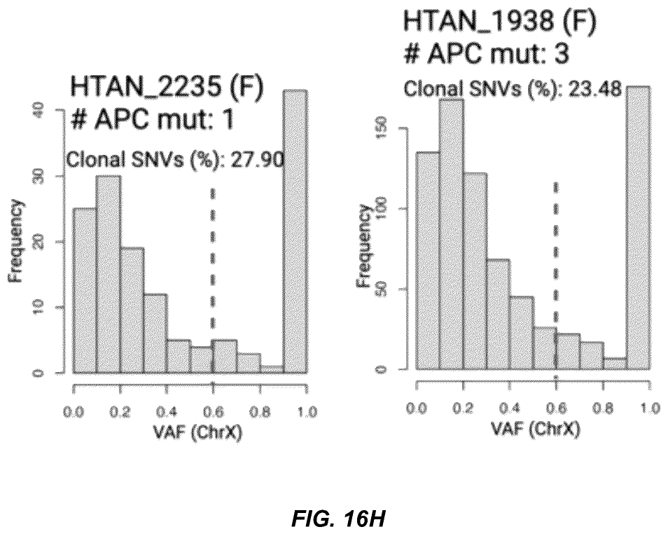

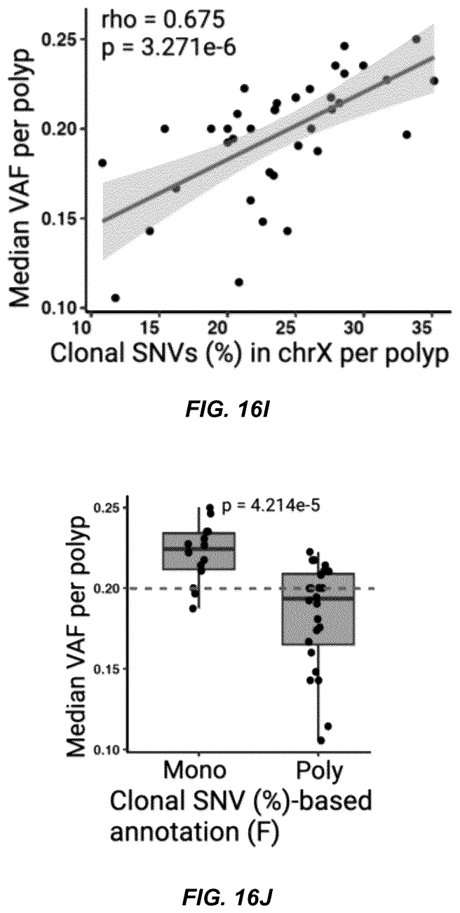

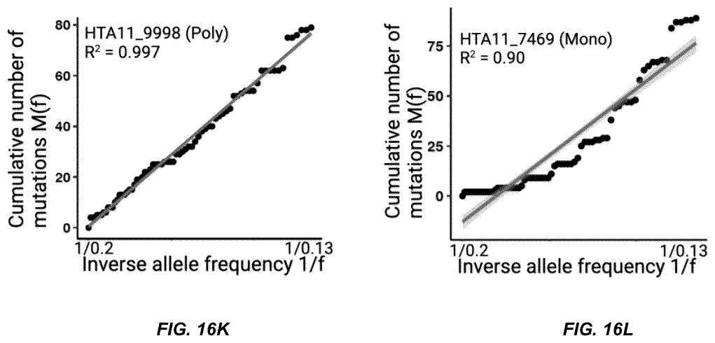

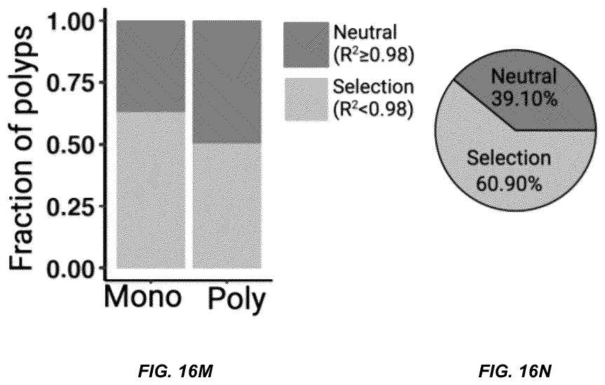

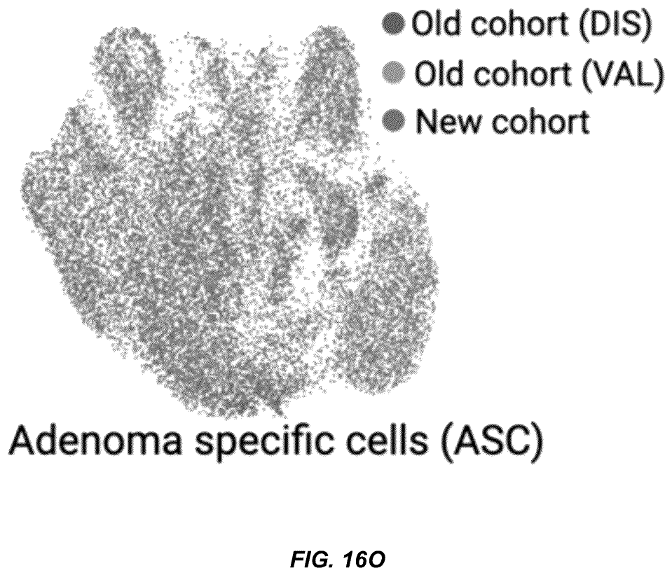

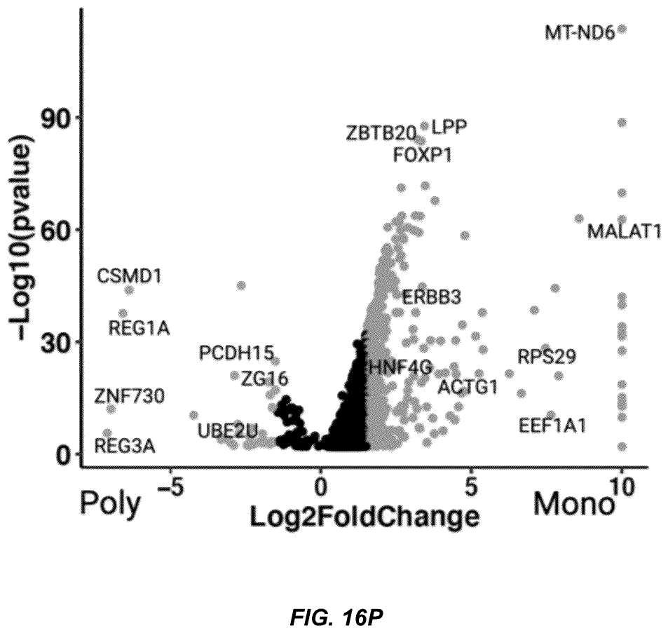

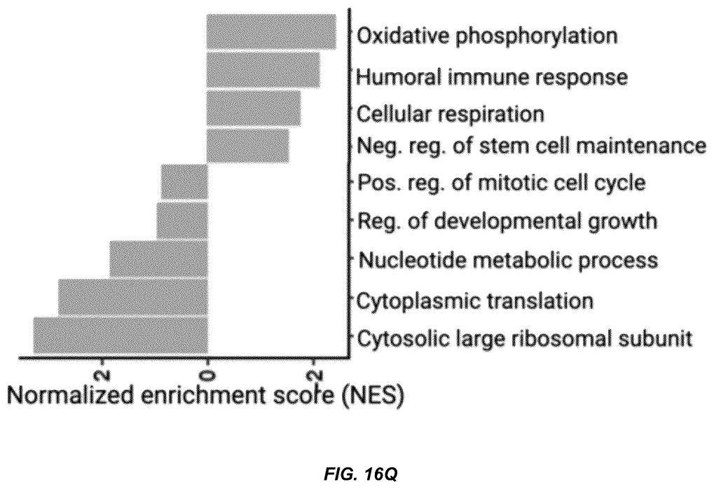

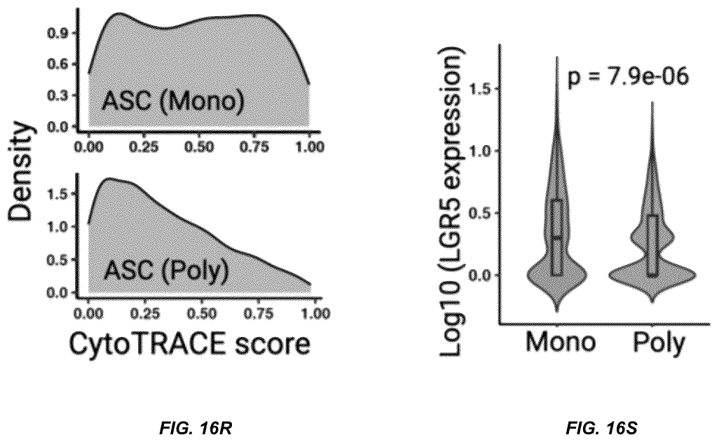

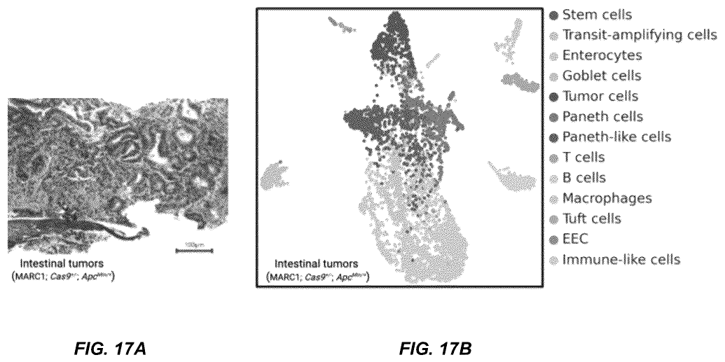

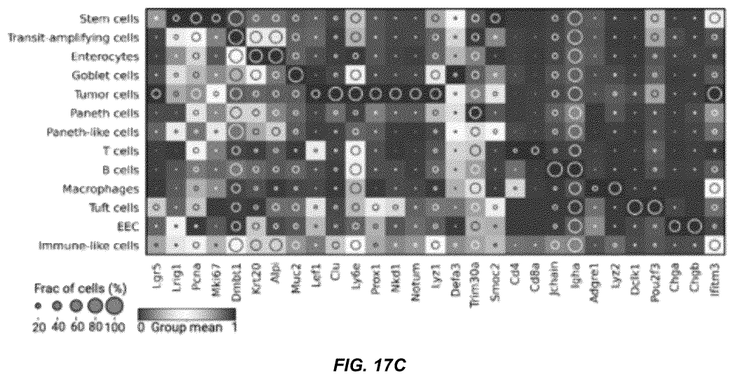

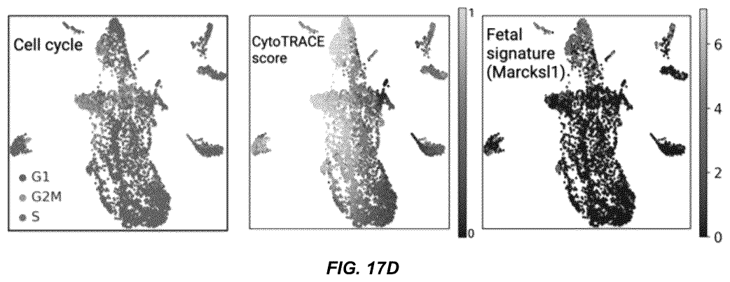

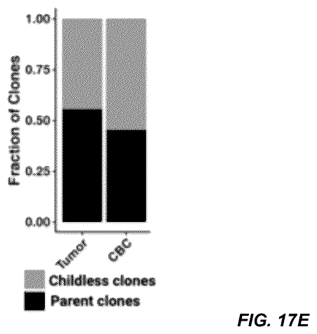

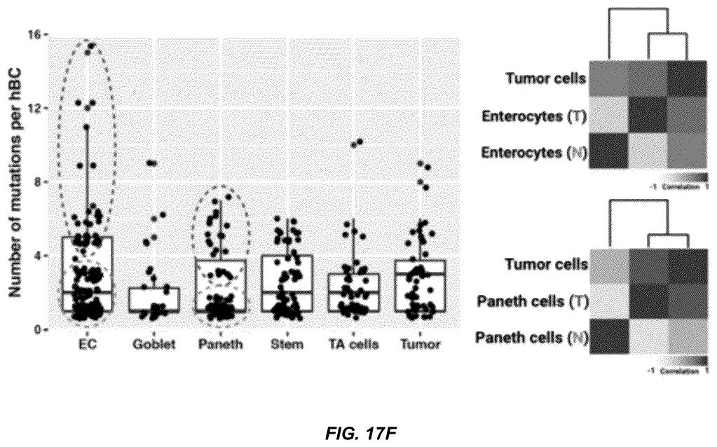

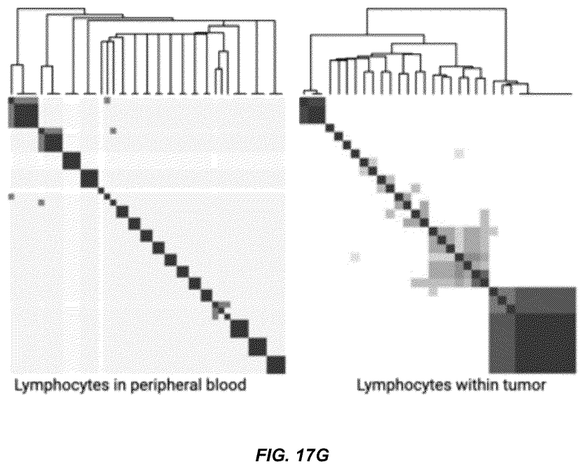

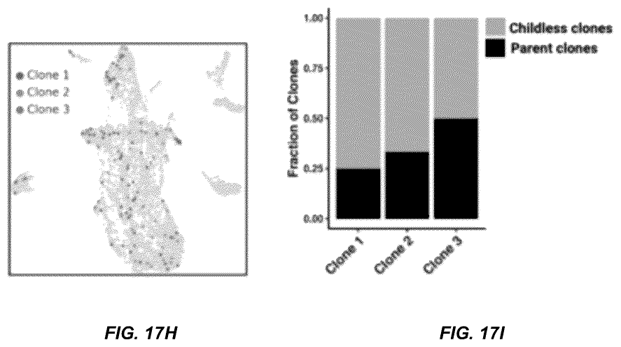

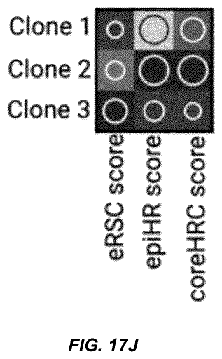

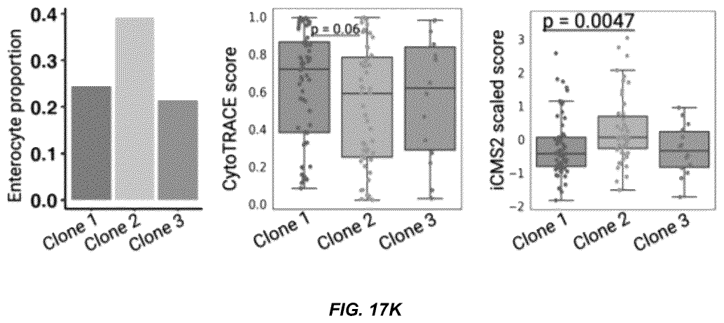

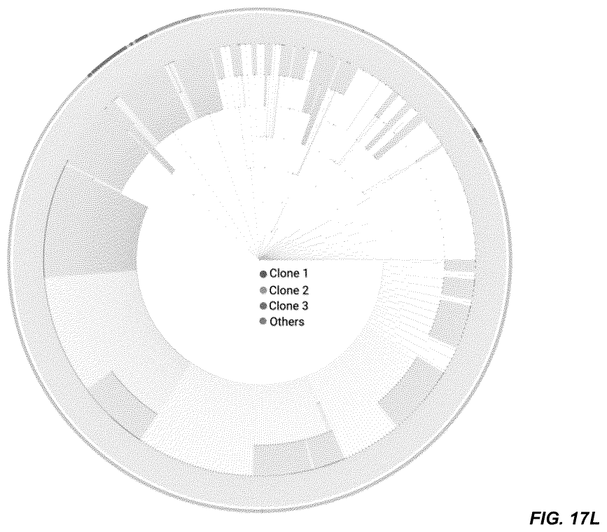

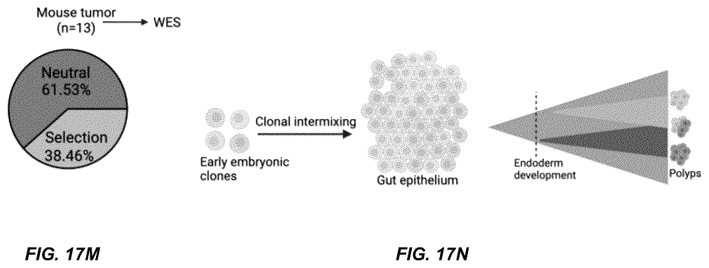

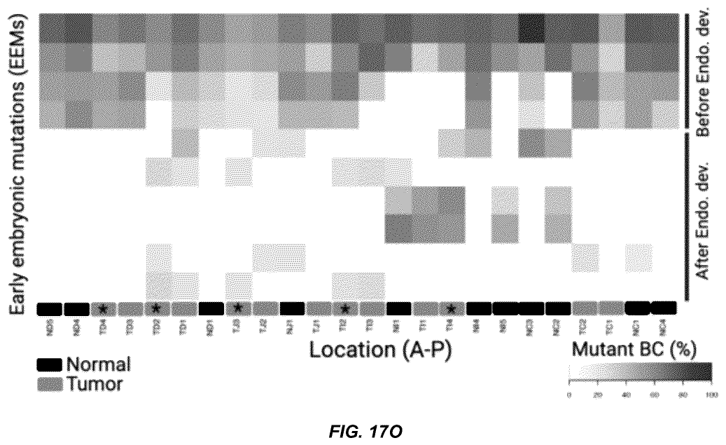

To investigate mosaic pattern of chrX inactivation in human polyps, we analyzed HTAN polyps (adenoma) scRNA-seq data from both male and female samples. SComatic was used to call SNVs from abnormal cells (ASC/SSC) and polyps infiltrating immune cells (IMM) as pseudo-bulk. SNVs from IMM cells were used to filter our germline variants from abnormal cells. Next, we only use the SNVs that pass cell type filter and total read depth >10. In addition, SNVs were removed if VAF>0.6 and VAF<0.04 as possible germline and noise, respectively. We also filtered out SNVs from sex chromosome (X or Y) from both male and female samples for median VAF calculation. Finally, we quantified median VAF per samples using dplyr package (FIG. 16E). For X-linked somatic SNVs, we only used SNVs from X chromosome that pass cell type specific filter and read depth ≥10 and similar germline filter using IMM cells. Furthermore, we removed the X-linked SNVs that have been associated to genes reported as escape, express from inactive chrX [Tukiainen, T., et al., Nature, 2017. 550(7675):244-248; Garieri, M., et al., Proc Natl Acad Sci USA, 2018. 115(51):13015-13020]. Finally, VAF>0.6 is considered as clonal SNVs for each polyp and plotted as box plot for both male and female polyps (FIG. 4G).

### Selective and Neutral Evolution Calculation:

Tumor evolutionary processes were calculated using reported approach [Williams, M. J., et al. Nat Genet, 2016. 48(3):238-244] from WES data. Briefly, after filtering germline and low-quality mutations from human WES, we carefully selected subclonal SNVs (nonsynonymous) using VAF cut-off fmin=0.13 and fmax=0.2. For mouse WES data, we used a read depth (>25) and subclonal (0.13 to 0.24) cut-off. Then, R² value was calculated using reported approach in R package “neutralitytestr” that fit a linear model for calculating tumor evolution process [Caravagna, G., et al., Nat Genet, 2020. 52(9):898-907]. Polyps are assigned into selective (R²<0.98) or neutral (R²>0.98) evolution category based on R² values [Williams, M. J., et al. Nat Genet, 2016. 48(3):238-244].

### Results

### A Temporal Recording Pipeline:

To enable CRISPR-based temporal recording at single-cell resolution, one must address challenges of 1) non-polyadenylated hgRNA (but also sgRNA, stgRNA) capture, and 2) single cell data sparsity. We developed custom capture of non-polyadenylated gRNA that does not require redesign of whole gRNA libraries [Replogle, J. M., et al., Nat Biotechnol, 2020. 38(8):954-961] or indirect readouts [Dixit, A., et al., Cell, 2016. 167(7):1853-1866.e17; Adamson, B., et al., Cell, 2016. 167(7):1867-1882.e21; Datlinger, P., et al., Methods, 2017. 14(3):297-301] (FIGS. 1A, 5A). ˜80% of gDNA mutations were detected in hgRNA with NSC-seq (FIG. 1B). We demonstrated hgRNA mutations were equivalent to gDNA ones for lineage tree reconstruction using controlled cell passage experiments (FIG. 5B). Adaptation of NSC-seq to single-cell resolution using inDrops [Klein, A. M., et al., Cell, 2015. 161(5):1187-1201] demonstrated gRNA detection in 95% of identified single cells in cell line experiments, with their paired transcriptome data exhibiting similar quality as regular inDrops (FIGS. 1C-1D, 5C-5F). The effectiveness of using single-cell data is diminished by data sparsity, as illustrated by the applications to track cell division history and construct lineage trees. Previous work [Park, J., et al., Cell, 2021. 184(4):1047-1063.e23] and our results here show that gDNA barcode mutation frequency—as defined by ratio of mutated vs. wildtype barcodes-tracks linearly with cell or organoid culture time when measured in bulk (FIG. 6A-6C). Due to single-cell data sparsity, only a fraction of barcodes can be detected on a per cell basis, invalidating the mutational frequency metric. We found that mutation density—as defined by average number of mutations in barcodes—is immune to data sparsity and also tracks with time in organoid cultures and the renewing intestinal epithelium (FIG. 6D-6E). We revealed that mutational density increases at a faster rate in intestinal organoid cultures than the in vivo intestinal epithelium (FIG. 6F), confirming that epithelial cells in organoid conditions are more proliferative [Huelsz-Prince, G., et al., Elife, 2022. 11]. Cellular turnover rates of common intestinal cell types, as inferred by mutational density, were also consistent with current knowledge (FIG. 6G). Specifically, tuft cells exhibited a multimodal distribution of mutational densities, consistent with a heterogeneous population with different lifetimes [Banerjee, A., et al., Gastroenterology, 2020. 159(6):2101-2115.e5; Westphalen, C. B., et al., J Clin Invest, 2014. 124(3):1283-95] (FIG. 6H). NSC-seq applied to three murine embryonic time points to profile hgRNAs and mRNAs simultaneously also showed mutation density to increase over time (FIG. 1E-1F), driven by cell type-specific changes (FIG. 6I-6J), but with no cell type bias in Cas9 expression or NHEJ activity (FIG. 6K-6L). While mutation density per barcode can be used for timing assessments, non-overlapping gRNA barcode expression detected per cell also limits information content used for cell phylogeny reconstruction. We thus augmented hgRNA mutational information with somatic mitochondrial variants (mtVars). Briefly, we filtered out germline mtVars using a custom ‘germline mtVars bank’, and then defined a lineage determining cutoff from mtVar distributions using paired hgRNA mutations as ‘ground truth’ somatic variants (FIG. 7A-7D). Using this pipeline, we showed that mtVars also consistently increased over three embryonic time points (FIG. 1G-1H), similar to hgRNA mutations (FIG. 1F). We further delineated the known developmental order of different murine brain layers prior to left/right brain segregation [Kiecker, C. et al., Annu Rev Neurosci, 2012. 35:347-67], and verified previously reported clonal relationships between 3 human breast cancer regions, solely using mtVars on published spatial data [Vickovic, S., et al., Nat Methods, 2019. 16(10):987-990; Wei, R., et al., Nat Biotechnol, 2022. 40(8):1190-1199]. Single-cell analysis using hgRNA, mtVars, or both were able to accurately distinguish lymphoid and myeloid cells as distinct lineages in PBMCs and differentiate embryonic tissue types (FIG. 1I). Together, we developed a comprehensive pipeline of temporal and lineage tracking that is compatible with paired single-cell transcriptomic analysis (FIG. 1J).

### Embryonic Lineage and Cell Division Tracking:

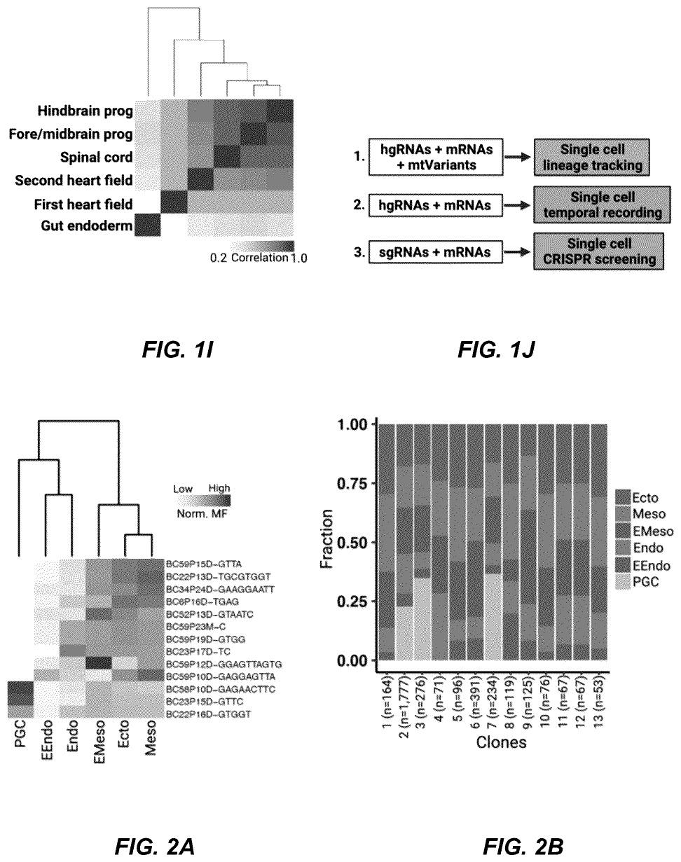

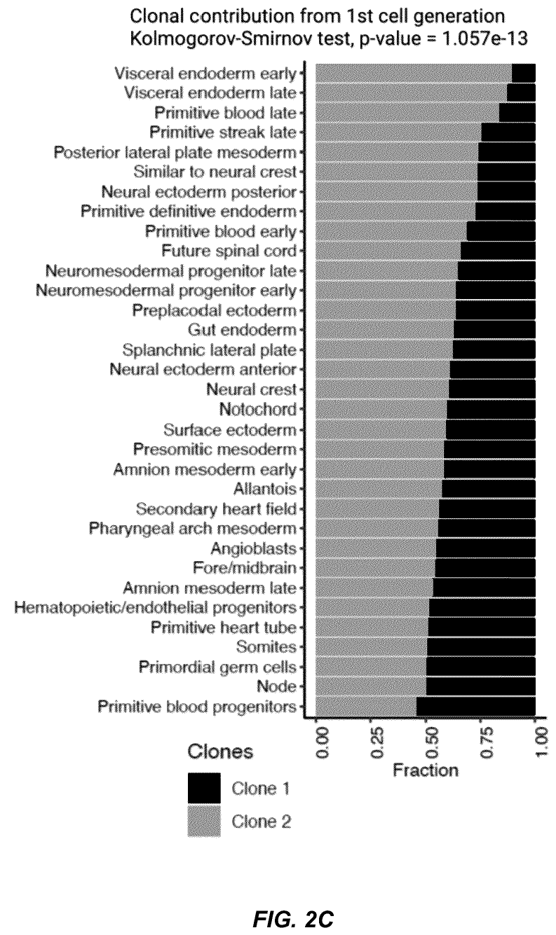

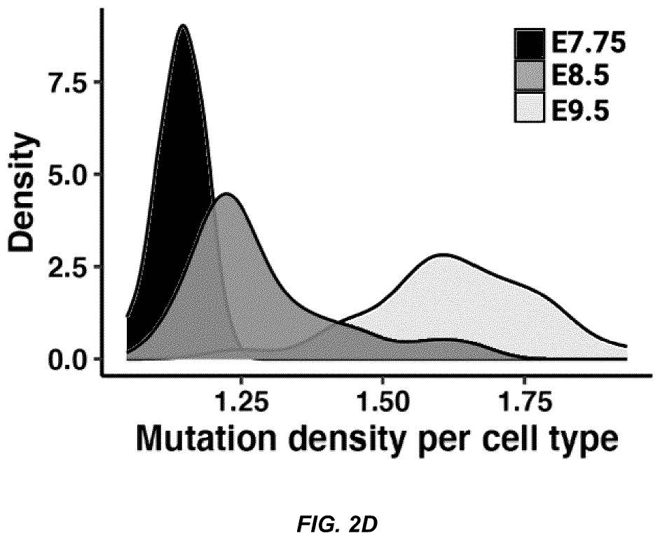

We then more deeply analyzed combined single-cell barcoding and transcriptome data of E7.75, E8.5, and E9.5 embryos to elicit biological insights from NSC-seq. Cell type annotation using conventional gene expression analysis revealed canonical cell types and germ layers in each of the time points [Chan, M. M., et al., Nature, 2019. 570(7759):77-82; Pijuan-Sala, B., et al., Nature, 2019. 566(7745):490-495; Imaz-Rosshandler, I., et al., bioRxiv, 2023:2023.03. 17.532833]. Notably, more defined cell types emerged at E9.5 compared to earlier time points (E7.75/8.5), consistent with the established timeline of mammalian development. This distinction prompted two separate sets of cellular annotations. Matching our data with previously generated scRNA-seq data at E7.0 and E8.0 supported the correct development timing of our single-cell embryonic data (FIG. 8), and data quality was typical of this experimental platform. Regarding the quality of barcode mutations called, the distribution of mutations amongst cells, the frequency of different types of mutations, the incidence of random collision mutations, the number of mutations as a function of cell type, and the barcode lengths were consistent with previous reports (FIG. 9A-9E) [Perli, S. D., et al. Science, 2016. 353(6304)]. Early embryonic mutations (EEM) occur during the initial stages of cell division in development and are inherited by a significant portion of cells within the embryo (FIG. 9F-9G). The proportional presence of these mutations among cells, referred to as the mosaic fraction (MF), can serve as an indicator of the cell generation (CG) where these mutations originated (FIG. 8) [Samuels, M. E. et al., Genes (Basel), 2015. 6(2):216-37]. Progressive restriction of EEMs shared in tissues enable the use of MFs to model early divergence of germ layers and tissue types (FIG. 6A). Mouse primordial germ cell (PGC) lineage segregated from other embryonic and extra-embryonic lineages, supporting possible early allocation of cells to the PGC lineage that has also been reported in mouse [Saitou, M. et al. Cold Spring Harb Perspect Biol, 2012. 4(11)] and human [Kobayashi, T., et al. Development, 2018. 145(16); Coorens, T. H. H., et al., Nature, 2021. 597(7876):387-392]. We also found a similar MF between mesoderm and ectoderm that supported a shared progenitor population, as reported before [Tzouanacou, E., et al., Dev Cell, 2009. 17(3):365-76]. Notably, extra-embryonic endoderm (EEndo) and embryonic endoderm (Endo) appeared to share origins, although they are known to originate from two distinct tissue layers, hypoblast and epiblast, respectively. However, there is literature support for some degree of shared progenitors, lineage convergence, and intermixing between these tissues [Chan, M. M., et al., Nature, 2019. 570(7759):77-82; Pijuan-Sala, B., et al., Nature, 2019. 566(7745):490-495; Nowotschin, S., et al., Nature, 2019. 569(7756):361-367; Ibarra-Soria, X., et al., Nat Cell Biol, 2018. 20(2):127-134; Peng, G., et al., Nature, 2019. 572(7770):528-532; Kwon, G. S., et al., Dev Cell, 2008. 15(4):509-20]. We also assessed the clonal contributions of different EEMs towards germ layers (early) or tissue types (late), and found unequal contribution between different early clones (FIGS. 6B, 9J-9K). We observed unequal partition of first cell generation clones across different tissue types (FIG. 2C, p=1.057-13), suggesting that the specific lineage commitment of early embryonic progenitors is not predetermined, but rather subject to potential induction or stochastic processes (FIG. 9L-9M). This phenomenon has been previously reported in mammals [Visvader, J. E., Nature, 2011. 469(7330):314-22; Hoadley, K. A., et al., Cell, 2018. 173(2):291-304.e6; Dixit, A., et al., Cell, 2016. 167(7):1853-1866.e17; Li, Z., et al., Cell Stem Cell, 2012. 11(5):663-75; Qiu, J., et al., J Mol Cell Biol, 2016. 8(4):288-301], in contrast to the C. elegans model.

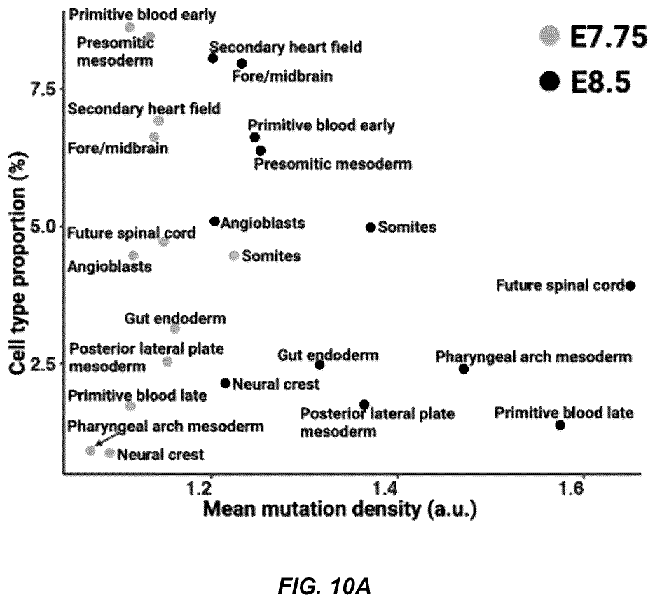

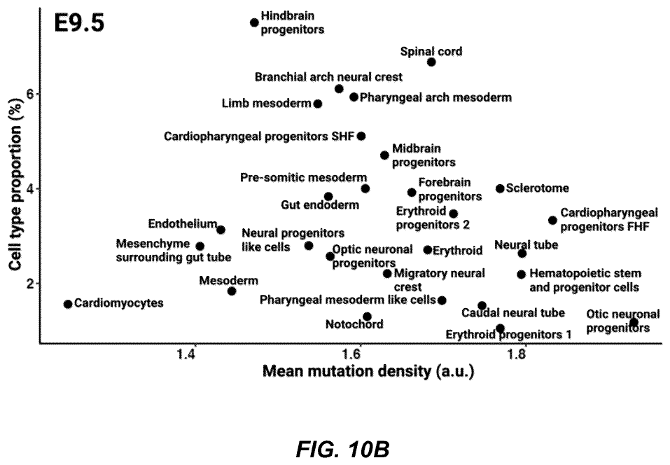

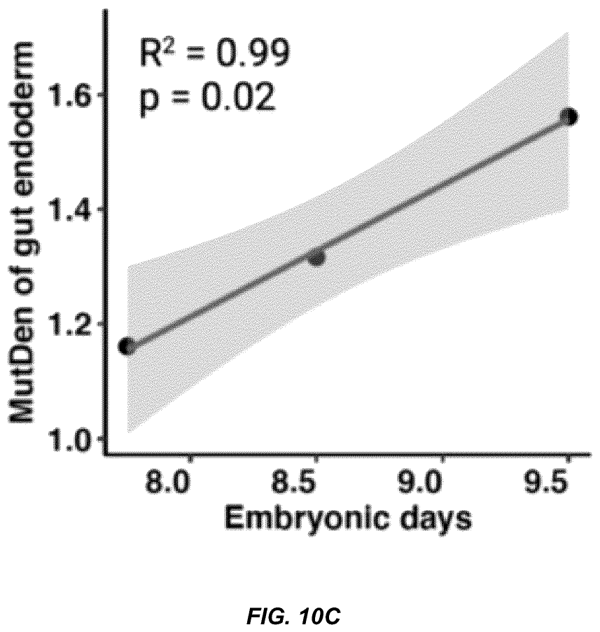

The regulation of organ sizes is one of the most fundamental processes of embryonic development, primarily governed by organ-specific cell division rates, and to a lesser extent, apoptosis rates [Bryant, P. J. et al., Q Rev Biol, 1984. 59(4):387-415; Stanger, B. Z., Cell Cycle, 2008. 7(3):318-24; Conlon, I., et al. Cell, 1999. 96(2):235-44; Leevers, S. J., et al. Curr Opin Cell Biol, 2005. 17(6):604-9]. Here, we developed a catalog of cell division history of different organs to reveal insights into the timing and scale of cell division across tissues during development. Using mutations within NSC-seq barcodes, we quantified the cumulative number of cell divisions per tissue type at three gastrulation time points (FIG. 10A-10B). We observed that the relationship between the number of cell divisions and the known tissue mass differs among various tissue types, which could be attributed to differential progenitor field size, the timing of progenitor specification, cell death, cellular lifespan, and cell competition across tissue types [van Neerven, S. M. et al., Nat Rev Mol Cell Biol, 2023. 24(3):221-236]. Additionally, our data revealed a widening distribution of tissue-specific cumulative cell division levels at both E8.5 and E9.5 stages whereas a narrow unimodal distribution was observed for the E7.75 stage (FIG. 2D), suggesting that tissue-specific cell division and diversification initiates after the E7.75 stage. In general, we observed high proliferation of hematopoietic progenitors during gastrulation period, while cardiomyocytes and endothelium showed low proliferation (FIG. 10A-10B). We noticed an emergence of various intermediate hematopoietic progenitors at E9.5 with distinct cellular turnover histories, supporting diverse roots of hematopoiesis during early embryonic development as reported before [Yzaguirre, A. D., et al. Dev Dyn, 2016. 245(10):1011-28; Li, Z., et al., Cell Stem Cell, 2012. 11(5):663-75; Qiu, J., et al., J Mol Cell Biol, 2016. 8(4):288-301; Patel, S. H., et al., Nature, 2022. 606(7915):747-753; Notta, F., et al., Science, 2016. 351(6269): aab2116; Yokomizo, T., et al., Nature, 2022. 609(7928):779-784; Nguyen, P. D., et al., Nature, 2014. 512(7514):314-8]. Cumulative cell division levels for forebrain progenitors were higher than hindbrain progenitors (FIG. 10B), supporting known turnover kinetics to maintain relative brain region sizes during mammalian neurogenesis van Neerven, S. M. et al., Nat Rev Mol Cell Biol, 2023. 24(3):221-236; Nowakowski, R. S., et al., Results Probl Cell Differ, 2002. 39:1-25; Takahashi, T., et al., Dev Neurosci, 1997. 19(1):17-22; Barres, B. A., et al., Cell, 1992. 70(1):31-46]. In addition, we found a constant rate of cell proliferation for gut endoderm over embryonic time points, a rate similar to adult intestinal epithelial turnover (FIGS. 10C, 10E). Overall, differential proliferation timing and kinetics between organs during gastrulation were observed. These variations occasionally corresponded to the organ size, but not always. We also demonstrated that for certain tissues, proliferation rates were set during gastrulation and persisted throughout life [Li, Z., et al., Cell Stem Cell, 2012. 11(5):663-75]. Overall, this catalog serves as a basis for studying embryonic cellular proliferation kinetics and adds a temporal axis in lineage diversification [Tanay, A., et al. Nature, 2017. 541(7637):331-338] to complement lineage tracking.

Next, a single-cell phylogenetic reconstruction was conducted using NSC-seq data at a higher information content per cell than previous approaches (FIG. 7A-7C). Pseudo-bulk reconstruction of embryonic tissue relationship generally recapitulated canonical knowledge of germ layer development (FIG. 11D). Phylogenetic distance analysis from single cell tree supports the closer proximity of EEndo to root compared to Endo or Meso to root (FIG. 11E). A wider distribution of the phylogenetic distance across cell type was observed at E8.5 and E9.5 compared to E7.75 (FIG. 11F), supporting the initiation of tissue type diversification after E7.75 illustrated above (FIG. 2D) [Bardot, E. S., et al., Mech Dev, 2020. 163:103617]. Furthermore, computational inference from single-cell lineage tree topology estimated the number of epiblast progenitors (n=˜28) and extrapolated unequal progenitor field size between ectoderm and mesoderm stemming from these progenitors (FIG. 11G-11H) [Lawson, K. A., et al., Development, 1991. 113(3):891-911].

### Instances of Unconventional Embryonic Lineage Diversification:

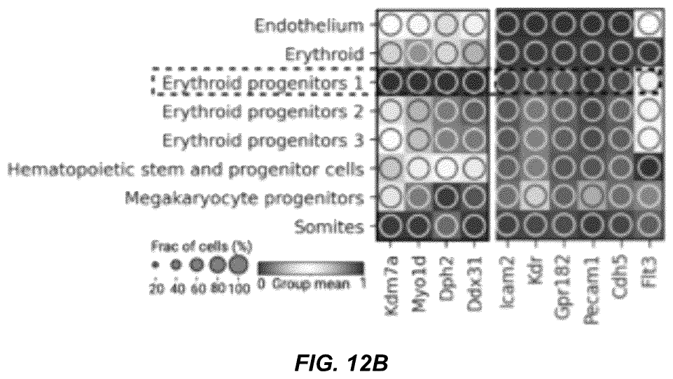

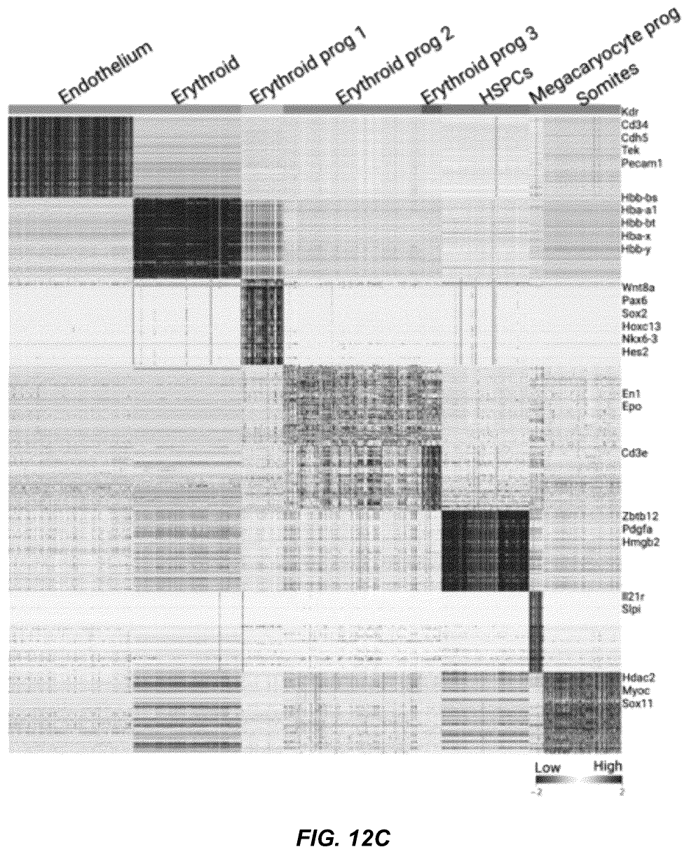

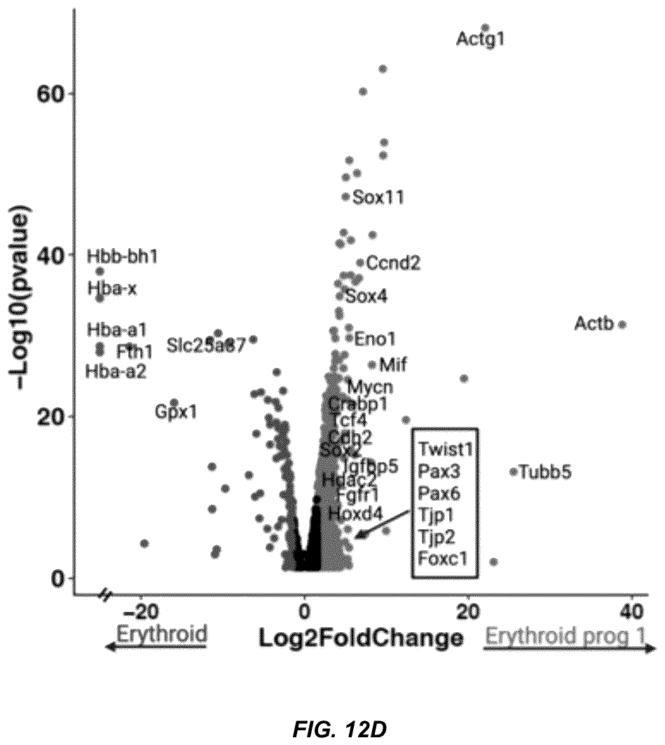

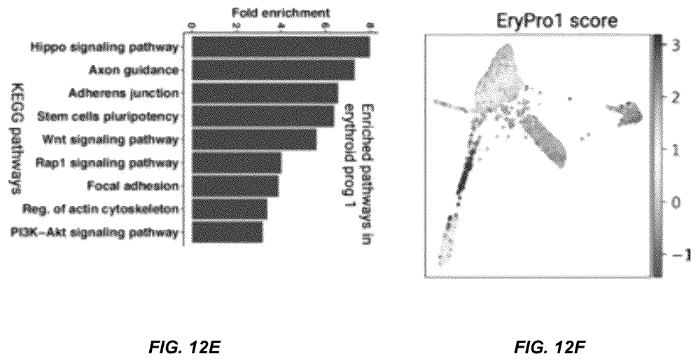

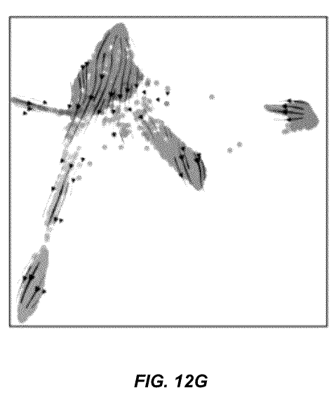

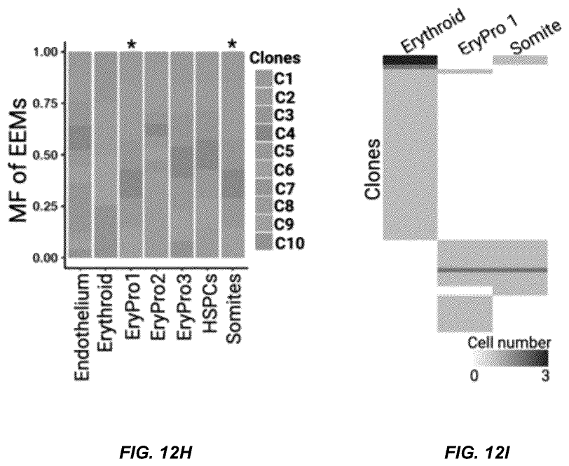

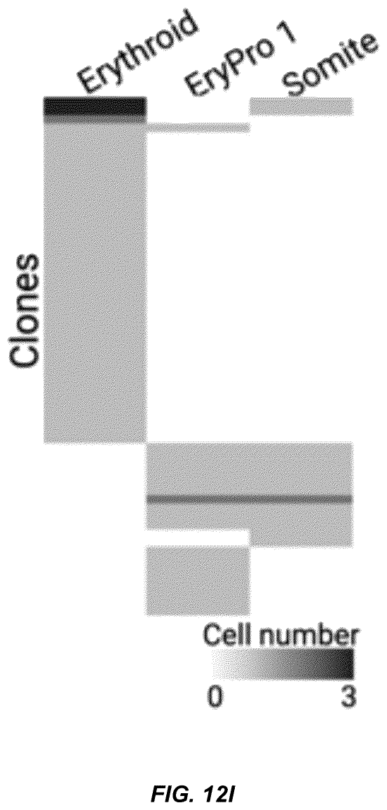

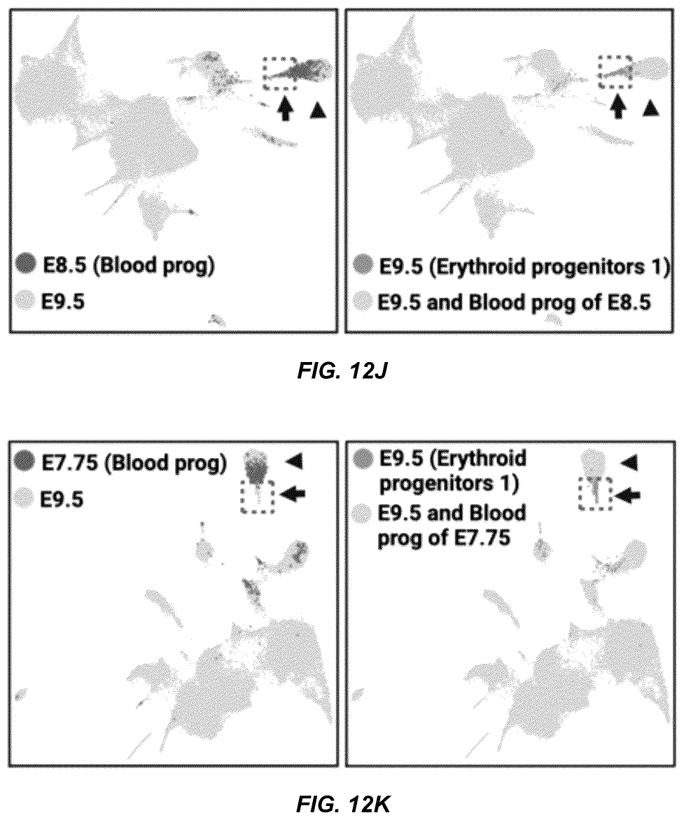

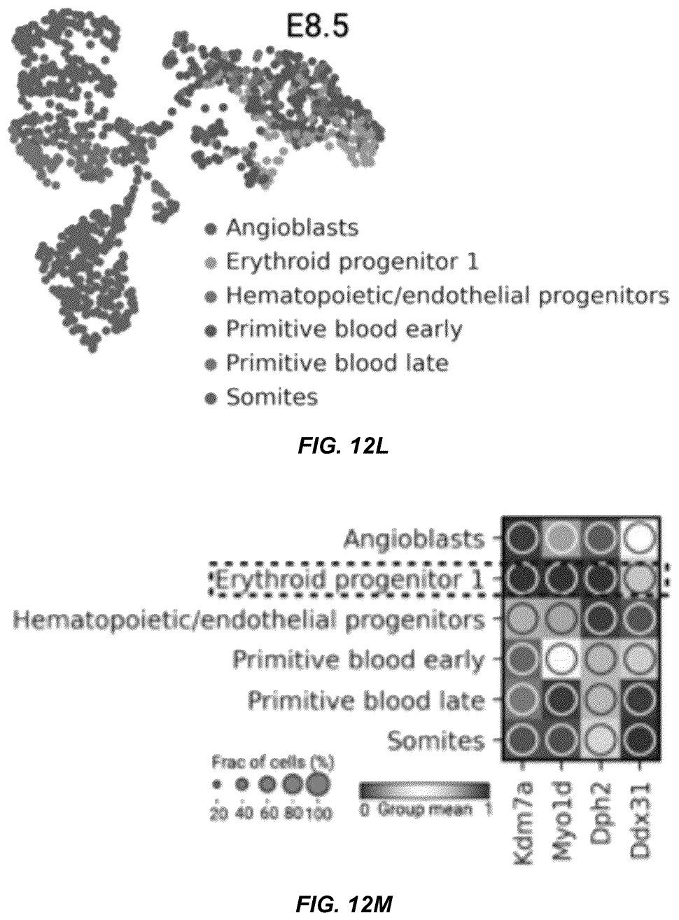

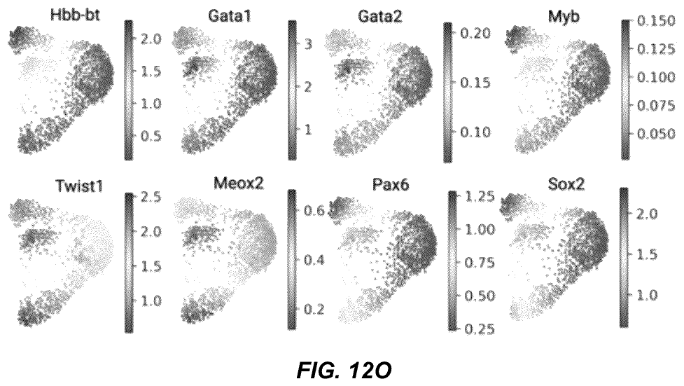

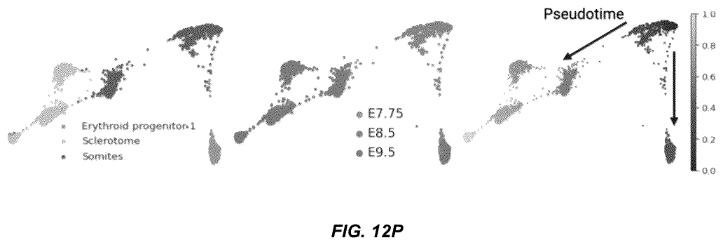

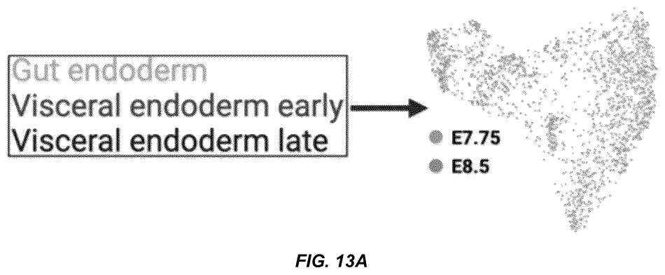

We highlight three unconventional examples that arise from detailed analysis of our data on embryonic development. Lineage analysis at both E8.5 and E9.5 indicated that Erythroid Progenitor 1 (EryPro1) share common ancestry with somite (FIG. 3A). We then reanalyzed somite, endothelium, and hematopoietic cell types, all potential progenitors to EryPro1, and found that EryPro1 did not express yolk sac (Icam2, Krd, and Gpr182), endothelial (Pecam1), and embryonic multipotent progenitor (eMMP) markers (Flt3) (FIGS. 12A-12C) [Patel, S. H., et al., Nature, 2022. 606(7915):747-753]. In contrast, EryPro1 expressed somite-specific markers (Twist1, and Sox11) and showed upregulation of WNT signaling, which comprised an EryPro1-specific gene signature (FIG. 12D-12F). Additionally, RNA velocity, MF of EEMs, and clonal analyses all supported a developmental relationship from somite to EryPro1 (FIG. 12G-12I). Indeed, multiplex HCR-FISH of somite and erythroid markers revealed a cluster of Kit+ erythroid cells in the somite region of the E9.5 embryo (FIG. 3B), supporting a somite-derived erythroid progenitor population. The EryPro1 population is present at E8.5, but not at E7.75 stage, whereas somite cells were observable at E7.75 (FIG. 12J-12M). Gene expression analysis showed some somite cells from E8.5 co-expressed hematopoietic transcription factors (Gata1 and Gata2) and low level of hemoglobin gene (Hbb-bt), implying a cell state transition from somite to EryPro1 (FIG. 12N) [Fujiwara, Y., et al., Blood, 2004. 103(2):583-5; Katsumura, K. R., et al., Blood, 2017. 129(15):2092-2102]. Finally, pseudotime analysis revealed a distinct developmental trajectory from somite to EryPro1, in addition to the expected trajectory from somite to sclerotome (FIG. 12O). Thus, our data supports a somite-derived hematopoietic population during late gastrulation of mammalian development, similar to the zebrafish [54].

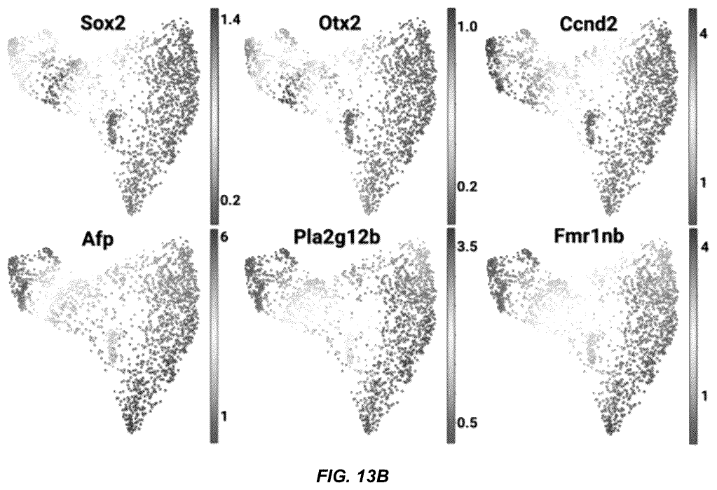

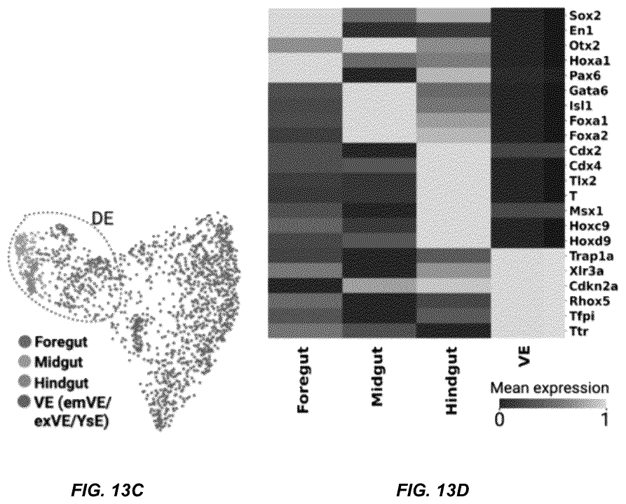

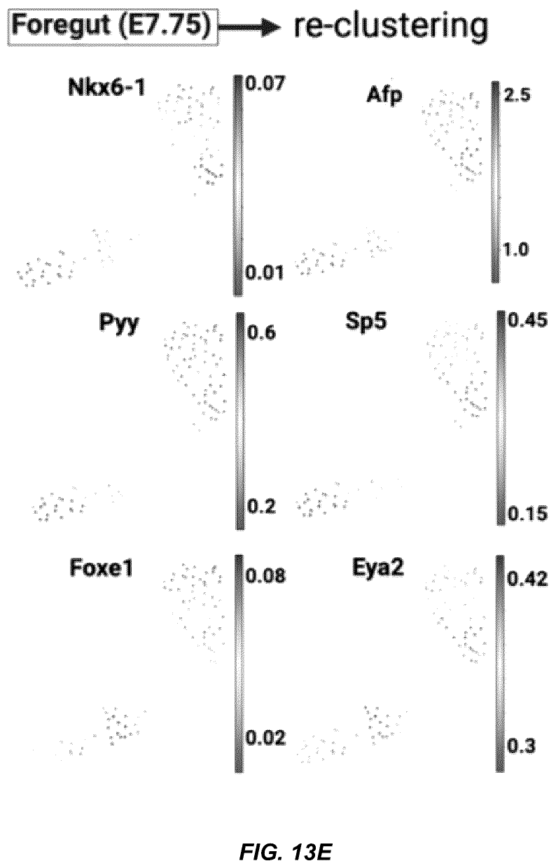

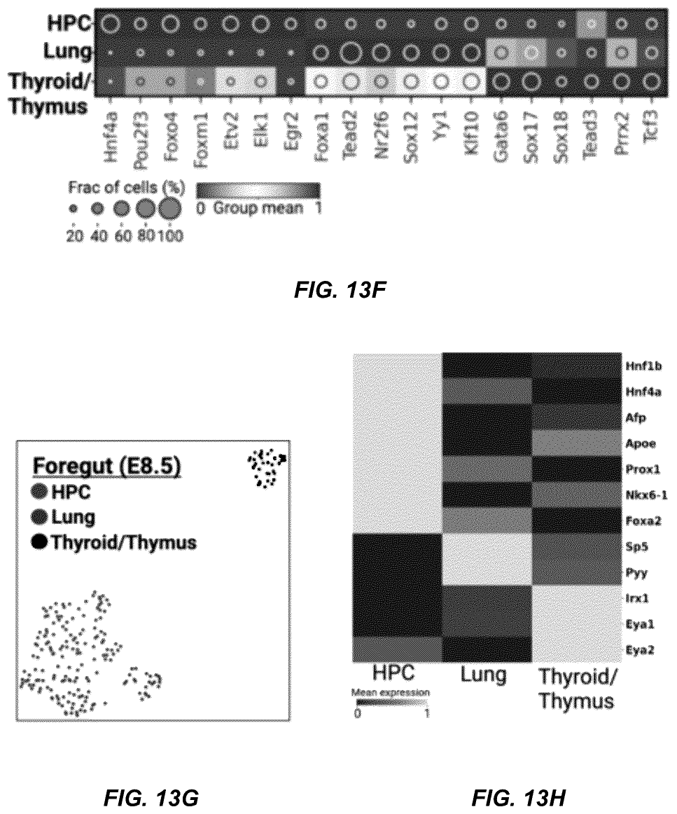

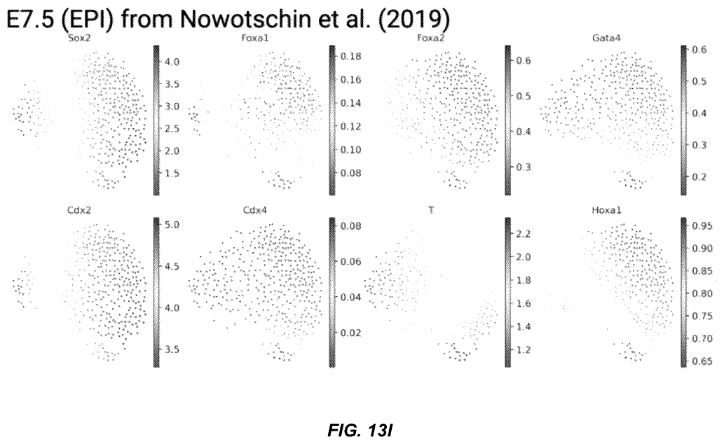

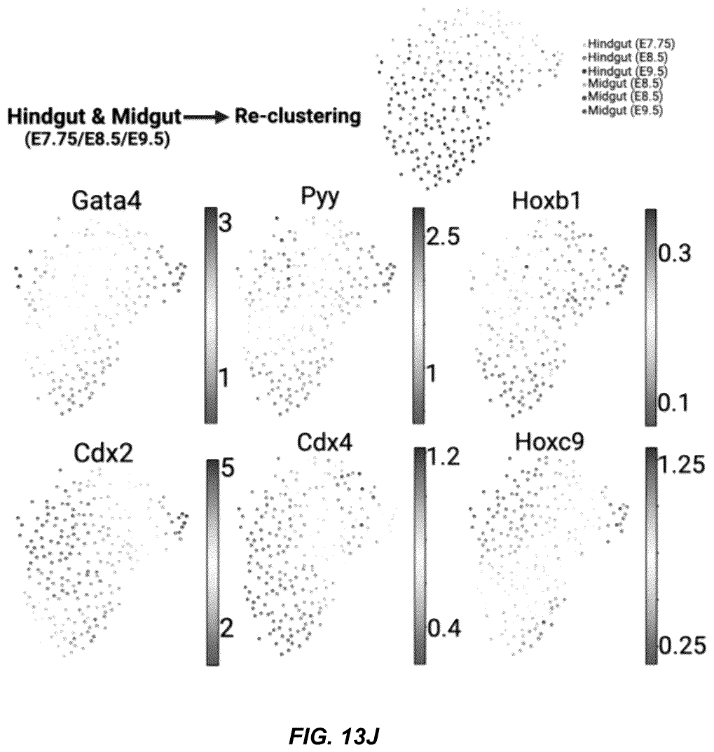

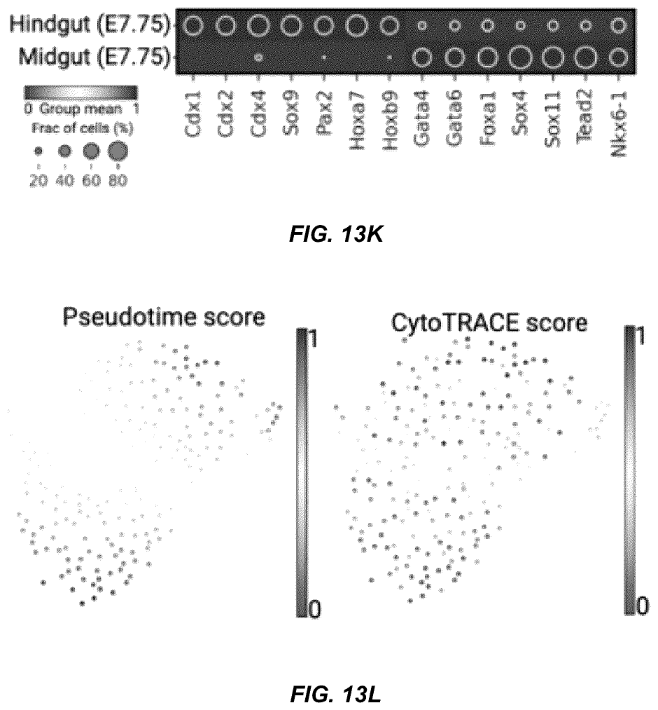

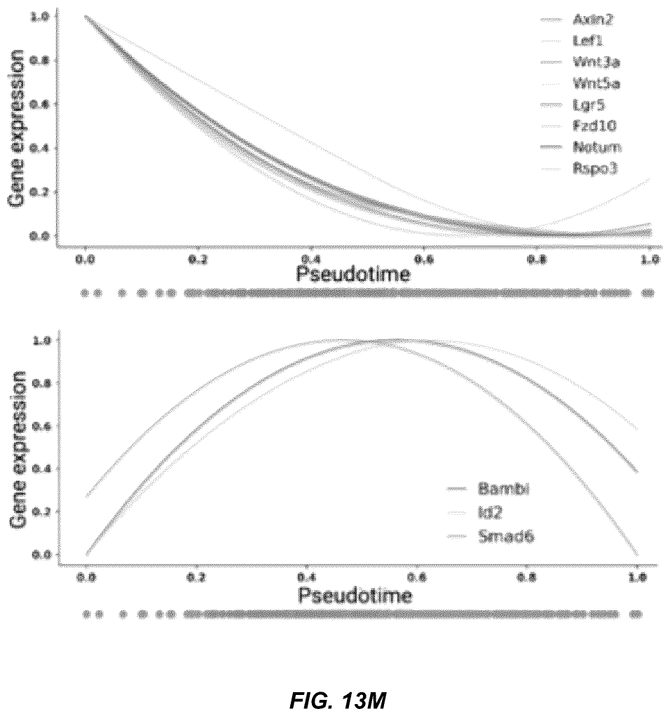

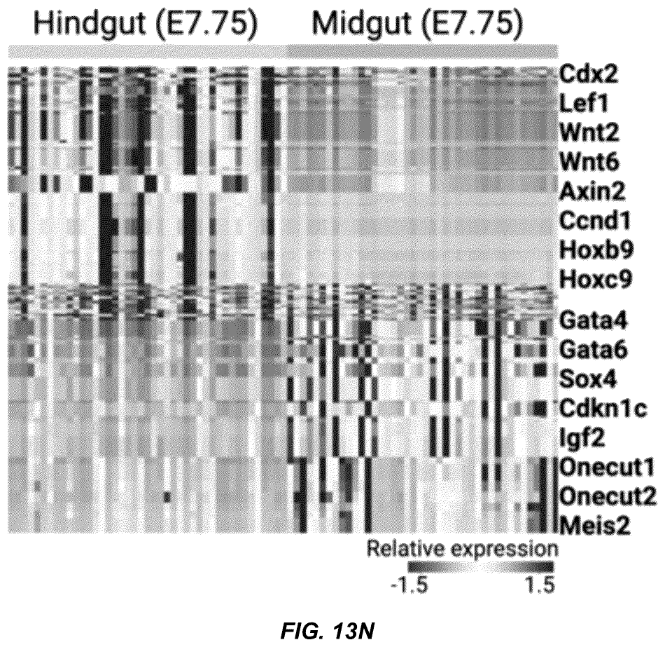

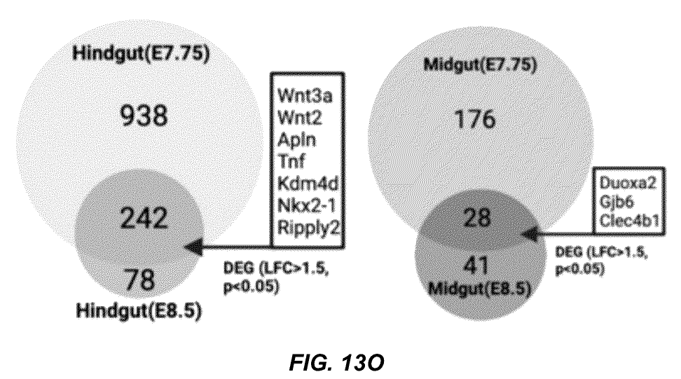

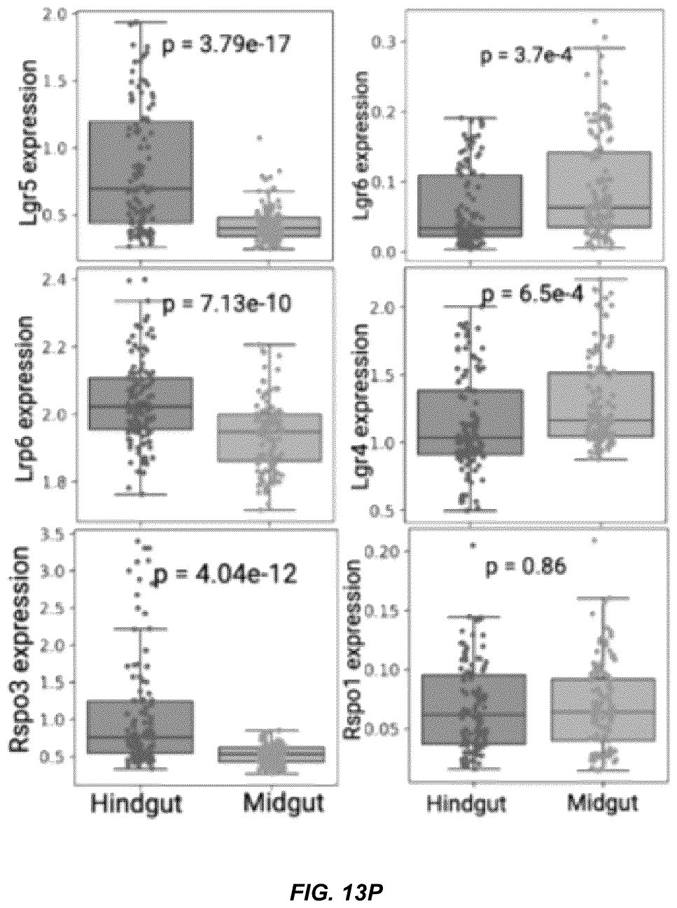

We next sought to understand gut endoderm development in context of regionalization and progenitor specification timing. Endoderm (definitive and visceral) cell populations from E7.75 and E8.5 embryos were plotted together to reveal region-specific markers as early as E7.75, implying regionalization (spatial patterning) at that early time point (FIG. 13A-D). We then focused our analysis on region-specific progenitors of the gut at E7.75. Analysis of foregut population from E7.75 revealed three distinct clusters: hepatopancreatic (HPC) progenitors (Hnf4a+), lung progenitors (Foxa2+), and thyroid/thymus (TT) progenitors (Eya1+) (FIG. 3C). Gene expression, regulon activity, and lineage analysis showed that the HPC population is relatively distinct from lung and TT progenitors (FIGS. 3D-3E, 13E-13F). Similar progenitor populations from the foregut were found at E8.5 (Extended Data FIG. 13G-13H) but not at E7.5 (FIG. 13I), implying precise timing of progenitor specification at E7.75. Analysis of the remaining definitive endoderm populations similarly revealed distinct gene expression between midgut (Gata4, Pyy, and Hoxb1) and hindgut (Cdx2, Cdx4, and Hoxc9) progenitors as early as E7.75 (FIGS. 3F, 13J). Regulon analysis also suggested distinct region specific activities for midgut (Gata4, Foxa1, and Sox11) and hindgut (Cdx2, Sox9, and Pax2) progenitors at this time point (FIG. 13K). Pseudo-time and CytoTRACE analyses resulted in a logical developmental trajectory from E7.75 to E9.5 (FIG. 13L). We found notable region-specific differences in WNT and BMP signaling over developmental pseudo time (FIG. 3M). Significantly higher WNT signaling activity was observed in hindgut compared to midgut progenitors at E7.75 (FIGS. 13N-13O). Consistent with the literature, the WNT target gene Lgr5, a canonical intestinal stem cell marker [Barker, N., et al., Nature, 2007. 449(7165):1003-7], was highly expressed in hindgut [Tsai, Y. H., et al., Cell Mol Gastroenterol Hepatol, 2016. 2(5):648-662.e8], whereas Lgr4 and Lgr6 were expressed in the midgut (FIG. 13P). Our results revealed early differential usage of developmental signaling pathways between progenitors of different regions, supporting an early progenitor specification model during endoderm development [Franklin, V., et al., Mech Dev, 2008. 125(7):587-600; Park, S., et al., Nature, 2021. 597(7876):393-397].

We also examined the lineage relationship between visceral endoderm (VE) and definitive endoderm (DE) during embryonic development. We derived a VE score using reported VE infiltration specific marker genes and showed that the score could accurately mark sorted VE-derived cells (FIG. 14A). Application of this score to our data identified cells that demonstrated high VE-intermixing in the developing hindgut (FIG. 3G, 14B). Unexpectedly, we found that the VE-intermixing score correlated with WNT signaling score and genes (Lgr5, Axin2, and Fzd10) (FIG. 3H, 14C), which was supported by higher Lgr5 expression in sorted VE-derived cells than DE-derived cells (FIG. 14D). Multiplex HCR-FISH showed the presence of cells with co-expressing VE-marker gene Cthro1 and Lgr5 in the hindgut region (dotted line) (FIG. 14E). Lineage analysis using mutational barcodes supported a lineage relationship between hindgut and VE, likely resulting from VE-derived cells mixing into the hindgut during gastrulation (FIG. 3I). This relationship propagates to later development at E9.5, as supported by differential lineage between mid- and hind-gut (FIG. 14F-14G). To determine the role of VE-derived cells in post-gastrulation, we analyzed mutational barcodes on mid- and hind-gut tissue at the E14.5 time point and found that hindgut epithelium have a higher VE intermix score than the midgut epithelium (FIG. 14H-141), consistent with the results above. We then assessed the ability of these cells to contribute to epithelial development by performing a ‘parent-childless’ clonal analysis using a reported approach [Bowling, S., et al., Cell, 2020. 181(6):1410-1422.e27] (FIG. 14j). VE-derived cells have a high parent clone fraction, implying that they have a higher potential to give rise to progeny (FIG. 14K). Mutation density analysis also demonstrated VE-derived cells have accumulated more divisions at E14.5 compared to other definitive endoderm-derived cells, demonstrating their activity during post-gastrulation (FIG. 14L). Finally, we performed mutational barcode analysis of foregut, midgut, and hindgut derived tissues at the adult stage and found that hindgut-derived tissues maintain a separate lineage branch from midgut and foregut derived tissues even into the adult (FIG. 14M). Thus, our data support previous reports of VE-derived cells intermixing with definitive endoderm (FIG. 14N) predominantly in hindgut [36], and their potential contribution to gut epithelial development [Chan, M. M., et al., Nature, 2019. 570(7759):77-82; Pijuan-Sala, B., et al., Nature, 2019. 566(7745):490-495; Nowotschin, S., et al., Nature, 2019. 569(7756):361-367; Ibarra-Soria, X., et al., Nat Cell Biol, 2018. 20(2):127-134; Peng, G., et al., Nature, 2019. 572(7770):528-532; Kwon, G. S., et al., Dev Cell, 2008. 15(4):509-20].

### Identification of an Early Embryonic Progenitor of Gut Epithelium:

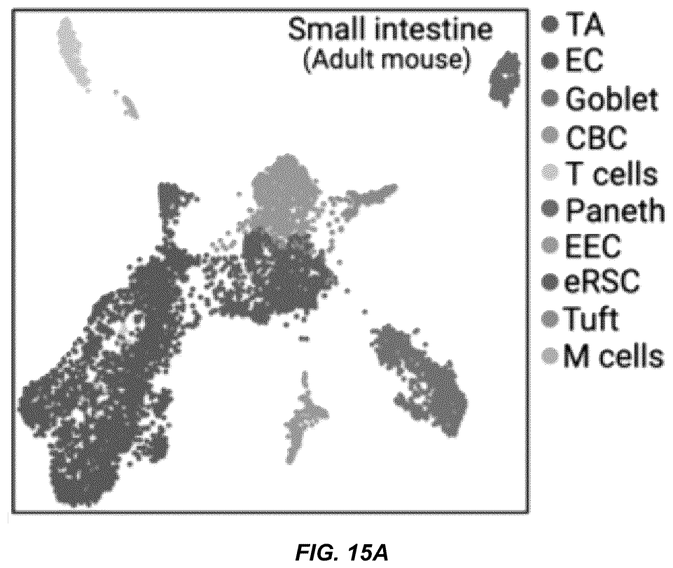

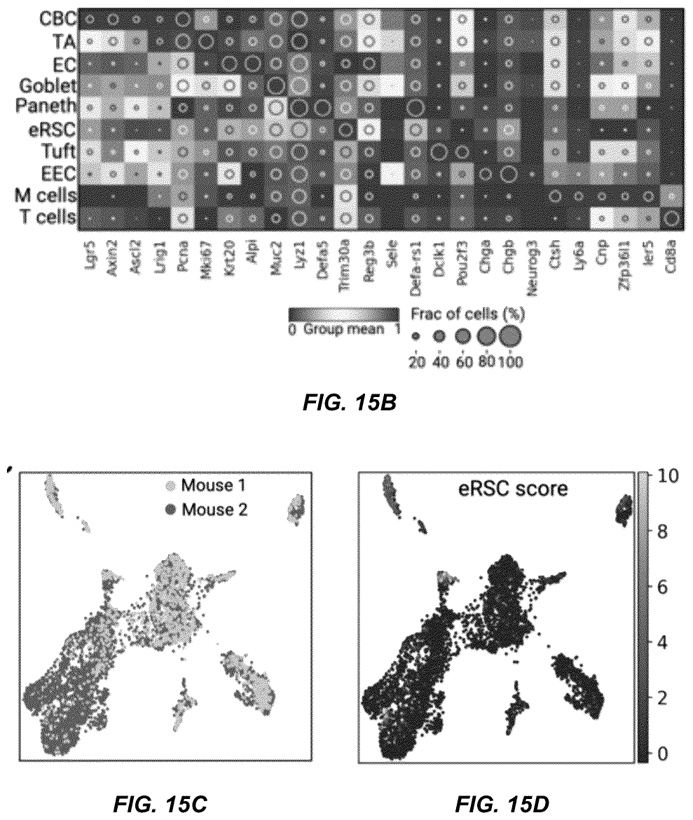

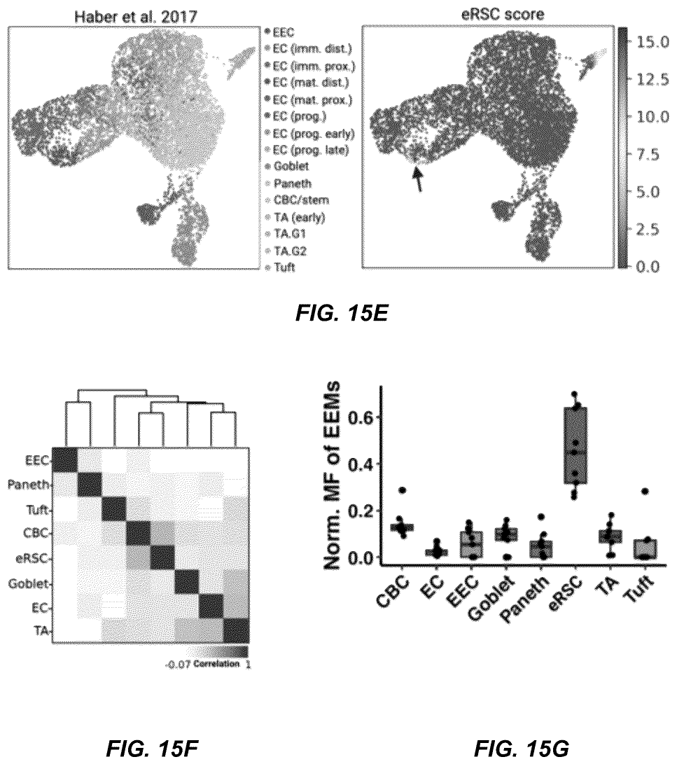

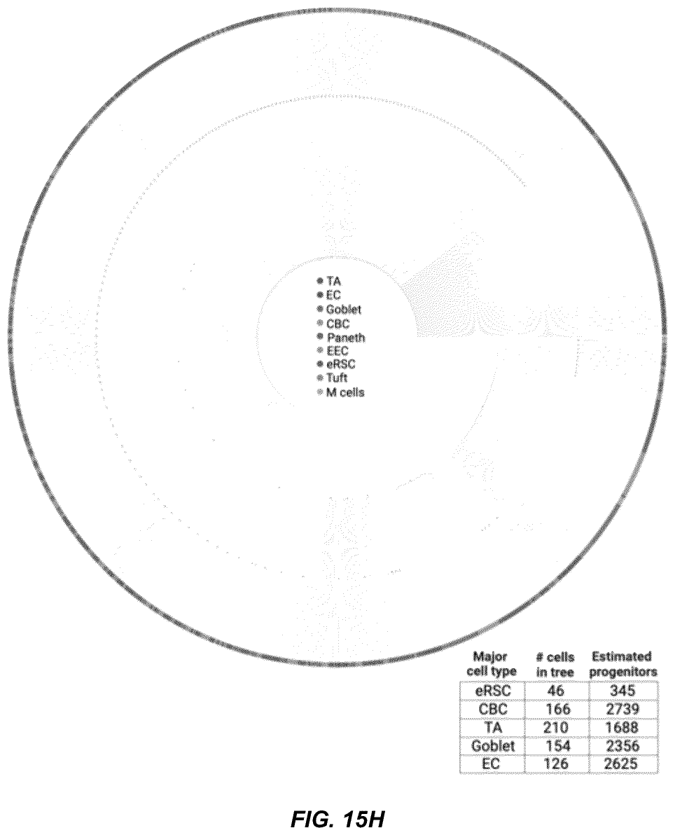

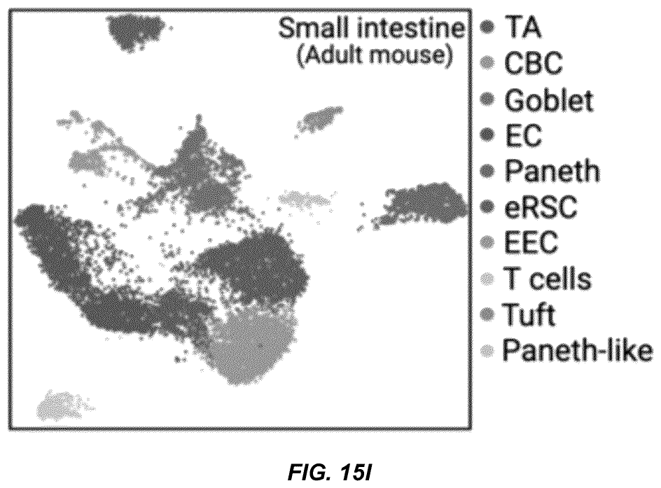

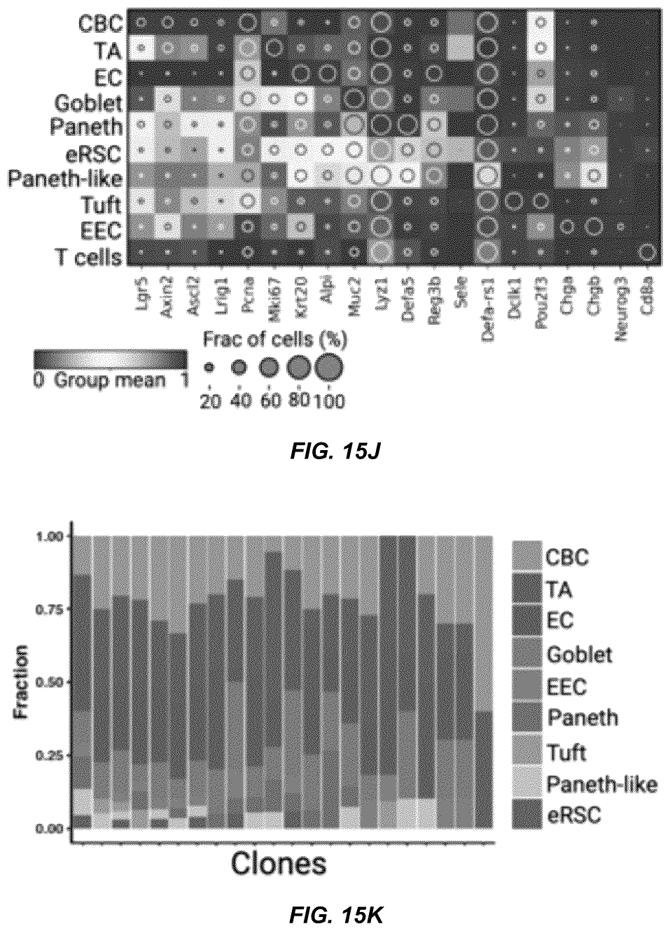

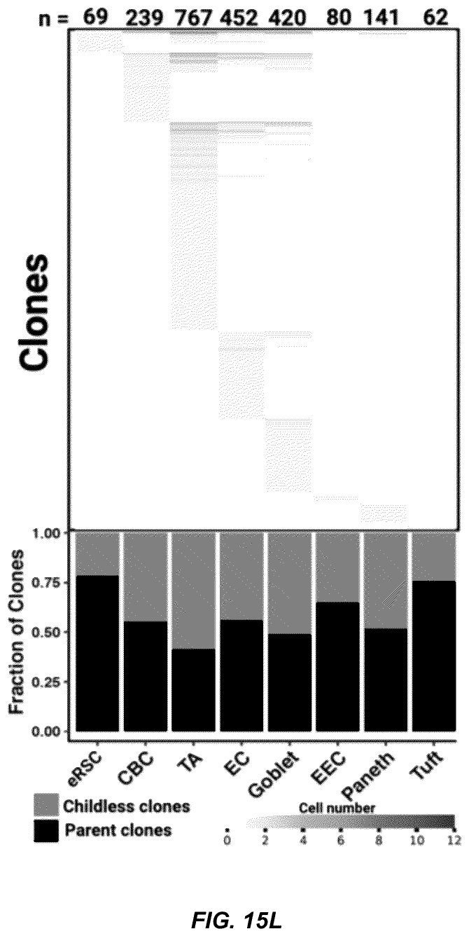

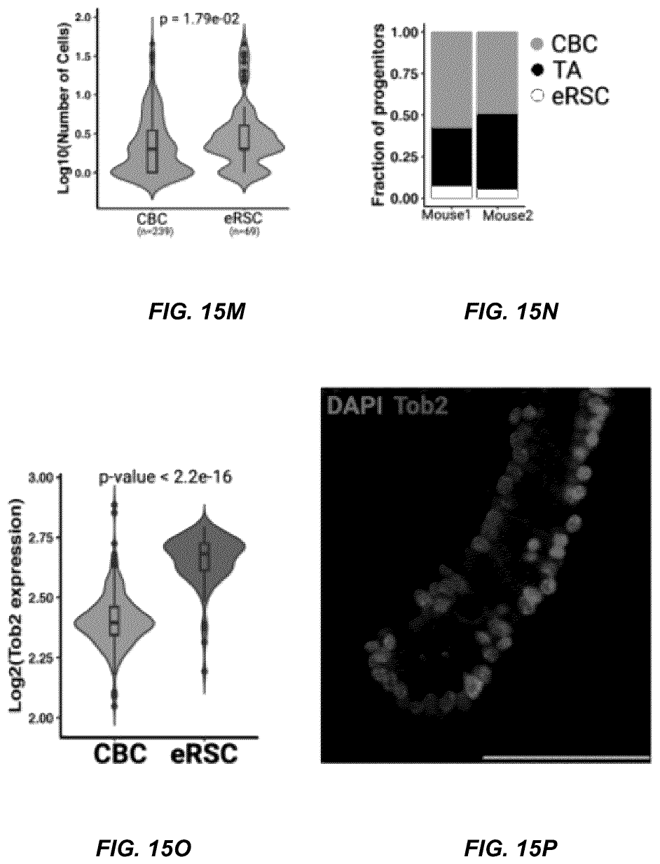

NSC-seq applied to the adult gut tissues reproducibly identified a unique cell population related to enterocytes in the small intestine, which we labeled embryonic revival stem cells (eRSC) (FIG. 15A-15C). A gene signature derived from this cell population was also able to identify the same cells in another publicly available dataset (FIG. 15D-15E). Mutational lineage analysis demonstrates a developmental relationship between crypt-based columnar cells (CBC) and eRSC cells, indicating that they potentially derive from each other (FIG. 15F). However, eRSCs exhibit a significantly higher mosaic fraction, implying that they are derived from much earlier cell generations compared to CBCs, which develop relatively later during fetal intestinal development [Guiu, J., et al., Nature, 2019. 570(7759):107-111] (FIG. 15G). A smaller number of progenitors that give rise to these cells inferred from single-cell lineage tree topology (FIG. 15H) supports their earlier specification stemming from fewer progenitors available at earlier development. Clonal contribution analysis using hgRNA mutations demonstrates that the eRSCs population possesses a larger clone size, and thus, contributes more progenies to the intestinal epithelium than CBCs (FIG. 3J-3K). This result was repeatable, supporting that the Ersc population acts as a stem/progenitor-like population during intestinal development (FIGS. 15I-15N). We selected Transducer of Erbb2.2 (Tob2) as marker of eRSC cells and found that they are located at the bottom of the adult small intestinal crypt via antibody staining (FIGS. 15O-15P). These data support that eRSCs can act as stem/progenitor-like cells to populate the gut during embryogenesis, in contrast to the limited contribution of the CBC population at that time [Guiu, J., et al., Nature, 2019. 570(7759):107-111].

### Clonal Analysis of Colorectal Precancers:

Tumours are often thought to form through aberrant developmental gene programs [Egeblad, M., et al. Dev. Cell 2010 18:884-901]. An unresolved issue in colon cancer is whether tumours arise from a single stem cell or from multiple progenitor cells to result in complex tissue systems. Thus, we utilize NSC-seq, in approaches akin to what we used to study developmental origins, to investigate the origins of tumourigenesis in the gut. The prevailing model, with support from human colorectal cancer data, is the monoclonal model, in which a tumour is initiated from a single stem cell [Fearon, E. R., et al. Science 1987 238:193-197]. However, selection and clonal sweeps that occur in advanced cancers tend to erase clonal histories occurring earlier in tumourigenesis [Williams, M. J. et al. Nat. Genet. 2016 48:238-244]. Furthermore, lineage-tracing studies in the mouse have shown that some tumours can be initiated from multiple ancestors, resulting in tumours with multiple lineage labels [Thorsen, A. S. et al. Dis. Model. Mech. 2021 14: dmm046706]. We thus applied single-cell barcode tracking to delineate clonality during intestinal tumour initiation in ApcMin/+ mice, in which tumourigenesis occurs as a result of random mutations inactivating the second allele of Apc.

We found that these tumours were composed of both normal and tumour-specific cells, similar to human adenomas in a previous study (FIG. 16) [Chen, B. et al. Cell 2021 184:6262-6280]. Evaluation of tumour-specific cells using NSC-seq demonstrated increased proliferation signature, stemness, fetal gene expression (Marcksl1) and clonal contribution compared with normal CBCs (FIG. 16), consistent with the transformed features of these cells. Examination of phenotypically normal cells within the tumour showed normal-like progenies of tumour-specific cells, which can be distinguished from their normal counterparts by their higher barcode mutation densities and shared barcode mutation profiles with tumour cells (FIG. 16). Interestingly, these progenies consisted of enterocytes and Paneth cells, consistent with Wnt-restricted aberrant differentiation of intestinal tumour cells [Schepers, A. G. et al. Science 2012 337:730-735]. To delineate clonality, we first used shared barcode mutations in lymphocytes, demonstrating that tumour-infiltrating lymphocytes had expanded clonally compared with peripheral blood lymphocytes, which were mostly polyclonal (FIG. 16). A similar analysis showed three founder clones within tumour-specific cells (FIG. 4A). The three clones were distinct in many characteristics, including mutation density, clonal contribution, biased differentiation and gene expression signatures (FIG. 4B, 16). More importantly, single-cell phylogenetic analysis showed independent tumour founder clones arising from distinct normal epithelial ancestors (FIG. 16). Next, we performed whole-exome sequencing (WES) of 13 murine intestinal tumours to assess the number of Apc mutations. Loss-of-function mutations in both APC alleles that result in Wnt pathway activation are considered the initiating event in the majority of sporadic human colorectal tumours [Fearon, E. R. et al. Cell 1990 61:759-767]. Thus, the number of unique Apc mutations can be used to assess clonality during intestinal tumour initiation [Thirlwell, C. et al. Gastroenterology 2010 138:1441-1454]. In a diploid genome, a monoclonally initiated tumour should present at most two unique Apc mutations that lead to loss of function of both alleles, given that there is no selective advantage for additional mutations. We found that five of the 13 murine intestinal tumours had three or more unique mutations in the Apc gene, implying multiple founder clones (FIG. 4C). Moreover, around 40% of murine tumours showed evolutionary selection pressure comparable to human adenomas (see below and FIG. 16). The normal cell of origin of tumour cells can also be examined by early embryonic clonal intermixing using barcode mutations in both tumour and adjacent normal tissues from the same mouse [Thliveris, A. T. et al. Cancer Prev. Res. (Phila.) 2011 4:916-923]. Early embryonic clonal intermixing was seen in four out of five mouse polyclonally initiated tumours (FIG. 16), indicating that barcode mutations used.

### Example 2: Scalable Single-Cell Pooled CRISPR Screens with Conventional Knockout Vector Libraries

### Results

CRISPR screens systematically perturb targeted genes and assess functional outcomes through phenotypic changes in mammalian cells, enabling unbiased discovery of gene functions, regulatory network organization, and genotype-phenotype relationships [Bock, C., et al., Nat Rev Methods Primers, 2022. 2(1); Shalem, O., et al., Science, 2014. 343(6166):84-87]. Substantial progress has recently been made in implementing high-throughput CRISPR screening at single-cell resolution, which utilizes gene expression of individual cells with corresponding CRISPR-perturbed genes as phenotypic outputs. Many of such approaches, such as Perturb-seq, utilize specialized vectors that allow the indirect capture of sgRNAs by standard 3′-end single-cell RNA-seq (scRNA-seq) methods [Dixit, A., et al., Cell, 2016. 167(7):1853-1866.e17; Adamson, B., et al., Cell, 2016. 167(7):1867-1882.e21; Datlinger, P., et al., Nat Methods, 2017. 14(3):297-301; Jaitin, D. A., et al., Cell, 2016. 167(7):1883-1896.e15]. Direct 3′ sgRNA methods have recently been reported but they also require custom modification of plasmid libraries [Replogle, J. M., et al., Nat Biotechnol, 2020. 38(8):954-961; Wessels, H. H., et al., Nat Methods, 2023. 20(1):86-94]. The use of these specialized vectors limits the scale and flexibility of genetic screens, since they are incompatible with existing genomes-scale knockout (KO) libraries [Bock, C., et al., Nat Rev Methods Primers, 2022. 2(1); Sanson, K. R., et al., Nat Commun, 2018. 9(1):5416; Hart, T., et al., G3 (Bethesda), 2017. 7(8):2719-2727]. Moreover, specialized vectors are susceptible to sgRNA-barcode swapping events due to lentiviral template switching [Hill, A. J., et al., Nat Methods, 2018. 15(4):271-274].

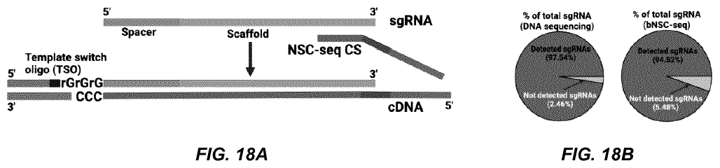

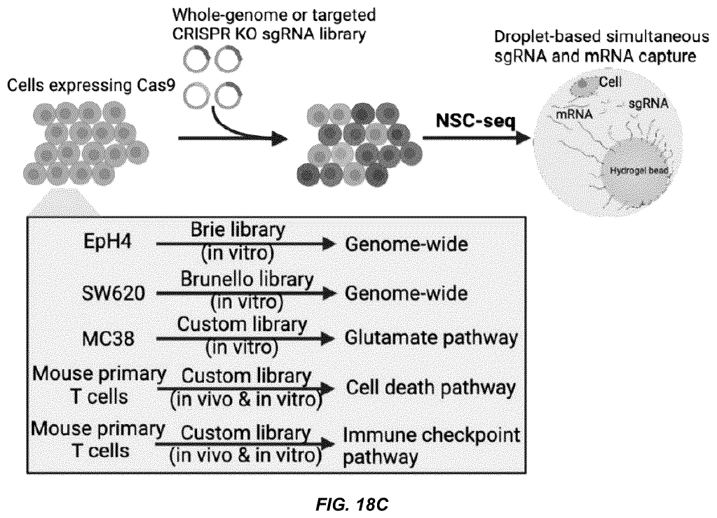

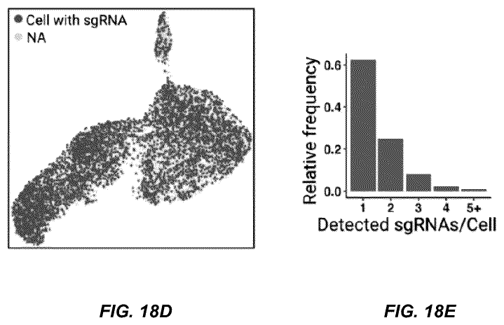

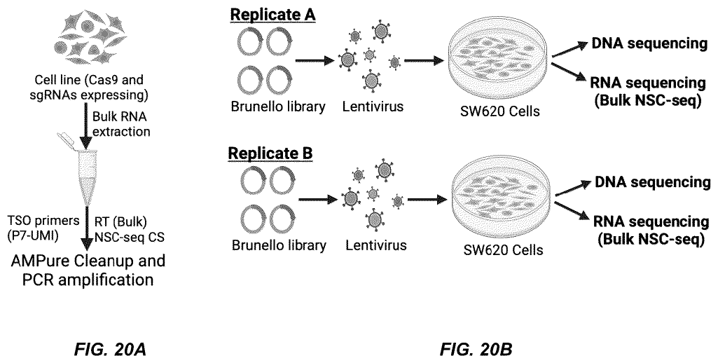

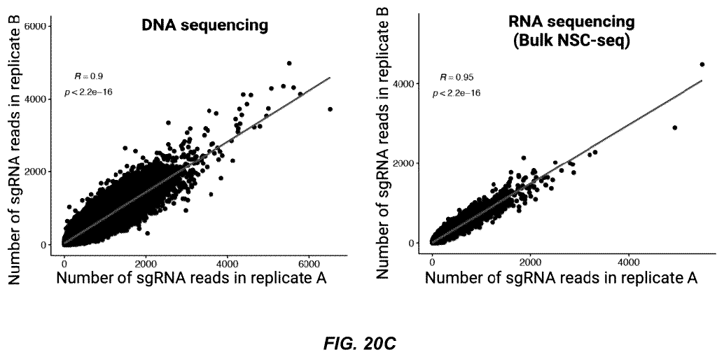

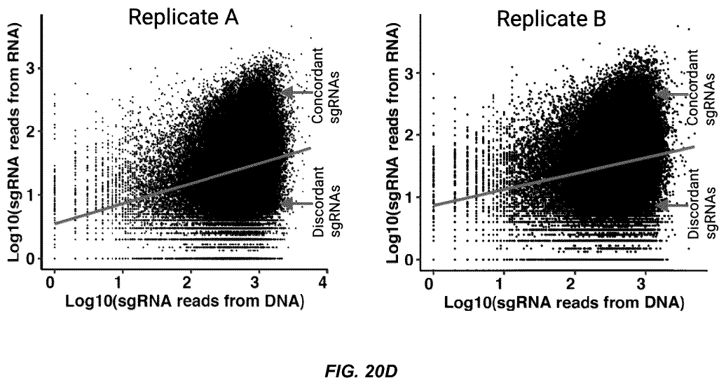

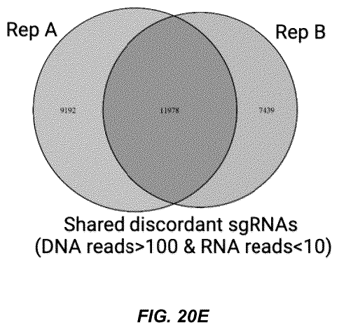

Here, we present a custom 3′ single-cell capture platform, called Native sgRNA Capture and sequencing (NSC-seq), that enables flexible and multi-purpose single-cell CRISPR screening using existing KO vector libraries. To capture non-polyadenylated sgRNAs [Jinek, M., et al., Science, 2012. 337(6096):816-21], we designed an inDrops-compatible capture sequence (CS) that binds to the canonical scaffold of gRNAs to initiate direct reverse transcription (RT) of the captured sgRNA. A primer sequence was then added to the 3′-end of the cDNA via template switching to facilitate downstream library amplification (FIG. 18A). As proof of concept, we assessed sgRNA detection efficiency and reproducibility through RNA-based readouts using a human colorectal cancer (CRC) cell line SW620 with the Brunello library (FIGS. 20A-20B). The NSC-seq approach exhibited comparable performance to traditional DNA-based detection (FIGS. 1B, 20C). Importantly, a subset of sgRNAs exhibited discordance between DNA- and RNA-based detection, with approximately 60% of these sgRNAs shared between biological replicates (FIGS. 20D-20E). This result suggests that the discordance may not have resulted from random technical artifacts but from inherent biases in the expression of different sgRNAs under the U6 promoter [Gao, Z., et al., Transcription, 2017. 8(5):275-287; Ma, H., et al., Mol Ther Nucleic Acids, 2014. 3(5): e161]. The CS can be used to capture a wide range of gRNAs with a similar scaffold sequence (sgRNAs, hgRNAs, and stgRNAs) [Perli, S. D., et al. Science, 2016. 353(6304); Kalhor, R., et al. Nat Methods, 2017. 14(2):195-200; Kalhor, R., et al., Science, 2018. 361(6405)]. In a companion study, we used NSC-seq to directly capture hgRNAs, enabling in vivo recording of lineage and temporal events during mammalian development and tumorigenesis [Islam, M., et al., bioRxiv. 2023 Dec. 19:2023.12.18.572260]. The capture efficiency of sgRNA at single-cell resolution using NSC-seq can be as high as 95% as shown using the mouse breast epithelial cell line EpH4 with the Brie library. With these performance parameters, we then performed single-cell pooled CRISPR screens using five individual KO vector libraries to reveal effects of genetic perturbation on transcriptomic changes [Dixit, A., et al., Cell, 2016. 167(7):1853-1866.e17] in vitro and in vivo (FIG. 18C).

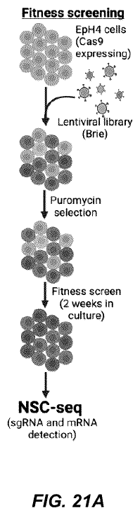

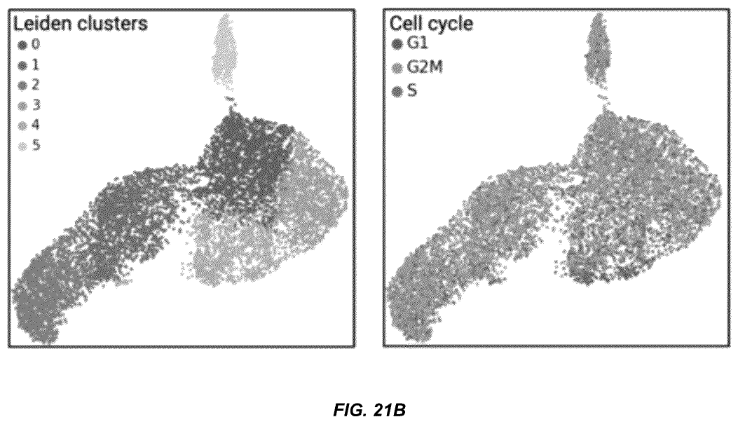

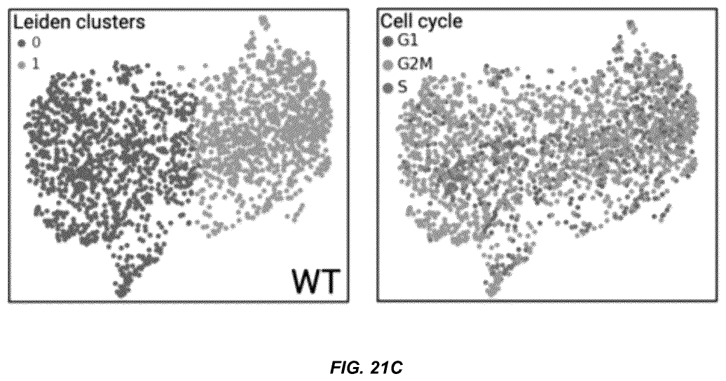

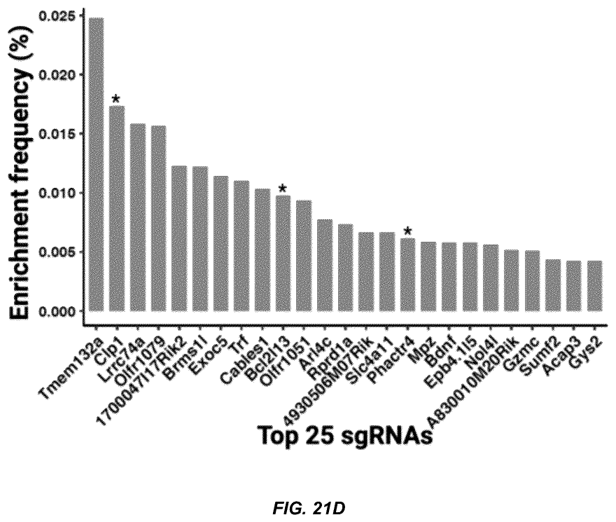

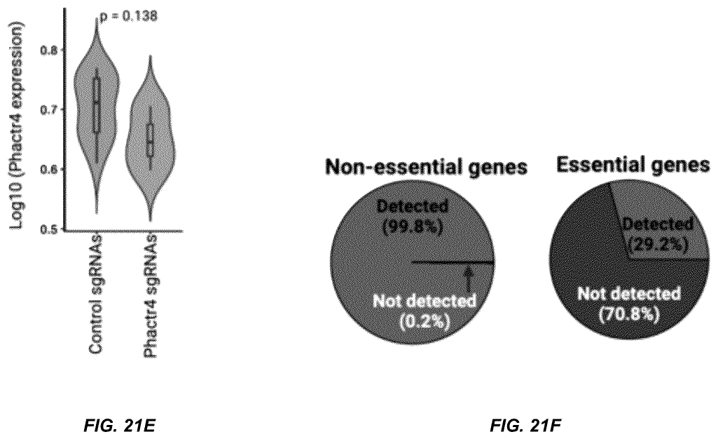

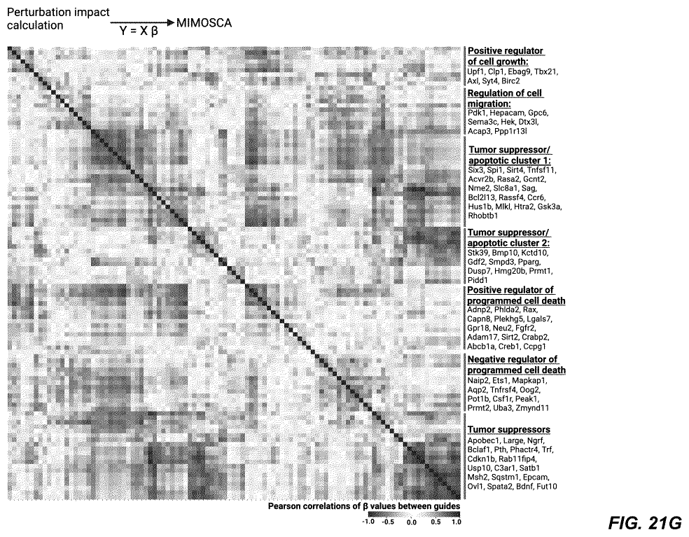

We conducted a pooled single-cell fitness screen using the EpH4 cell line with the Brie library, with the majority of captured cells having a single gRNA detected (FIG. 18D-18E). While the wild type (WT) cell line was homogeneous, CRISPR-perturbed cells displayed heterogeneity in transcriptome space, reflecting the efficacy of the various genetic perturbations on changing gene expression (FIGS. 21A-21C). Our analysis revealed the enrichment of tumor suppressor and/or apoptotic gene-targeting sgRNAs, including Bcl2113 and Phactr4, to be among the top 25 enriched sgRNAs (FIGS. 21D-21E) [Solimini, N. L., et al., Proc Natl Acad Sci U SA, 2013. 110(5): E407-14; Kataoka, T., et al., J Biol Chem, 2001. 276(22):19548-54]. We also found that NSC-seq detected the sgRNAs for all reported non-essential gene-targeting sgRNAs, whereas sgRNAs targeting essential genes were not picked up as readily, given that KO of essential genes leads to the elimination of targeted cells from the pool (FIG. 21F) [Hart, T., et al., Cell, 2015. 163(6):1515-26]. We then conducted a more in-depth analysis using a mixed linear model [Dixit, A., et al., Cell, 2016. 167(7):1853-1866.e17], revealing seven functional modules dominated by tumor suppressor and/or apoptotic genes (FIG. 21G). Perturbed genes of the same pathway generated the same effects on gene expression, and thus were organized into the same functional module. For instance, Sqstm1, Msh2, Bclaf2, and Spata2 all belong to the “Programmed Cell Death” pathway according to GO and were all part of module 7. Other genes, although known generally for their antiproliferative effects, such as Cdkn1b and Bcl2113, were found to operate through different pathways. Our results reveal distinct pathways that affect cell survival and proliferation, and facilitate the identification of the potential functions of less-characterized genes through associations with the functional modules.

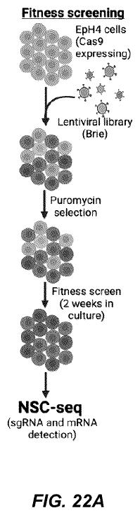

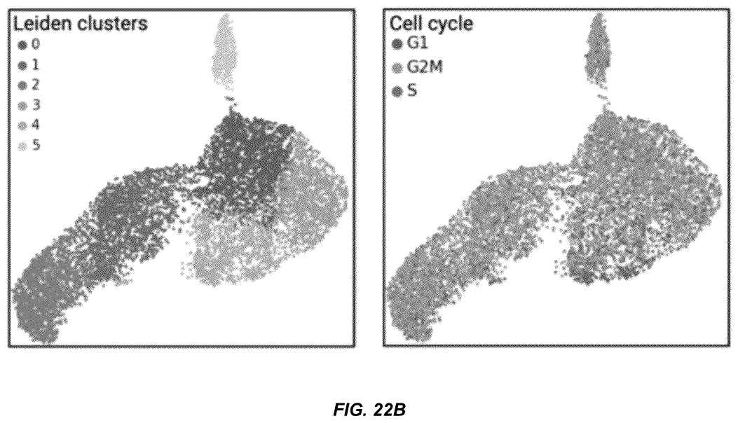

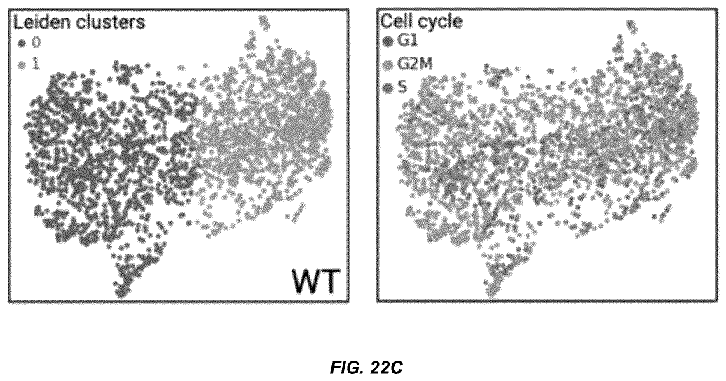

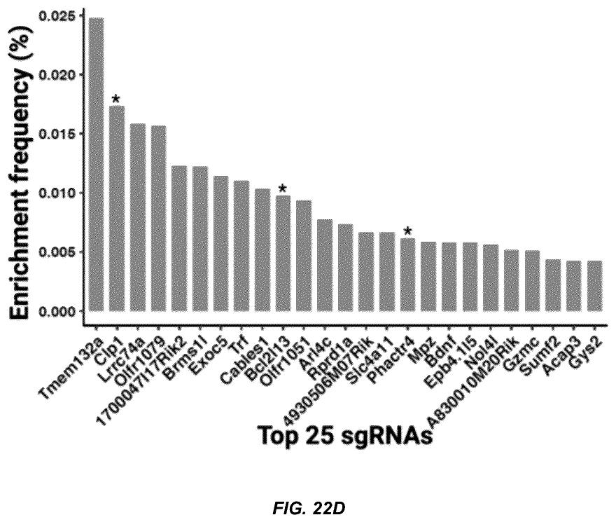

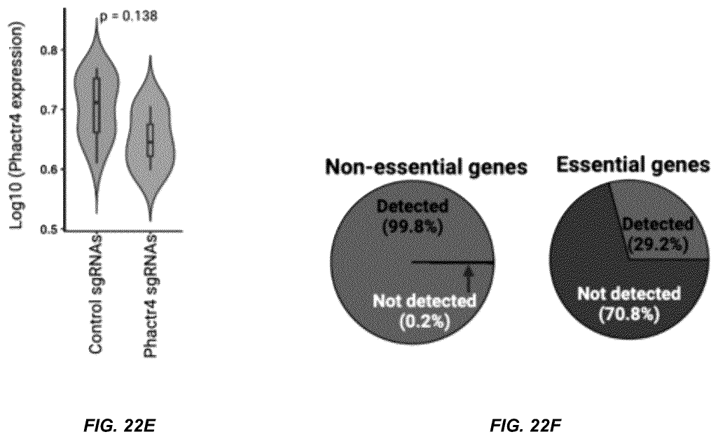

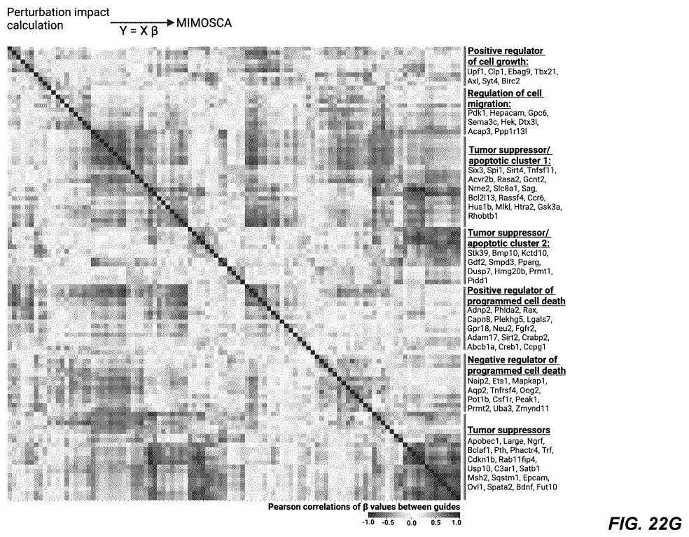

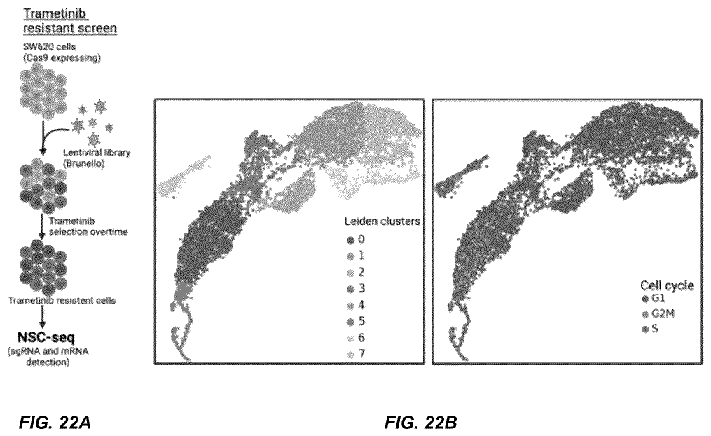

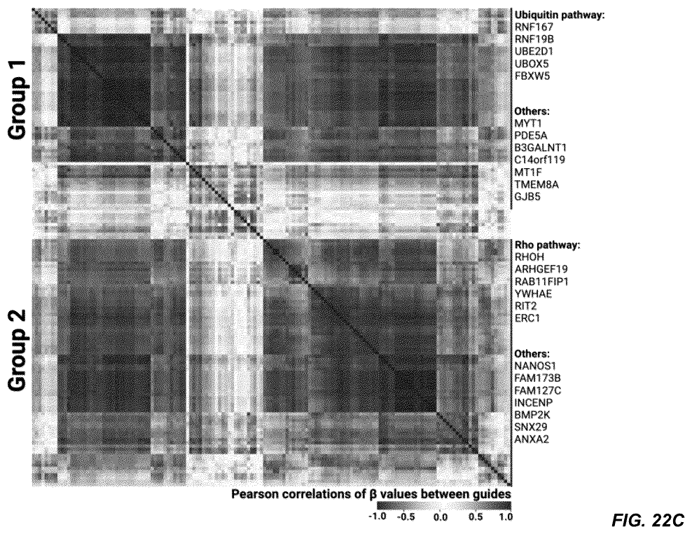

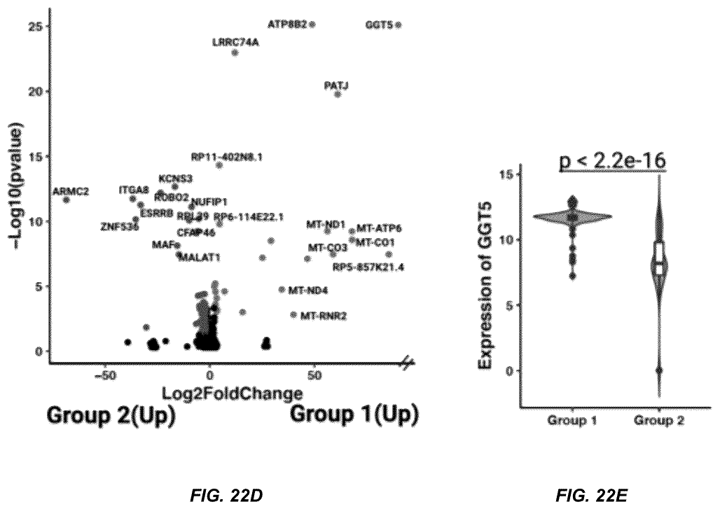

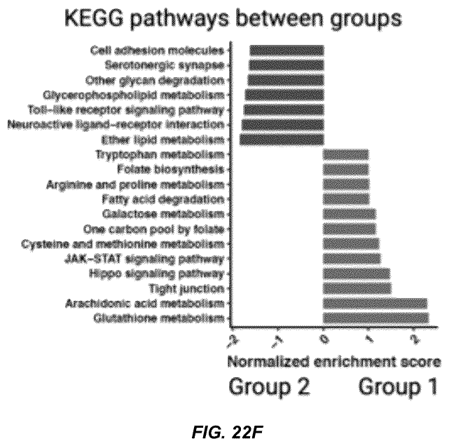

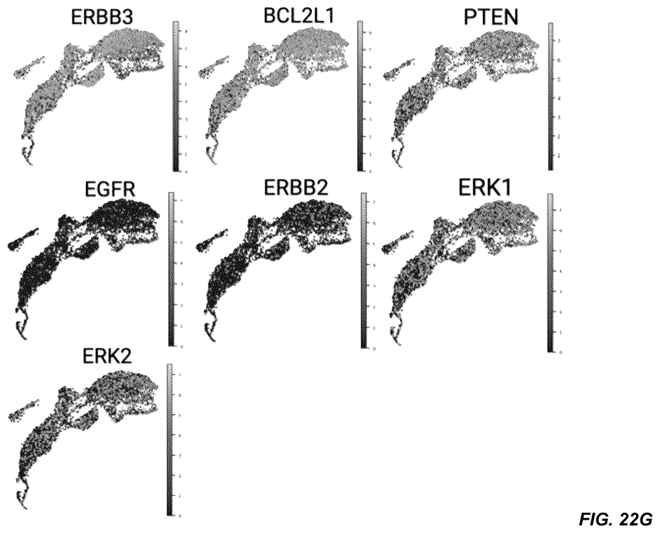

Next, we conducted a Trametinib resistance screen in the SW620 cells with the Brunello library (FIG. 22A) [Wu, P. K. and J. I. Park. Semin Oncol, 2015. 42(6):849-62]. Trametinib-resistant cells were largely found in a quiescent state (G1), a common mechanism for cancer cells to escape MEK1/2 inhibition (FIG. 22B) [Alarcón, T. and H. J. Jensen. J R Soc Interface, 2011. 8(54):99-106; Borst, P., Open Biol, 2012. 2(5):120066]. It has been reported that MEK inhibition reduces cellular growth via multiple mechanisms, including the induction of MYT1 hypo-phosphorylation [Villeneuve, J., et al., Embo j, 2013. 32(1):72-85] and increasing the levels of PDE5A [Arozarena, I., et al., Cancer Cell, 2011. 19(1):45-57]. Accordingly, enrichment of both MYT1- and PDE5A-targeting sgRNAs were found amongst resistant cells. We further analyzed genetic perturbations that modified drug resistance, revealing two regulatory modules with enrichment of ubiquitin pathway or Rho pathway genes (FIG. 22C) [Narayanan, S., et al., Drug Resist Updat, 2020. 48:100663; Deng, L., et al., Signal Transduct Target Ther, 2020. 5(1):11; Kreider-Letterman, G., et al. Eur J Cell Biol, 2022. 101(2):151209]. Differential gene expression analysis revealed distinct metabolic gene enrichment, especially overexpression of the glutathione metabolism gene GGT5 in the ubiquitin module (FIG. 22D-22F). We also found overexpression of ERBB3 in resistant cells that resulted in PI3K/AKT activation due to MEK inhibition-mediated negative feedback on ERBB receptors (FIG. 22G) [Turke, A. B., et al., Cancer Res, 2012. 72(13):3228-37; He, Y., et al., Signal Transduct Target Ther, 2021. 6(1):425]. Thus, our results reveal multi-factorial trametinib resistance mechanisms and a possible actionable pathway through the PI3K/AKT pathway for KRAS mutant colorectal cancer cells to escape MEK1/2 inhibition [Vander Velde, R., et al., Nat Commun, 2020. 11(1):2393].

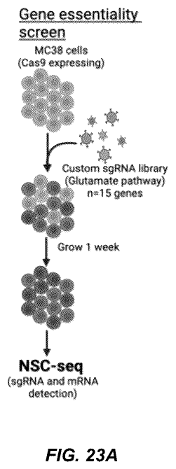

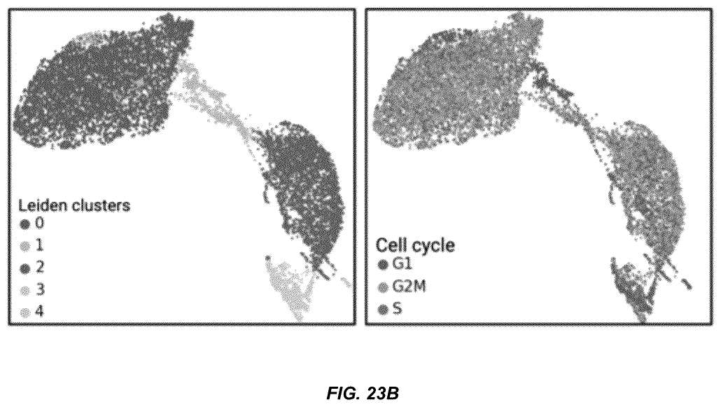

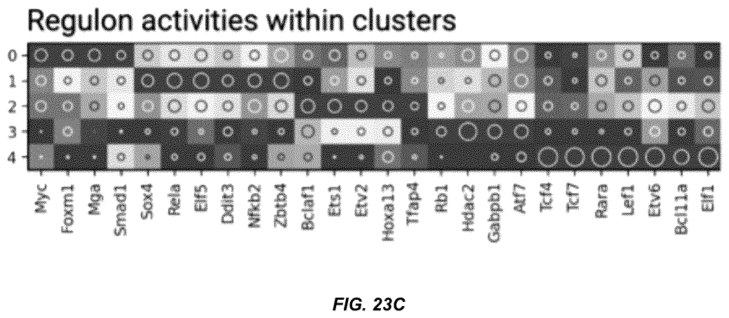

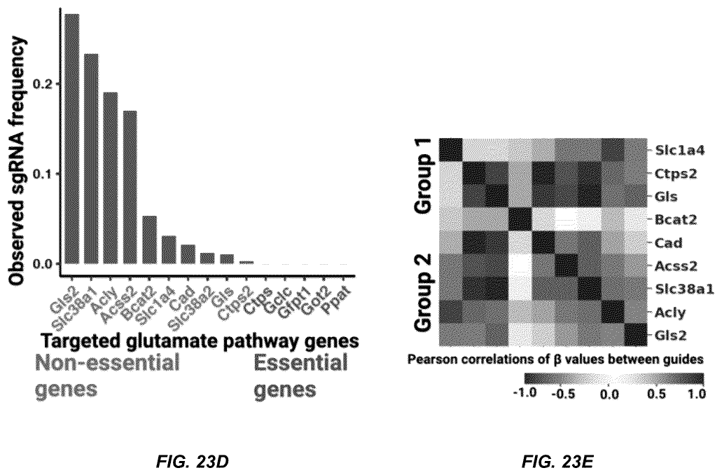

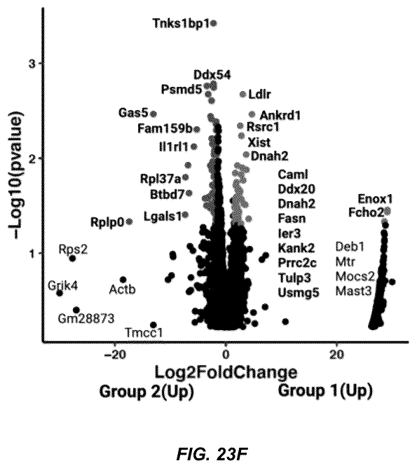

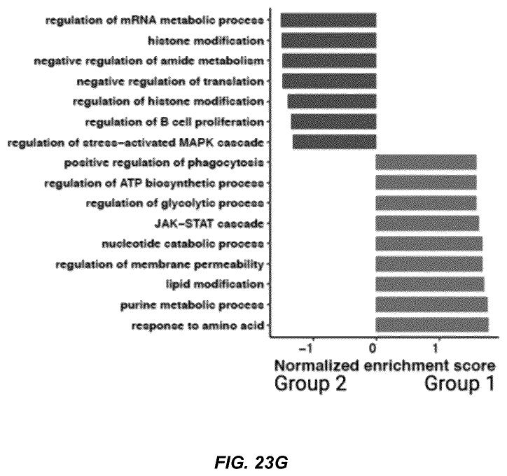

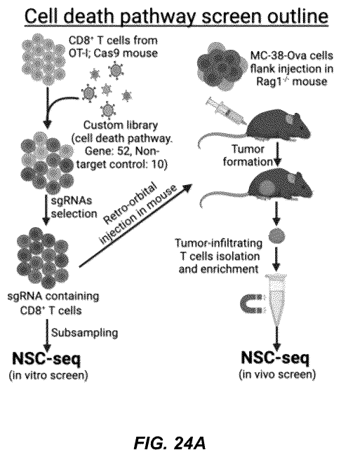

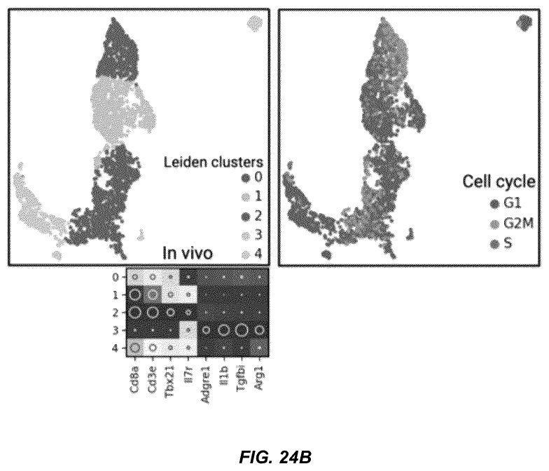

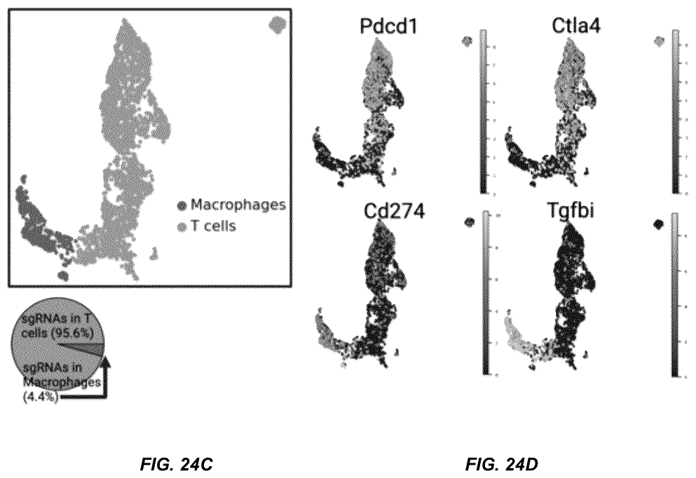

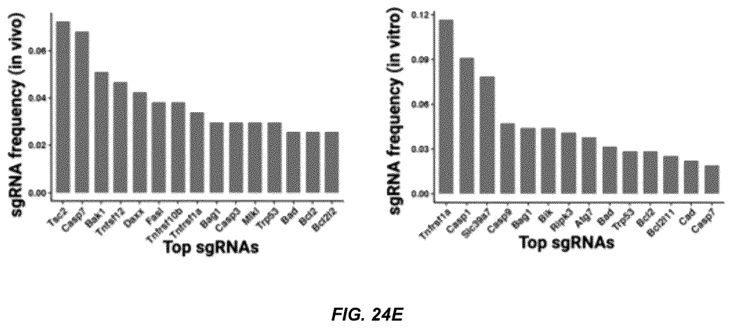

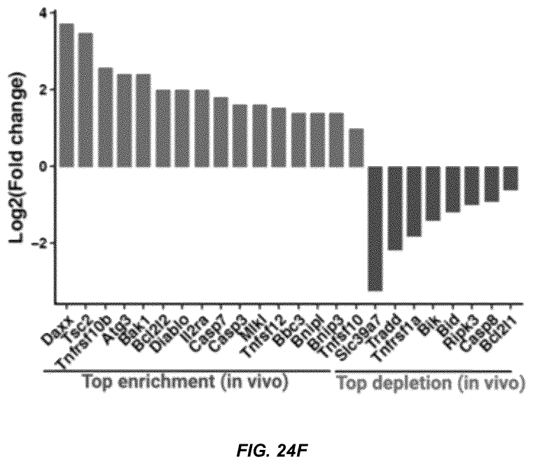

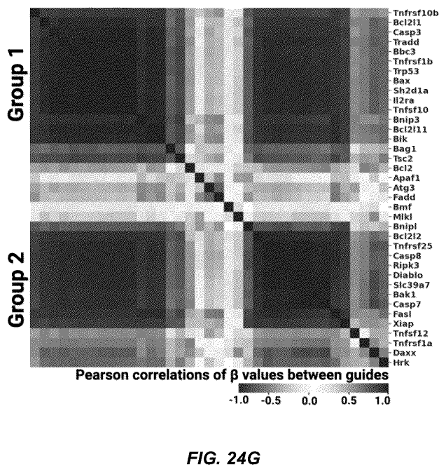

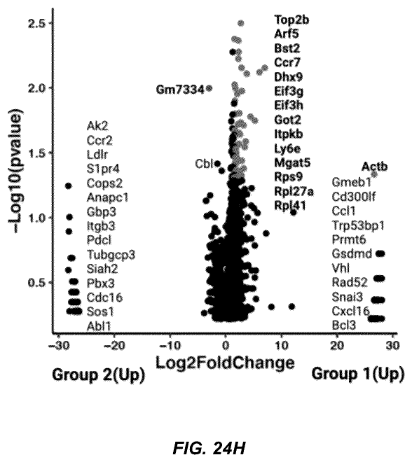

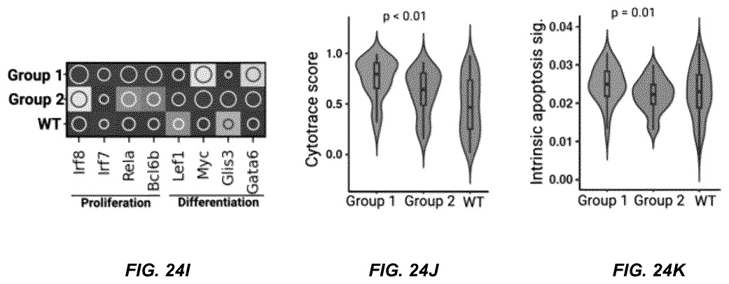

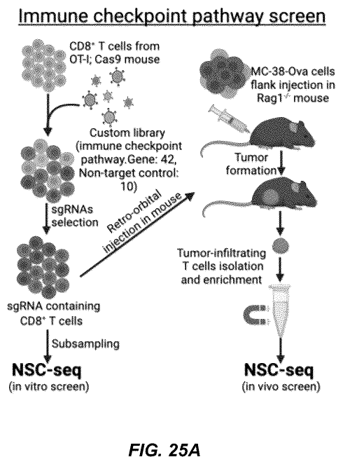

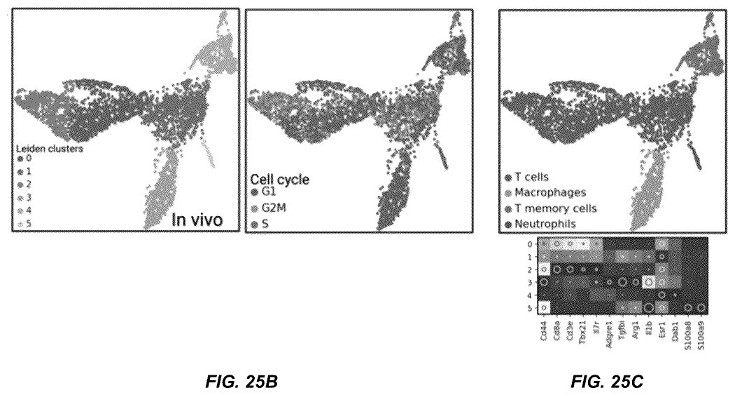

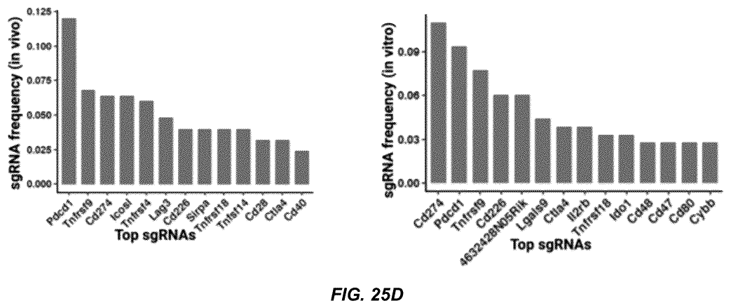

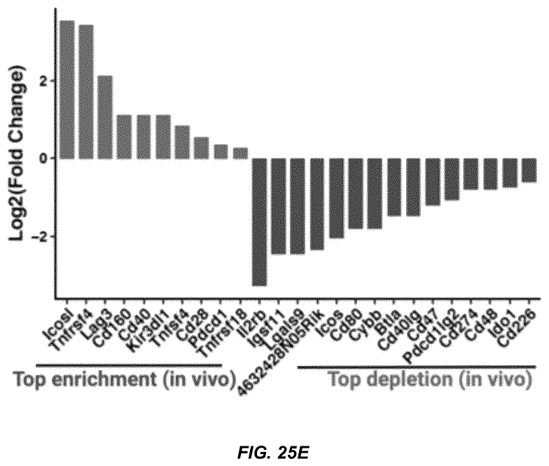

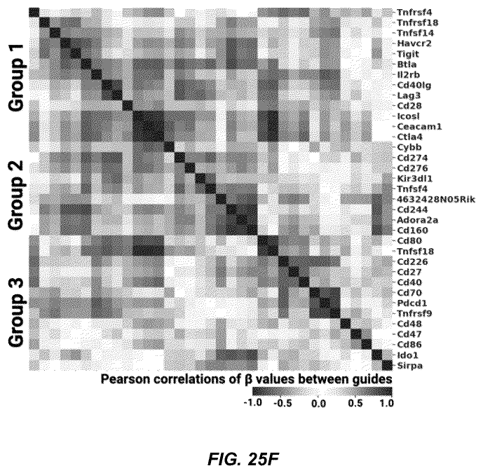

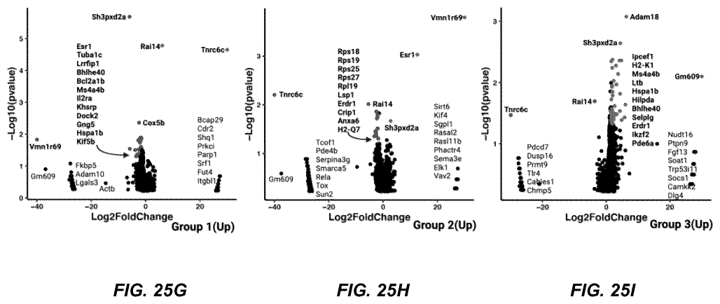

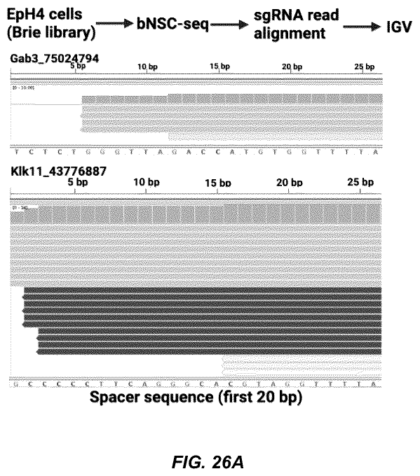

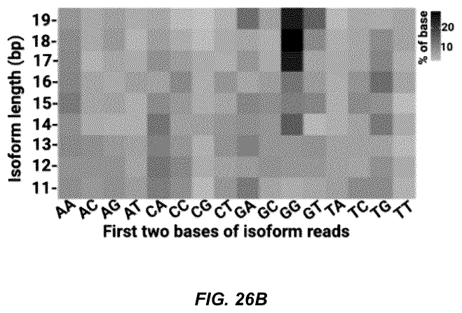

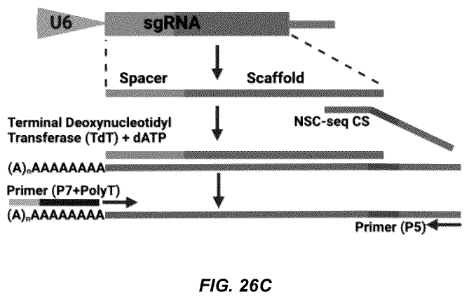

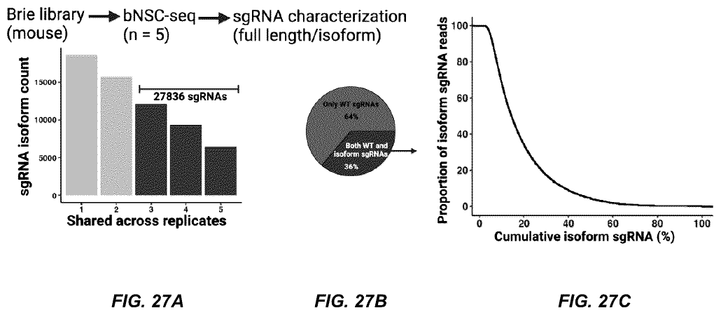

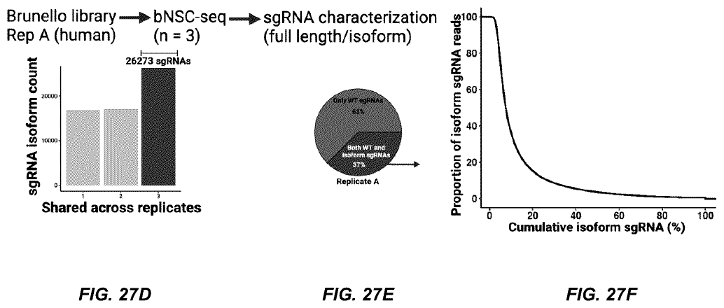

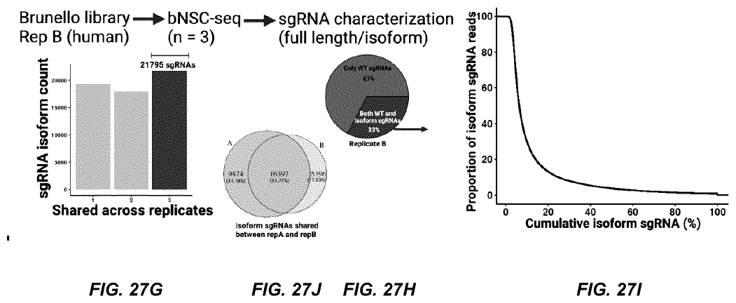

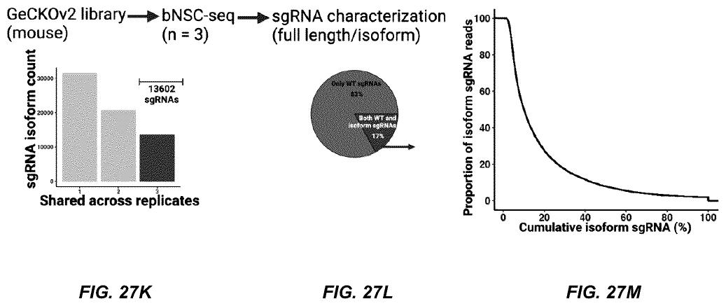

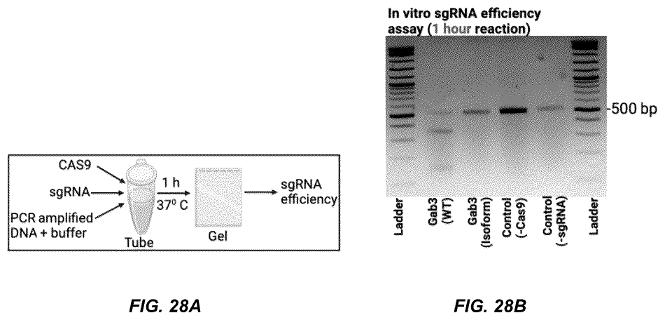

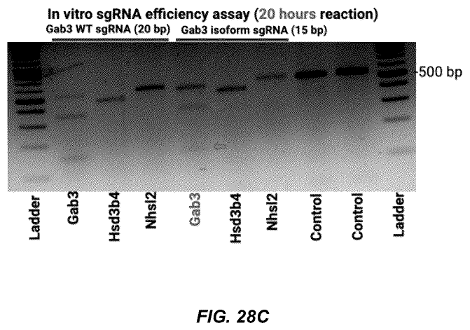

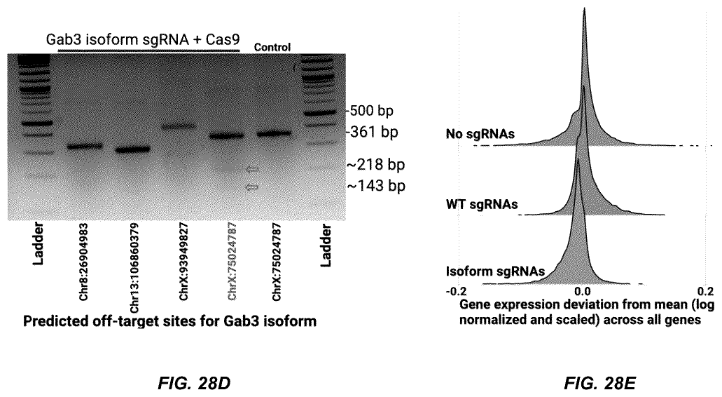

We then performed a gene essentiality screen using the mouse colorectal cell line MC38 with a custom sgRNA library to assess metabolic dependency of cancer cells (FIG. 23A) [Altman, B. J., et al. Nat Rev Cancer, 2016. 16(10):619-34]. After targeting components within the glutamate pathway, we found that the surviving cells were highly proliferative, residing mostly in G2M and S cell cycle phases. Despite similar proliferation kinetics, surviving cells displayed heterogeneity, with distinct regulon activities characterizing Leiden clusters 0 and 2, (FIG. 23B-23C). sgRNA enrichment analysis revealed five essential genes in the glutamate pathway (Got2, Ppat, Gfpt1, Gclc, and Ctps), as their knockout led to the elimination of targeted cells from the pool (FIG. 23D). Among the non-essential genes, we revealed two functional modules with distinct nucleotide metabolism gene enrichment, with glutamate receptor Grik4 being upregulated in module 2 (FIG. 23E). These results imply a compensatory mechanism for increasing glutamate uptake to confer a fitness advantage to cancer cells (FIG. 23F-23G) [Reinfeld, B. I., et al., Nature, 2021. 593(7858):282-288].

Finally, we performed two in vivo pooled single-cell CRISPR screens using mouse primary CD8+ cytotoxic T cells (CTLs). Perturbed CTLs were adoptively transferred into a MC38 tumor xenograft model to assess CTL infiltration and proliferation in the tumor microenvironment (TME) (FIG. 24A-24B) [Waldman, A. D., et al. Nat Rev Immunol, 2020. 20(11):651-668]. Isolation of immune cells within the TME showed a predominant CTL and tumor-associated macrophage (TAM) mixture, with CRISPR sgRNA only detected in CTLs, as expected (FIG. 24C). The TME exhibited features of immune exhaustion, with CTLs expressing Pdcd1 and Ctla4 [Buchbinder, E. I. and A. Desai, et al. Am J Clin Oncol, 2016. 39(1):98-106], and TAM expressing immunosuppressive genes such as Cd274 and Tgfbi (FIG. 24D) [Gordon, S. R., et al., Nature, 2017. 545(7655):495-499]. Targeting genes in the cell death pathway using this system confirmed previously reported antiproliferative mechanisms, such as Tsc2 as one of the top enriched genes that conferred a fitness advantage to CTLs in the TME (FIG. 24E) [Sugiura, A., et al., Immunity, 2022. 55(1):65-81.e9]. The zinc transporter Slc39a7 was found to be the most depleted gene, 147 supporting its essential role in lymphocyte development, as reported previously (FIG. 24F) [Anzilotti, C., et al., Nat Immunol, 2019. 20(3):350-361]. Overall, two distinct modules in cell death pathway were revealed to affect fitness in the TME, one modulating proliferation and the other modulating differentiation (FIG. 24G-24J). An intrinsic apoptotic signature was found in the proliferative module, implicating a vicious cycle of proliferation and apoptosis that leads to dysfunctional CTLs (FIG. 24K) [Horton, B. L., et al., Cancer Immunol Res, 2018. 6(1):14-24].

We performed a similar in vivo CTL screen using a custom immune checkpoint pathway library (FIG. 25A-25C). Our analysis revealed II2rb as one of the most depleted genes in the screen, as expected (FIG. 25D-25E) [Alderdice, M., et al., NAR Genom Bioinform, 2021. 3(2): Iqab016; Fernandez, I. Z., et al., J Exp Med, 2019. 216(6):1255-1267; Chinen, T., et al., Nat Immunol, 2016. 17(11):1322-1333]. Moreover, we found that perturbation of stimulatory signal receptors (Icos and Cd80) fostered CTL depletion, whereas perturbation of Icosl fostered CTL enrichment (FIG. 25E) [Raskov, H., et al., Br J Cancer, 2021. 124(2):359-367]. Ceacam 1 perturbation showed a similar transcriptome to Ctla4 perturbation, suggesting parallel functions for these genes in the immune checkpoint pathway (FIG. 25F) [Dankner, M., et al., Oncoimmunology, 2017. 6(7): e1328336]. Gene module analysis revealed genes associated with suppressing CTL activation in regulatory module 1 owing to immune suppressive genes (Itgbl1, Fut4, Prkci, and Parp1), whereas module 3 was characterized by T cell activation owing to T cell activation and differentiation-related genes (Ms4a4b, Socs1, Cd81, and Cd2001) (FIG. 25G-251) [Raskov, H., et al., Br J Cancer, 2021. 124(2):359-367; Dankner, M., et al., Oncoimmunology, 2017. 6(7): e1328336; Palmer, D. C. and N. P. Restifo. Trends Immunol, 2009. 30(12):592-602]. Overall, our results demonstrate that NSC-seq can be used to dissect CTL gene network regulation that modulates antitumor immunity [Zhou, P., et al., Nature, 2023].

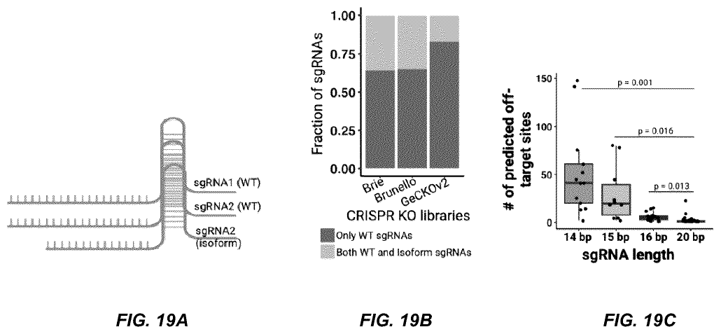

The foundational assumption in CRISPR screening is that the gRNAs expressed by individual cells are functional. Consequently, any observed enrichment or depletion of a gRNA during the screening process can be attributed to the effectiveness of a gRNA in perturbing the target gene. Since NSC-seq directly captures gRNAs rather than proxy barcodes embedded in DNA [Dixit, A., et al., Cell, 2016. 167(7):1853-1866.e17; Adamson, B., et al., Cell, 2016. 167(7):1867-1882.e21], we assessed the quality of expressed gRNAs in cells directly. Surprisingly, many instances of truncated spacer sequences, often missing bases from the 5′-end, were observed; we termed these alternative sequences sgRNA isoforms (FIGS. 19A, 28A). It has been reported that the U6 promoter displays a differential nucleotide preference for the transcription start site (TSS) [13, 14], which we also observed as base bias in sgRNA isoforms.

For instance, we found ‘GG’ dinucleotide as the dominant first two bases of isoforms from the Brie library (FIG. 26B). We then used an orthogonal approach to confirm isoform gRNA expression using terminal deoxynucleotide transferase (TdT) reactions (FIG. 26C). Here, we also observed truncated gRNA isoforms with a higher proportion of ‘GG’ dinucleotides as the first two bases (FIG. 26D-26E).

We then characterized the prevalence of this phenomenon across three whole genome KO screening libraries (FIG. 27). We found that a significant proportion (17-35%) of sgRNAs were expressed as both full length and truncated isoforms with variable degrees of isoform reads (FIGS. 19B, 27). To test whether sgRNA isoforms are functional, we assayed for their gene-editing efficiency and found compromised efficiency for sgRNA isoforms (15 bp length) compared to their WT counterparts (20 bp length) (FIG. 28A-28C) [Liu, X., et al., The Crop Journal, 2022. 10(2):577-581]. Furthermore, truncated isoforms may have reduced specificity due to length-dependent complementarity, causing off-target effects across the genome (FIG. 19C) [Ren, X., et al., Cell Rep, 2014. 9(3):1151-62; Fu, Y., et al., Nat Biotechnol, 2013. 31(9):822-6; Hsu, P. D., et al., Nat Biotechnol, 2013. 31(9):827-32]. Notably, we found that the Gab3-targeting sgRNA isoform edits one of the four predicted off-target sites (FIG. 28D). Finally, we assessed the effect of sgRNA isoforms on global transcriptional readouts and found a more significant effect on the transcriptome conferred by sgRNA isoforms (which could have broader off-targets) when compared with WT sgRNAs (FIG. 28E). Thus, our data reveal that truncated sgRNA isoforms exist in KO screening libraries which can potentially compromise the quality of CRISPR screens [Zhang, J. P., et al., Sci Rep, 2016. 6:28566; Josephs, E. A., et al., Nucleic Acids Res, 2015. 43(18):8924-41].

In summary, the NSC-199 seq platform provides a framework for targeted capture of non-polyadenylated gRNAs. We showed that conventional KO screen libraries can be used for pooled single-cell CRISPR screens via NSC-seq. As proof-of-principle, we provided confirmatory evidence in multiple in vitro and in vivo single-cell screens that revealed new insights into gene essentiality, gene function, and regulatory network modules in a variety of applied biomedical contexts. More importantly, direct gRNA readouts provided by NSC-seq can be used to assess the nucleic acid sequence of the sgRNA itself. Discordant/truncated sgRNAs compromise the efficacy and specificity of gene editing, which, on a genome scale, can detrimentally affect the quality of a screen. sgRNA isoforms can originate from multiple mechanisms, including inherent sgRNA sequence biases, viral insertion position biases in the genome, alternative TSS sequence biases, and degradation biases at the 5′-end of sgRNAs that lead to differential stability. Irrespective of the underlying cause, truncated sgRNA isoforms can lead to functional consequences in the downstream screening process. For genotype phenotype mapping of rare cell types, direct sgRNA readout could be a better proxy than DNA readout due to a higher copy number of sgRNAs per cell, which enhances detection. Our large scale sgRNA expression characterization of three widely used whole genome KO libraries provides reference datasets for truncated sgRNA profiles. Crosschecking across sgRNAs reference datasets might help in the design of better sgRNAs that minimize false positive discoveries.

Our multi-purpose platform is scalable and can be broadly applied beyond single-cell CRISPR screens. In a companion study, we applied this platform for the simultaneous lineage and temporal recording of mammalian development and precancer [Islam, M., et al., bioRxiv. 2023 Dec. 19:2023.12.18.572260]. Further development of NSC-seq can enable combinatorial genetic perturbations using KO plasmid libraries that can be multiplexed with barcoding for simultaneous in vivo genetic perturbation and lineage tracking [Santinha, A. J., et al., Nature, 2023; Michlits, G., et al., Nat Methods, 2017. 14(12):1191-1197]. Overall, we envision that the NSC-seq platform will expand the application of CRISPR approaches by facilitating genotype-phenotype mapping at scale with low cost and high flexibility.

### Methods and Materials

Native sgRNA Capture and Sequencing:

To capture the single guide RNA (sgRNA), a primer sequence termed capture sequence (CS) was designed to generate complementary DNAs (cDNAs). The CS is complementary to the 3′-end of the scaffold, enabling cDNA synthesis from self-mutating CRISPR gRNA (hgRNAs/stgRNAs) with complementary scaffold sequence, as reported in a companion study [Bock, C., et al., Nat Rev Methods Primers, 2022. 2(1)]. The spacer sequence at the 5′-end of sgRNAs exhibits variability depending on gene of interest, resulting in a lack of a constant sequence suitable for use as a primer to amplify cDNAs. To address this technical challenge, a unique reverse transcriptase activity known as template switching was used to facilitate the appending of a primer sequence to the 5′-end of the cDNAs. These primer pairs enabled cDNA amplification and subsequent Illumina sequencing library preparation (FIG. 18A).

sgRNA Amplification and Library Preparation:

High-quality total RNA was extracted from SW620 cells with Brunello libraries using QIAGEN RNA extraction kits (RNeasy Plus). Reverse transcription was carried out using Maxima H Minus Reverse Transcriptase (Catalog No: EP0751) and primers including CS primer with barcode and UMI, in addition to template switching oligo (TSO) primer. After primer digestion using Exonuclease I (NEB #M0568) and cleanup using AMPure (Catalog No: A63881), cDNAs were PCR amplified and indexed (Illumina sequencing adapters). Finally, the PCR-amplified product was gel-purified (˜350 bp) using QIAquick Gel Extraction kit. Sequencing libraries were quantified using NanoDrop. Duel-indexed libraries were pooled and loaded onto Illumina NovaSeq 6000 S4 flow cell using a PE150 kit.

### Single-Cell NSC-Seq Platform Optimization:

A single cell NSC-seq experiment was performed using the inDrops microfluidic platform [Shalem, O., et al., Science, 2014. 343(6166):84-87] as described in Star Protocols [Dixit, A., et al., Cell, 2016. 167(7):1853-1866.e17] and in a companion study [Bock, C., et al., Nat Rev Methods Primers, 2022. 2(1)] to enable the capture of sgRNA. In brief, custom hydrogel beads were designed using inDrop v2 chemistry [Shalem, O., et al., Science, 2014. 343(6166):84-87] that contain two types of capture sequence primers, PolyT (70%) for regular polyadenylated mRNA capture and NSC-seq CS (30%) for sgRNA capture in single-cell experiment. Note that 70:30 ratio yielded better sgRNA capture efficiency without compromising mRNA capture. The RT/Lysis buffer was customized to incorporate a 2.5 mM template-switch oligo (TSO) primer. Reverse transcription was performed at 50° C. for 1 hour. Sequencing libraries were prepared as described before [Adamson, B., et al., Cell, 2016. 167(7):1867-1882.e21], with modifications in the size selection step to yield a second cDNA pre-library enriched for barcoded sgRNAs between 250 and 350 bp (0.8×-1.2× double AMPure size selection). The sgRNA libraries were processed similarly to transcriptomic libraries, with the exception of fragmentation. Subsequently, the sgRNA libraries were PCR amplified using indexing primers (additional 8 cycles) and gel purified to obtain a ˜270 bp band using the QIAquick Gel Extraction kit. The dual-indexed libraries were combined and loaded onto an Illumina NovaSeq 6000 S4 flow cell using a PE150 kit, aiming for 100 million reads for transcriptome libraries and 50 million reads for sgRNA libraries [Datlinger, P., et al., Nat Methods, 2017. 14(3):297-301]. It's important to note that sgRNA libraries were not utilized for the single-cell RNA sequencing (scRNA-seq) count matrix.

sgRNA and mRNA Capture Efficiency:

sgRNA capture efficiency was estimated in both bulk and single-cell levels. Cas9 expressing SW620 cells were transduced by Brunello (Addgene: 73178) lentivirus pooled library using previously reported approach [Jaitin, D. A., et al., Cell, 2016. 167(7):1883-1896.e15]. After drug selection of the transduced cells, each plate was divided into two fractions, one plate for bulk DNA extraction and other plate for bulk total RNA extraction using kits (QIAGEN). The sgRNA sequences were PCR amplified from bulk genomic DNA using recommended adaptor primers, followed by amplification using Illumina indexing primers. Purified PCR produced were sequenced using NovaSeq6000 and analyzed sgRNA recovery using custom scripts. Similarly, bulk total RNAs were used (˜100 μg) for sgRNA capture using NSC-seq CS, followed by PCR amplification using indexing primers and sequenced by NovaSeq6000. Data were analyzed using custom scripts. Percent of total sgRNAs in Brunello library was calculated and found to be comparable sgRNA capture efficiency between bulk NSC-seq and DNA sequencing approach, supporting robustness of CS for sgRNA capture and recovery (FIGS. 18B and 20B). Single-cell level sgRNA capture efficiency has been reported in a companion study [Bock, C., et al., Nat Rev Methods Primers, 2022. 2(1)]. Briefly, mouse mammary epithelial cells (EpH4) were transduced with Brie library (Addgene: 73633) using a previously reported approach [Jaitin, D. A., et al., Cell, 2016. 167(7):1883-1896.e15]. Three encapsulated fractions of these cells were processed, and libraries were prepared using different size selection approaches. Double size selection (0.8×-1.2×), followed by two independent library preparation approaches enabled more than 95% sgRNA recovery rate. Transcriptome from these NSC-seq libraries were compared with inDrop libraries (wild-type EpH4 cells) using previously described [Replogle, J. M., et al., Nat Biotechnol, 2020. 38(8):954-961] approach and found to have similar transcriptome capture efficiency between NSC-seq beads and inDrop beads in duplicate scRNA-seq libraries. Moreover, the NSC-seq approach was applied to multiple embryonic time points, fetal and adult intestinal epithelium, intestinal tumor and found a high-quality transcriptome (companion study). Thus, NSC-seq platform ensures a robust sgRNA capture efficiency without compromising transcriptome quality.

### Transcriptome Sequencing, Alignment, and Quality Control:

Sequence alignment was conducted using a previously reported approach [8]. Briefly, the DropEst pipeline [Sanson, K. R., et al., Nat Commun, 2018. 9(1):5416] utilized the STAR aligner [Hart, T., et al., G3 (Bethesda), 2017. 7(8):2719-2727] with the Ensembl reference genome to map reads to the genome, quantify transcript abundance and generate a cells-by-genes counts matrix that was processed as a scanpy AnnData object [Hill, A. J., et al., Nat Methods, 2018. 15(4):271-274]. High-quality, cell-containing droplets were identified through finding the inflection point of the cumulative sum curve, and droplets with low information content were removed as reported before [Datlinger, P., et al., Nat Methods, 2017. 14(3):297-301]. The full protocol for running this QC pipeline is described by Chen et al. [Dixit, A., et al., Cell, 2016. 167(7):1853-1866.e17]. Genes that were detected in fewer than 5 cells and potential doublets detected by scrublet [Jinek, M., et al., Science, 2012. 337(6096):816-21] were removed. Cells with high mitochondrial and ribosomal count percentages were also removed. The filtered count matrix was normalized to median library size. Normalized counts were log transformed for variance stabilization. For each dataset, 4000 Highly Variable Genes (HVGs) were identified for downstream analysis using seurat v3 [Gao, Z., et al., Transcription, 2017. 8(5):275-287] and the number of components was selected by the elbow method [Ma, H., et al., Mol Ther Nucleic Acids, 2014. 3(5): e161]. Batch correction was performed using Harmony [Perli, S. D., et al. Science, 2016. 353(6304)].

### Low-Dimensional Embedding and Unsupervised Clustering:

A k-Nearest Neighbors (kNN) graph was constructed with 25 neighbors using Euclidean distance in the principal components space as the inputs. A two-dimensional Uniform Manifold Approximation and Projection (UMAP) embedding was generated for all NSCseq datasets. Single-cell subclusters were labeled in UMAP using the Leiden algorithm, as part of the Scanpy toolkit. Some cell types were assigned using scMRMA [Kalhor, R., et al. Nat Methods, 2017. 14(2):195-200] and were further verified using cell-type-specific marker genes.

### Custom Knockout Screen Library Construction:

Custom sgRNA libraries (glutamate, cell death, and immune checkpoint pathways) were constructed using previously reported approach [Kalhor, R., et al., Science, 2018. 361(6405)]. Briefly, spacer sequences were curated from the Brie Library (Addgene: 73632) with four sgRNAs per gene and ten nontargeting controls per libraries. Oligo pools were purchased from Twist Bioscience and cloned into the retroviral expression vector pMx-U6-gRNA using Gibson Assembly Master Mix. Plasmid DNA was transfected into Plat-E retroviral packaging cell line, media changed after 24 h, and viral supernatant was collected after additional 48 h.

### In Vitro Single-Cell CRISPR Screens:

Three in vitro screens were performed using three different cell lines (EpH4, SW620, and MC38). Cas9 expressing EpH4 cells were transduced using Brie lentivirus pooled library (Addgene: 73632), followed by puromycin selection. Lentivirus were prepared using previously reported approaches [Jaitin, D. A., et al., Cell, 2016. 167(7):1883-1896.e15; Islam, M., et al., bioRxiv. 2023 Dec. 19:2023.12.18.572260]. Cells were encapsulated using NSC-seq platform, followed by library preparation and sequencing. Similarly, Cas9 expressing SW620 cells were transduced using Brunello (Addgene: 73178) lentivirus pooled library using standard protocol. After positive selection, SW620 cells were treated using trametinib (MEK1/2 inhibitor) with same concentration in control plate (SW620-Cas9 only). Under continuous drug selection and culture condition, trametinib resistant cells were appeared in lentivirus infected plate, while cells in the control plate cells were dead. Resistant cells were encapsulated using NSC-seq platform, followed by library preparation and sequencing. Similarly, the glutamate KO viral supernatant was transduced into MC38 cells in culture plates. Infected cells were enriched using FACS (BFP+) and encapsulated after 1 week in culture.

### In Vivo Single-Cell CRISPR Screens:

Splenocytes were isolated from double transgenic mice (OT-I; Cas9) and activated with SIINFEKL peptide and IL-2. CD8⁺ T cells were isolated following 48 hours of activation and transduced by retroviral supernatant using a previously reported approach [Kalhor, R., et al., Science, 2018. 361(6405)]. A fraction of these in vitro cells was encapsulated using NSC-seq platform before injection, followed by library preparation and sequencing. Meanwhile MC-38-ova cells were injected into flank of Rag1^{-/-} mice to grow xenograft. After observing visible tumors in mice flank, 10 million live CD8+ T cells with custom sgRNA library were transferred by retro-orbital injection into Rag1^{-/-} mice. After 1 week of injection, the tumors were collected, and the T cells were isolated and enriched using Cd8 microbeads. These cells were encapsulated using NSC-seq platform, followed by library preparation and sequencing.

sgRNA Identity Mapping:

An in-house pipeline using Python and R scripts was employed to analyze sgRNA sequencing data. Briefly, sgRNA sequences were extracted from paired-end reads using 12 bp constant sequence from sgRNA scaffold region and the reads from R1 and R2 were mapped using read headers. Single-cell barcode IDs were extracted from R1, whereas sgRNA spacer sequences were extracted from R2. R2 reads were trimmed in between TSO sequence and scaffold sequence to get spacer sequence (20 bp). UMI sequences (6-bases) were extracted from R2 to identify PCR duplication. Spacer sequences with unique cell ID were assigned to specific sgRNA using known spacer sequence of the corresponding sgRNA libraries (see GitHub repository for more details).

### Single-Cell CRISPR Screen Data Analysis:

Single-cell gene expression matrix was generated using previously reported approach. Single cell sgRNA matrix was generated using custom scripts (NSC-seq GitHub page). Cells with >1 sgRNAs and truncated sgRNAs (isoform) were filtered out for downstream analysis. Data were analyzed using previously reported linear model from Perturb-seq paper [Solimini, N. L., et al., Proc Natl Acad Sci USA, 2013. 110(5): E407-14]. Briefly, cell state and cell cycle status were added as covariates in designed sgRNA matrix. Regulatory coefficients (beta) were generated for each sgRNAs using gene expression matrix and guide-gene mapping matrix as inputs for linear model. Finally, a pairwise coefficient matrix was visualized to identify functionally similar sgRNA clusters.

Whole-Genome Scale Truncated sgRNA Characterization:

Three whole genome CRISPR KO screening libraries (Brie (Addgene: 73633), Brunello (Addgene: 73178), GeCKOv2 (Addgene: 1000000052)) were used for characterization of truncated sgRNAs (FIG. 27). Lentivirus were prepared using previously reported approaches [Jaitin, D. A., et al., Cell, 2016. 167(7):1883-1896.e15; Islam, M., et al., bioRxiv. 2023 Dec. 19:2023.12.18.572260]. Brie and GeCKOv2 libraries were transduced into EpH4 cells, whereas Brunello library was transduced into SW620 cells. After positive initial selection of plasmid transduced cells, bulk total RNAs were extracted from the mentioned cell lines (QIAGEN). The total RNAs were used for bulk NSC-seq using similar approach as reported before. An in-house pipeline using R scripts was employed to analyze and annotate the truncated sgRNAs across three popular libraries. Briefly, bulk sgRNA sequencing data were analyzed from each technical and/or biological replicate separately. sgRNA reads were extracted using a 10 bp constant sequence and trimmed up to the spacer sequence. Duplicate spacer reads were merged after extracting UMI from each read and the remaining sequences were considered for spacer annotation. A spacer was assigned as a wild type (WT), when the spacer sequence (20 bp) was exactly matched with reference spacer sequence (20 bp) of specific library. The remaining unmatched spacer reads were considered for truncated sgRNA annotation. The unmatched spacer reads were trimmed into the last 19 bp and matched with similar 19 bp of reference spacer sequence to assigned truncated 19 bp sgRNAs. Similarly, the truncated sgRNAs were assigned until 11 bp of spacer sequence with custom R script. Note that, this process initially would assign PCR and/or sequencing artifact as truncated (isoform) sgRNAs category. The possibility of having PCR and/or sequencing errors in the same base position, same spacer read, and across multiple independent replicates would be insignificant. Thus, the assigned truncated sgRNAs were further filtered out if they were not shared in more than two independent technical replicates (FIG. 27). Moreover, sgRNA reads from Brie library were aligned to custom reference genome (spacer sequences) using BWA [Kataoka, T., et al., J Biol Chem, 2001. 276(22):19548-54]. Selective sgRNAs were visualized using IGV (FIG. 28A) [Hart, T., et al., Cell, 2015. 163(6):1515-26].

Truncated sgRNAs Validation:

Terminal Deoxynucleotidyl Transferase (TdT) reaction (Catalog #EP0161) was used as an orthogonal approach to validate truncated sgRNA expression under U6 promoter. Briefly, total RNA with self-mutating gRNA from HEK293 cells was used for the cDNA synthesis reaction using CS primer and Maxima H Minus Reverse Transcriptase (Catalog #EP0752). Instead of using templet switch oligo to add PCR primer handle at the 3′-end of the cDNA, TdT enzymes with dATP were used to add polyA tail at the 3′-end of the cDNA (FIG. 28C). Recommended manufacturers protocol (Thermo Fisher Scientific) was used for the tailing of DNA 3′-termini reaction at 37° C. for 30 min and inactivated the reaction by heating at 700 C for 10 min. cDNA amplification was carried out with custom P5 primer and polyT (n=15) conjugated P7 primer, followed by indexing and sequencing using NovaSeq 6000 and PE150 kit. Data were analyzed using custom pipeline and representative isoform reads were shown in FIG. 28D.

Gene Editing Efficacy and Precision of Truncated sgRNA:

Custom sgRNAs were designed from IDT for full form (20 bp) and isoform (15 bp) crRNA (Brie library Gab3 spacer, position of base after cut (1-base): 75024794). In addition, tracerRNA was also designed from IDT. Lyophilized Cas9 protein was purchased from PNA Bio, Inc (Lot #PCN28910). Targeted mouse DNA loci (WT) were PCR amplified and purified for the reaction using primer sequences (Supplemental table 1). In vitro DNA digestion reaction was performed according to previous protocol [Wu, P. K. and J. I. Park. Semin Oncol, 2015. 42(6): 849-62] with modification. Reaction was carried out for 1 h or 20 h at 37° C. in PCR machine. Control was either no CAS9 protein or no sgRNA in the reaction mixture. DNA cleavage activity was visualized by running the reaction in agarose gel (2%). Gab3 isoform off-target sites were predicted using online tool [Alarcón, T. and H. J. Jensen. J R Soc Interface, 2011. 8(54):99-106]. Four genomic locus was PCR amplified for precision editing. Cas9 digestion reactions were carried out using reported approach for 20 h. One of the four locus was found to be cleaved by Gab3 isoform sgRNA (FIG. 28).

### Gene Expression Deviation Calculation:

Three selective single-cell groups (No sgRNAs, WT sgRNAs, and Isoform sgRNAs) were curated from fitness screen (EpH4 cells) with similar number of cells per group (FIG. 21). Cells with a single detected sgRNA (WT/isoform) were remained in the matrix. Combined gene expression matrix was normalized to median library size, followed by log transformed and scaling. The global mean expression per gene (pseudo-bulk) was calculated from the transformed matrix. The gene expression deviation was calculated by subtracting each of the group mean expression per gene from the global mean expression of that gene. The deviation values for all genes were plotted as density plot in FIG. 28D.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A gRNA capture primer comprising from 3′ to 5′ a capture polynucleotide complementary to the 3′-end of a gRNA scaffold having the nucleic acid sequence GACTCGGTGCCACTTTTTCAAG (SEQ ID NO:1), a cellular barcode, a unique molecular identifier (UMI), and optionally a T7 promoter sequence.

2. A native gRNA capture and sequencing (NSC-seq) method for single-cell RNA profiling of pooled gRNA comprising:
(a) reverse transcribing the pooled gRNA with a native gRNA capture and sequencing (NSC-seq) primer comprising from 3′ to 5′ a capture polynucleotide complementary to the 3′-end of the gRNA scaffold having the nucleic acid sequence GACTCGGTGCCACTTTTTCAAG (SEQ ID NO: 1), a cellular barcode, and a unique molecular identifier (UMI) to produce a cDNA;
(b) adding an additional sequence to the 3′ end of the cDNA during reverse transcription using a template switch oligonucleotide for library amplification;
(c) amplifying the cDNA by polymerase chain reaction (PCR); and
(d) sequencing the cDNA.

3. A method for single-cell screening of pooled gRNA perturbations comprising:
(a) introducing one or more gRNA perturbation constructs encoding for one or more sequence specific perturbations to a plurality of cells in a population of cells, wherein each cell in the plurality of the cells receives at least 1 perturbation and wherein each gRNA perturbation construct encodes for an RNA sequence comprising a sequence identifying the perturbation;
(b) detecting endogenous mRNAs for each single cell in the plurality of cells using single-cell RNA-seq;
(c) detecting gRNA perturbations for each single cell in the plurality of cells using the NSC-seq method of claim 2; and
(d) comparing gRNA perturbations to mRNA for each single cell.

4. The method of claim 3, wherein the gRNA is a self-mutating homing guide RNA (hgRNA).

5. The method of claim 4, wherein the method further involves temporal tracking mutations in the hgRNA over cell divisions in a single organism.

6. The method of claim 4, wherein the method further involves lineage tracking mutations in the hgRNA to examine the relationships between organs and tissue layers in an organism.

7. The method of claim 3, wherein the population of cells comprises ex vivo or in vitro cells.

8. The method of claim 3, wherein each cell is in a microfluidic system; and/or wherein each cell is in a droplet.


SEQUENCE LISTING (19 sequences)

SEQ ID NO: 1 - RNA, 22 nt, synthetic construct
  source 1..22: mol_type = other RNA
gactcggtgccactttttcaag

SEQ ID NO: 2 - RNA, 38 nt, synthetic construct
  source 1..38: mol_type = other RNA
atcaacttgaaaaagtggcaccgagtcggtgctttttt

SEQ ID NO: 3 - RNA, 21 nt, synthetic construct
  source 1..21: mol_type = other RNA
ggtgtaacttaacactgagtg

SEQ ID NO: 4 - RNA, 21 nt, synthetic construct
  source 1..21: mol_type = other RNA
ggtttacgttgcatcggtgtg

SEQ ID NO: 5 - RNA, 18 nt, synthetic construct
  source 1..18: mol_type = other RNA
ggtcaggtcacgtctgtg

SEQ ID NO: 6 - RNA, 21 nt, synthetic construct
  source 1..21: mol_type = other RNA
ggttcccgtccagtaagcgtg

SEQ ID NO: 7 - RNA, 22 nt, synthetic construct
  source 1..22: mol_type = other RNA
ggttatatttgtaagtgtagtg

SEQ ID NO: 8 - RNA, 22 nt, synthetic construct
  source 1..22: mol_type = other RNA
ggtgtaacttaacactgatgtg

SEQ ID NO: 9 - RNA, 21 nt, synthetic construct
  source 1..21: mol_type = other RNA
ggtttacgttgcatcggtgtg

SEQ ID NO: 10 - RNA, 18 nt, synthetic construct
  source 1..18: mol_type = other RNA
ggtcaggtcacgtctgtg

SEQ ID NO: 11 - RNA, 19 nt, synthetic construct
  source 1..19: mol_type = other RNA
ggttcccgtccaaagcgtg

SEQ ID NO: 12 - RNA, 22 nt, synthetic construct
  source 1..22: mol_type = other RNA
ggttatatttgtaagtgtagtg

SEQ ID NO: 13 - RNA, 18 nt, synthetic construct
  source 1..18: mol_type = other RNA
ggtgtaacttctgatgtg

SEQ ID NO: 14 - RNA, 19 nt, synthetic construct
  source 1..19: mol_type = other RNA
ggtttacgttgcatcggtg

SEQ ID NO: 15 - RNA, 20 nt, synthetic construct
  source 1..20: mol_type = other RNA
ggtcaggtcttacgtctgtg

SEQ ID NO: 16 - RNA, 19 nt, synthetic construct
  source 1..19: mol_type = other RNA
ggttcccgtccaaagcgtg

SEQ ID NO: 17 - RNA, 20 nt, synthetic construct
  source 1..20: mol_type = other RNA
ggttatatttgtaagtagtg

SEQ ID NO: 18 - RNA, 26 nt, synthetic construct
  source 1..26: mol_type = other RNA
tctctgggttagaccatgtggtttta

SEQ ID NO: 19 - RNA, 26 nt, synthetic construct
  source 1..26: mol_type = other RNA
gcccccttcagggcacgtaggtttta